(19) United States
(12) Patent Application Publication
(10) Pub. No.: US 2018/0271891 A1
(43) Pub. Date: Sep. 27, 2018

(54) SELECTIVE TREATMENT OF PRMT5 DEPENDENT CANCER

(71) Applicants: THE BROAD INSTITUTE INC., Cambridge, MA (US); DANA-FARBER CANCER INSTITUTE, INC., Boston, MA (US)

(72) Inventors: Levi A. Garraway, Newton, MA (US); Grigoriy Kryukov, Newton, MA (US); Jason Ruth, Boston, MA (US); Frederick Wilson, Boston, MA (US)

(21) Appl. No.: 15/557,411

(22) PCT Filed: Mar. 10, 2016
(86) PCT No.: PCT/US2016/021697

§ 371 Date: Sep. 11, 2017

Related U.S. Application Data
(60) Provisional application No. 62/131,825, filed on Mar. 11, 2015.
(60) Provisional application No. 62/252,277, filed on Nov. 6, 2015.

(51) Int. Cl. A61K 31/7076; A61K 31/4439; A61K 47/54; A61K 47/55; A61P 35/00; A61K 45/06; C12Q 1/48

(52) U.S. Cl. CPC A61K 31/7076; A61K 31/4439; A61K 47/549; A61K 47/545; C12Y 204/02028; A61P 35/00; A61K 45/06; C12Q 1/48; A61K 47/55

(57) ABSTRACT

The present invention generally relates to therapeutic inhibition of protein arginine methyltransferase 5 (PRMT5). In particular, cell lines having MTAP loss and increased intracellular MTA concentrations show selective dependence on PRMT5. Thus, the invention also relates to methods of identifying and treating PRMT5-related diseases in subjects or tissues which have an MTAP deficiency, alone or in combination, with a second agent that reduces MTAP activity and/or increases intracellular MTA levels, and/or provides an MTA analogs to the cell or tissue. The invention also relates to the Clustered Regularly Interspaced Short Palindromic Repeats (CRISPR)-Cas System and components thereof. More specifically, the present invention relates to the delivery, use and therapeutic applications of the CRISPR-Cas systems and compositions in tumor cells ex vivo and/or in vivo. For example using methods disclosed herein, cells can be sensitized to PRMT5 inhibition.

### RELATED APPLICATIONS AND INCORPORATION BY REFERENCE

This application claims priority to and benefit of US provisional patent application Ser. No. 62/131,825, filed Mar. 11, 2015 and U.S. provisional patent application Ser. No. 62/252,277, filed Nov. 6, 2015.

Reference is also made to G. V. Kryukov et al., MTAP deletion confers enhanced dependency on the PRMT5 arginine methyltransferase in cancer cells. Science DOI: 10.1126/science.aad5214, available online Feb. 11, 2016.

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under Grant Nos. CA197737 and CA163222 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 19, 2016, is named 46783.99.2109_SL.txt and is 10 bytes in size.

### FIELD OF THE INVENTION

The present invention generally relates to therapeutic inhibition of protein arginine methyltransferase 5 (PRMT5). In particular, cell lines having MTAP loss and increased intracellular MTA concentrations show selective dependence on PRMT5. Thus, the invention also relates to methods of identifying and treating PRMT5-related diseases in subjects or tissues which have an MTAP deficiency, using PRMT5 inhibitors alone or in combination with a second agent that reduces MTAP activity and/or increases intracellular MTA levels, and/or provides an MTA analog to the cell or tissue.

The invention also relates to the Clustered Regularly Interspaced Short Palindromic Repeats (CRISPR)-Cas System and components thereof. More specifically, the present invention relates to the delivery, use and therapeutic applications of the CRISPR-Cas systems and compositions in tumor cells ex vivo and/or in vivo. For example using methods disclosed herein, cells can be sensitized to PRMT5 inhibition.

### BACKGROUND OF THE INVENTION

The identification of genetic alterations that predict response to specific therapies can facilitate therapeutic design by identifying patients likely to receive maximal benefit from a drug. MTAP deletion has been postulated as a possible basis for therapeutic exploitation due to its involvement in the adenine salvage pathway. PRMT5 has recently emerged as a possible target in some cancers, but genetic alterations that determine or predict sensitivity to PRMT5 inhibition have not been identified.

Recent advances in genome sequencing techniques and analysis methods have significantly accelerated the ability to catalog and map genetic factors associated with a diverse range of biological functions and diseases. Further, precise genome targeting technologies are being developed to enable systematic engineering of genetic variations to allow selective perturbation of individual genetic elements, as well as to advance synthetic biology, biotechnological, and medical applications.

CRISPR-Cas9 is a powerful technology for genome editing and is being widely adopted due to its efficiency and versatility. While Cas9-mediated genome editing applications are compelling, applying them in vivo and ex vivo is challenging, since commonly used delivery systems are inefficient and limit accessible cell types. Moreover, certain cell types, e.g., primary immune cells present particular challenges and are often not accessible for genetic manipulation due to delivery challenges, short viability terms in culture, or both.

A major challenge facing the continued study of genetics is distinguishing which mutations are drivers from those that are not (“passengers”) (Garraway and Lander, 2013; Lawrence et al. 2013). The difficulty of elucidating these distinctions in animal models lies in precisely generating such mutation(s) and measuring the influence of specific mutations throughout the subject's condition. These challenges apply to other areas of genetic and tissue-specific biological studies as well.

The vast majority of genetic variants associated with human disease localize to noncoding regions of the genome, which bolsters the emerging view that perturbation of genetic regulatory elements disrupts interconnected transcriptional circuits and drives pathology. Of particular importance is the striking enrichment of disease single nucleotide polymorphisms (SNPs) found within super-enhancers, which control genes required for lineage commitment. Recent evidence clearly demonstrates the central role of super-enhancers in a wide range of disease processes including autoimmune responses.

Citation or identification of any document in this application is not an admission that such document is available as prior art to the present invention.

### SUMMARY OF THE INVENTION

There exists a need for methods for identifying genetic alterations that predict responses to specific therapies as well as methods of enhancing such therapies. In one aspect, the instant invention identifies MTAP loss as predicting and contributing to sensitivity of PRMT5 inhibition. As MTAP is ubiquitously expressed in healthy normal tissues and MTAP loss predicts sensitivity to PRMT5 inhibition across multiple cancer lineages, PRMT5 inhibition in vivo can preferentially ablate MTAP—tumor cells while sparing MTAP-expressing cells in normal tissues. The invention also provides diagnostic methods for identifying MTAP-deficient cells and tissues, which can be selectively targeted by PRMT5 inhibition. Furthermore, the invention provides methods for inhibiting, or inactivation of, MTAP in order to facilitate targeting with a PRMT5 inhibitor.

In one aspect, the invention provides a method of treating a neoplasm, such as a cancer or tumor, in a subject in which 5′-deoxy-5′-methylthioadenosine (MTA) levels are elevated, for example due to reduction or loss of methylthioadenosine phosphorylase (MTAP) activity, which comprises administering to the subject an effective amount of an inhibitor of protein arginine methyltransferase 5 (PRMT5). In preferred embodiments, MTA levels are elevated in the cancer or tumor cells and not in healthy cells of the subject. In certain such embodiments, the cancer or tumor cells lack MTAP. In other such embodiments, MTAP expression in the cancer cells is reduced or inhibited. In another aspect, the invention provides a method of treating a neoplasm in a subject, which comprises administering an effective amount of an inhibitor of PRMT5 and an effective amount of an agent that elevates MTA levels. In some such embodiments, the agent that elevates MTA levels is an inhibitor of MTAP. In other such embodiments, the level of MTA is raised or supplemented by providing MTA to the neoplasm.

In another aspect, the invention provides a method of treating a neoplasm, which comprises administering an effective amount of an inhibitor of PRMT5 and an effective amount an MTA analog, or derivative. In one such embodiment, the MTA analog or derivative is an inhibitor of PRMT5, and is not a substrate of MTAP. In another such embodiment, the MTA analog is both an inhibitor of PRMT5 and an inhibitor of MTAP.

In certain embodiments, an effective amount of a PRMT5 inhibitor is coadministered with one or more of MAT, an MTA analog or derivative, and an MTAP inhibitor.

In another aspect, the invention provides a method of treating a neoplasm in a subject, which comprises identifying the neoplasm as having cells that express a reduced amount of MTAP or no MTAP, and treating the subject with a PRMT5 inhibitor. In certain such embodiments, the cells of the neoplasm are homozygous for an MTAP deletion. In other such embodiments, the cells of the neoplasm are heterozygous for an MTAP deletion.

In another aspect, the invention provides a method of treating or preventing a PRMT5-mediated disorder in a subject, which comprises administering to subject an effective amount of 5′-deoxy-5′-methylthioadenosine (MTA) or MTA analog or an MTAP inhibitor. In certain such embodiments, an effective amount of an inhibitor of PRMT5 is also administered. Not being bound by a theory, MTAP cleaves MTA, MTA inhibits PRMT5 and MTAP is frequently knocked out in cancer. Therefore, if MTAP is knocked out or downregulated, there is an increase in MTA and the cancer cells are more sensitive to a PRMT5 inhibitor than healthy cells. Moreover, if MTAP is knocked out, then a cancer may be treated with MTA, whereby PRMT5 is selectively inhibited in cancer cells because MTA can not be cleaved in the cancer cells, but can be cleaved in healthy cells.

In certain aspects, a PRMT5-mediated disorder is a proliferative disorder, a metabolic disorder, including but not limited to diabetes or obesity, or a blood disorder, including but not limited to hemoglobinopathy, sickle cell anemia or β thalassemia.

In another aspect, the invention provides a method of identifying a suitable therapy for treatment of a neoplastic disease in a subject, which comprises measuring the level of methylthioadenosine phosphorylase (MTAP) activity in the tumor, and if the level of MTAP activity is reduced compared to normal cells, administering a protein arginine methyltransferase 5 (PRMT5) inhibitor. In yet another aspect, the invention provides a method of identifying a suitable therapy for treatment of a neoplastic disease in a subject, which comprises measuring the level of MTAP activity in the tumor, and if the level of MTAP activity is reduced compared to normal cells, administering a protein arginine methyltransferase 5 (PRMT5) inhibitor and MTA to the subject, wherein the PRMT5 inhibitor and MTA inhibitor are in amounts effective to inhibit proliferation of cells of the tumor.

Aspects of the invention provide methods for using the CRISPR-Cas system. In certain non-limiting embodiments, a plurality of sgRNAs is delivered (e.g., via various means including a vector (e.g., lentiviral vector), a particle (e.g., nanoparticle) into eukaryotic cells. Aspects of the present invention involve sequence targeting, such as genome perturbation or induction of multiple mutations using the Clustered Regularly Interspaced Short Palindromic Repeats (CRISPR) system or components thereof. The invention provides systematic reverse engineering of causal genetic variations, including through selective perturbation of individual, and moreover, multiple genetic elements. For instance, in non-human eukaryote, e.g., animal, such as fish, e.g., zebra fish, mammal, e.g., primate, e.g., ape, chimpanzee, macaque, rodent, e.g., mouse, rabbit, rat, canine or dog, livestock (cow/bovine, sheep/ovine, goat or pig), fowl or poultry, e.g., chicken, insect, arthropod or plant, e.g., dicot (e.g., nightshade such as tobacco, tuber such as potato) or monocot (e.g., corn) models that constitutively or through induction or through administration or delivery, have cells that contain Cas9. The invention provides tools for studying genetic interaction between multiple individual genetic elements by allowing selective perturbation of e.g., one or more immune system-associated or correlated gene(s)/genetic element(s). In an aspect, the invention provides methods for using one or more elements/components of a CRISPR-Cas system via a vector and/or particle and/or nanoparticle delivery formulation or system as a means to modify a target polynucleotide. In preferred embodiments, the delivery is via a viral vector (e.g., AAV, adenovirus, lentivirus). The CRISPR complex of the invention provides an effective means for modifying a target polynucleotide. The CRISPR complex of the invention has a wide variety of utilities including modifying (e.g., deleting, inserting, translocating, inactivating, activating) a target polynucleotide in a multiplicity of cell types in various tissues and organs, e.g., immune cells. As such the CRISPR complex of the invention has a broad spectrum of applications in modeling of multiple genetic, or tissue-specific mutations, and hence gene therapy, drug discovery, drug screening, disease diagnosis, and prognosis.

It will be appreciated that in the present methods, where the non-human transgenic Cas9 organism is multicellular, e.g., an animal or plant, the modification may occur ex vivo or in vitro, for instance in a cell culture and in some instances not in vivo. In other embodiments, it may occur in vivo. In an aspect, the invention provides a method of modifying an organism or a non-human organism by manipulation of a target sequence in a genomic locus of interest comprising: Delivering, e.g., via particle(s) or nanoparticle(s) or vector(s) (e.g., viral vector, e.g., AAV, adenovirus, lentivirus) a non-naturally occurring or engineered composition. The composition can comprise: A) I. RNA(s) having polynucleotide sequence(s), e.g., a CRISPR-Cas system chimeric RNA (chiRNA) having polynucleotide a sequence, wherein the polynucleotide sequence comprises: (a) a guide sequence capable of hybridizing to a target sequence in a eukaryotic cell, (b) a tracr mate sequence, and (c) a tracr sequence; wherein (a), (b) and (c) are arranged in a 5′ to 3′ orientation. The composition can also comprise A) II. a polynucleotide sequence encoding a CRISPR enzyme advantageously comprising at least one or more or two or more nuclear localization sequences. When transcribed, the tracr mate sequence hybridizes to the tracr sequence and the guide sequence directs sequence-specific binding of a CRISPR complex to the target sequence, wherein the CRISPR complex comprises the CRISPR enzyme complexed with (1) the guide sequence that is hybridizable to the target sequence, and (2) the tracr mate sequence that is hybridizable to the tracr sequence. The polynucleotide sequence encoding a CRISPR enzyme is DNA or RNA. When the Cas9 is already present in the cell, e.g., through the cell having already been provided (A) II. or through the cell expressing Cas9, e.g., through the cell having been transformed to express Cas9, e.g., Cas9 is expressed constitutively or conditionally or inducibly—for instance when the cell is part of or from a non-human transgenic eukaryote, e.g., animal, mammal, primate, rodent, etc. as herein discussed—then (A) I. is provided as the CRISPR complex is formed in situ or in vivo. In an aspect, the invention provides a method of modifying an organism or a non-human organism by manipulation of a target sequence in a genomic locus of interest comprising: Delivering, e.g., via particle(s) or nanoparticle(s) or vector(s) (e.g., viral vector, e.g., AAV, adenovirus, lentivirus) a non-naturally occurring or engineered composition. The composition can comprise (B) I. polynucleotides comprising: (a) a guide sequence capable of hybridizing to a target sequence in a eukaryotic cell, and (b) at least one or more tracr mate sequences. The composition can also comprise (B) II. a polynucleotide sequence comprising a tracr sequence. The composition can also comprise (B) III. a polynucleotide sequence encoding a CRISPR enzyme advantageously comprising at least one or more or two or more nuclear localization sequences. When transcribed, the tracr mate sequence hybridizes to the tracr sequence and the guide sequence directs sequence-specific binding of a CRISPR complex to the target sequence. The CRISPR complex comprises the CRISPR enzyme complexed with (1) the guide sequence that is hybridizable to the target sequence, and (2) the tracr mate sequence that is hybridizable to the tracr sequence, and the polynucleotide sequence encoding a CRISPR enzyme is DNA or RNA. When the Cas9 is already present in the cell, e.g., through the cell having already been provided (B) III. or through the cell expressing Cas9, e.g., through the cell having been transformed to express Cas9, e.g., Cas9 is expressed constitutively or conditionally or inducibly—for instance when the cell is part of or from a non-human transgenic eukaryote, e.g., animal, mammal, primate, rodent, etc. as herein discussed—then (B) I. and (B) II. are provided as the CRISPR complex is formed in situ or in vivo. Accordingly, components I and II or I, II and III or the foregoing embodiments can be delivered separately; for instance, in embodiments involving components I, II and III, components I and II can be delivered together, while component II can be delivered separately, e.g., prior to components I and II, so that the cell or eukaryote expresses Cas9. It will be further appreciated that heretofore it could not be expected that multiple, specific mutations, especially in the numbers herein discussed, e.g., 3-50 or more, or 3, 16, 32, 48 or 50 or more, could be able to be achieved. In undertaking embodiments of the invention, the Applicants have indeed divined that such multiple mutations are present in significant numbers of cells of the non-human eukaryote. For instance, it was surprising and unexpected that cells and tumors of the non-human transgenic organisms of the invention could have multiple mutations of the delivered RNA(s) (sgRNAs); indeed, all of the multiple mutations.

In some embodiments the invention comprehends delivering a CRISPR enzyme comprising delivering to a cell mRNA encoding the CRISPR enzyme, e.g., via nanoparticle complex(es). In some of these methods the CRISPR enzyme is a Cas9. In certain preferred embodiments the Cas9 enzyme is constitutively present, e.g., through knock-in. Thus, in a preferred embodiment of the invention, the Cas9 enzyme is constitutively present in vivo (e.g., a non-human transgenic eukaryote, animal, mammal, primate, rodent, etc.) or ex vivo (cells comprising a vector containing nucleic acid molecule(s) for in vivo expression of the Cas9). The CRISPR enzyme is a type I or III CRISPR enzyme, preferably a type II CRISPR enzyme. This type II CRISPR enzyme may be any Cas enzyme. A preferred Cas enzyme may be identified as Cas9 as this can refer to the general class of enzymes that share homology to the biggest nuclease with multiple nuclease domains from the type II CRISPR system. Most preferably, the Cas9 enzyme is from, or is derived from, SpCas9 or SaCas9. It will be appreciated that SpCas9 or SaCas9 are those from or derived from S. pyogenes or S. aureus Cas9. By derived, Applicants mean that the derived enzyme is largely based, in the sense of having a high degree of sequence homology with, a wildtype enzyme, but that it has been mutated (modified) in some way as described herein It will be appreciated that the terms Cas and CRISPR enzyme are generally used herein interchangeably, unless otherwise apparent. The Cas enzyme can be for instance any naturally-occurring bacterial Cas9 as well as any chimaeras, mutants, homologs or orthologs. Many of the residue numberings used herein refer to the Cas9 enzyme from the type II CRISPR locus in Streptococcus pyogenes (annotated alternatively as SpCas9 or spCas9). However, it will be appreciated that this invention includes many more Cas9s from other species of microbes, e.g., orthologs of SpCas9, or Cas9s derived from microbes in addition to S. pyogenes, e.g., SaCas9 derived from S. aureus, St1Cas9 derived from S. thermophilus and so forth. The skilled person will be able to determine appropriate corresponding residues in Cas9 enzymes other than SpCas9 by comparison of the relevant amino acid sequences. Thus, where a specific amino acid replacement is referred to using the SpCas9 numbering, then, unless the context makes it apparent this is not intended to refer to other Cas9 enzymes, the disclosure is intended to encompass corresponding modifications in other Cas9 enzymes. Cas9 orthologs typically share the general organization of 3-4 RuvC domains and a HNH domain. The 5′ most RuvC domain cleaves the non-complementary strand, and the HNH domain cleaves the complementary strand. All notations are in reference to the guide sequence. The catalytic residue in the 5′ RuvC domain is identified through homology comparison of the Cas9 of interest with other Cas9 orthologs (from S. pyogenes type II CRISPR locus, S. thermophilus CRISPR locus 1, S. thermophilus CRISPR locus 3, and Franciscilla novicida type II CRISPR locus), and the conserved Asp residue (D10) is mutated to alanine to convert Cas9 into a complementary-strand nicking enzyme. Similarly, the conserved His and Asn residues in the HNH domains are mutated to Alanine to convert Cas9 into a non-complementary-strand nicking enzyme. In some embodiments, both sets of mutations may be made, to convert Cas9 into a non-cutting enzyme. Thus, the Cas9 may comprise one or more mutations and may be used as a generic DNA binding protein with or without fusion to a functional domain. The mutations may be artificially introduced mutations or gain- or loss-of-function mutations. The mutations may include but are not limited to mutations in one of the catalytic domains (e.g., D10 and H840) in the RuvC and HNH catalytic domains respectively; or the CRISPR enzyme can comprise one or more mutations selected from the group consisting of D10A, E762A, H840A, N854A, N863A or D986A and/or one or more mutations in a RuvC1 or HNH domain of the CRISPR enzyme or has a mutation as otherwise as discussed herein. In one aspect of the invention, the Cas9 enzyme may be fused to a protein, e.g., a TAG, and/or an inducible/controllable domain such as a chemically inducible/controllable domain. The Cas9 in the invention may be a chimeric Cas9 proteins; e.g., a Cas9 having enhanced function by being a chimera. Chimeric Cas9 proteins may be new Cas9 containing fragments from more than one naturally occurring Cas9. These may comprise fusions of N-terminal fragment(s) of one Cas9 homolog with C-terminal fragment(s) of another Cas9 homolog. The Cas9 can be delivered into the cell in the form of mRNA. The expression of Cas9 can be under the control of an inducible promoter.

The tracrRNA and direct repeat sequences can be mutant sequences or the invention can encompass RNA of the CRISPR-Cas system that includes mutant chimeric guide sequences that allow for enhancing performance of these RNAs in cells. A suitable promoter, such as the Pol III promoter, such as a U6 promoter, can be added onto the guide RNA that is advantageously delivered via AAV or particle or nanoparticle. Aspects of the invention also relate to the guide RNA being transcribed in vitro or ordered from a synthesis company and directly transfected. Expression of RNA(s), e.g., guide RNAs or sgRNA under the control of the T7 promoter driven by the expression of T7 polymerase in the cell is also envisioned. In an advantageous embodiment, the cell is a eukaryotic cell. In a preferred embodiment the eukaryotic cell is a human cell. In a more preferred embodiment the cell is a patient-specific cell, e.g., a cell in which 3-50 or more mutations associated or correlated with a patient's genetic disease, e.g., an immune or infectious disease, are expressed in the cell, e.g., via Cas9 being present in the cell and RNA(s) for such mutations delivered to the cell (e.g., any whole number between 3 and 50 of mutations, with it noted that in some embodiments there can be up to 16 different RNA(s), e.g., sgRNAs, e.g., each having its own a promoter, in a vector, such as lentivirus, and that when each sgRNA does not have its own promoter, there can be twice to thrice that amount of different RNA(s), e.g., sgRNAs, e.g., 32 or even 48 different guides delivered by one vector, whereby the CRISPR-Cas complexes result in the cells having the mutations and the cells and the eukaryote, e.g., animal, containing the cells being a model for the patient's genetic disease.

A codon-optimized sequence can be a sequence optimized for a eukaryote, or for specific organs or cell types such as immune cells (e.g., T cells). It will be appreciated that where reference is made to a polynucleotide, where that polynucleotide is RNA and is said to ‘comprise’ a feature such as a tracr mate sequence, the RNA sequence includes the feature. Where the polynucleotide is DNA and is said to comprise a feature such as a tracr mate sequence, the DNA sequence is or can be transcribed into the RNA that comprises the feature at issue. Where the feature is a protein, such as the CRISPR enzyme, the DNA or RNA sequence referred to is, or can be, translated (and in the case of DNA transcribed first). Furthermore, in cases where an RNA encoding the CRISPR enzyme is provided to a cell, it is understood that the RNA is capable of being translated by the cell into which it is delivered. By manipulation of a target sequence, Applicants mean the alteration of the target sequence, which may include the epigenetic manipulation of a target sequence. This epigenetic manipulation may be of the chromatin state of a target sequence, such as by modification of the methylation state of the target sequence (i.e. addition or removal of methylation or methylation patterns or CpG islands), histone modification, increasing or reducing accessibility to the target sequence, or by promoting 3D folding. It will be appreciated that where reference is made to a method of modifying an organism or mammal including human or a non-human mammal or organism by manipulation of a target sequence in a genomic locus of interest, this may apply to the organism (or mammal) as a whole or just a single cell or population of cells from that organism (if the organism is multicellular). Applicants envisage, inter alia, a single cell or a population of cells and these may preferably be modified ex vivo and then re-introduced, e.g., transplanted to make transgenic organisms that express Cas9 in certain cells. The invention in some embodiments comprehends a method of modifying a eukaryote, such as a Cas9 transgenic eukaryote comprising delivering, e.g., via vector(s) and/or particle(s) and/or nanoparticles a non-naturally occurring or engineered composition. The composition comprises: I. a first regulatory element operably linked to (a) a first guide sequence capable of hybridizing to a first target sequence, and (b) at least one or more tracr mate sequences, II. a second regulatory element operably linked to (a) a second guide sequence capable of hybridizing to a second target sequence, and (b) at least one or more tracr mate sequences, III. a third regulatory element operably linked to (a) a third guide sequence capable of hybridizing to a third target sequence, and (b) at least one or more tracr mate sequences, and IV. a fourth regulatory element operably linked to a tracr sequence. There can be additional regulatory element(s) operably linked to additional guide sequence(s). Optionally, the composition can involve V. a fifth regulatory element operably linked to an enzyme-coding sequence encoding a CRISPR enzyme (e.g., for establishing the Cas9 transgenic eukaryote). Components I, II, III and IV (as well as any other regulatory element(s) linked to additional guide sequence(s)) are located on the same or different vectors and/or particles and/or nanoparticles of the system. When transcribed, the tracr mate sequence hybridizes to the tracr sequence and the first, second and the third guide sequences direct sequence-specific binding of a first, second and a third CRISPR complexes to the first, second and third target sequences respectively, wherein the first CRISPR complex comprises the CRISPR enzyme complexed with (1) the first guide sequence that is hybridizable to the first target sequence, and (2) the tracr mate sequence that is hybridizable to the tracr sequence, wherein the second CRISPR complex comprises the CRISPR enzyme complexed with (1) the second guide sequence that is hybridizable to the second target sequence, and (2) the tracr mate sequence that is hybridizable to the tracr sequence, and wherein the third CRISPR complex comprises the CRISPR enzyme complexed with (1) the third guide sequence that is hybridizable to the third target sequence, and (2) the tracr mate sequence that is hybridizable to the tracr sequence, whereby in a Cas9 transgenic eukaryote or cell thereof, at least three (3) mutations may be induced. The invention also provides a vector system as described herein. The system may comprise one, two, three or four different vectors; and the system may comprise one, two, three or four different nanoparticle complex(es) delivering the component(s) of the system. Components I, II, III and IV may thus be located on one, two, three or four different vectors, and may be delivered by one, two, three or four different particle or nanoparticle complex(es) or AAVs or components I, II, III and IV can be located on same or different vector(s)/particle(s)/nanoparticle(s), with all combinations of locations envisaged. And complexes that target immune tissue or cells are advantageous.

In some methods, a target polynucleotide can be inactivated to effect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein or microRNA or pre-microRNA transcript is not produced. In certain embodiments, the target sequence is flanked or followed, at its 3′ end, by a PAM suitable for the CRISPR enzyme, typically a Cas and in particular a Cas9. For example, a suitable PAM is 5′-NRG or 5′-NNGRR for SpCas9 or SaCas9 enzymes (or derived enzymes), respectively.

Delivery can be in the form of a vector which may be a plasmid or other nucleic acid molecule form, especially when the delivery is via a nanoparticle complex; and the vector also can be viral vector, such as a herpes, e.g., herpes simplex virus, lenti- or baculo- or adeno-viral or adeno-associated viral vectors, but other means of delivery are known (such as yeast systems, microvesicles, gene guns/means of attaching vectors to gold nanoparticles) and are provided, especially as to those aspects of the complex not delivered via a nanoparticle complex. A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell; and advantageously the complex or a component thereof is delivered via nanoparticle complex(es). Also envisaged is a method of delivering the present CRISPR enzyme comprising delivering to a cell mRNA encoding the CRISPR enzyme; and advantageously the complex or a component thereof has been delivered via nanoparticle complex(es). It will be appreciated that in certain embodiments the CRISPR enzyme is truncated, and/or comprised of less than one thousand amino acids or less than four thousand amino acids, and/or is a nuclease or nickase, and/or is codon-optimized, and/or comprises one or more mutations, and/or comprises a chimeric CRISPR enzyme, and/or the other options as herein discussed. When not delivering via a nanoparticle complex, AAV is a preferred vector. In certain embodiments, multiple RNA(s) or guide RNAs or sgRNAs formulated in one or more delivery vehicles (e.g., where some guide RNAs are provided in a vector and others are formulated in nanoparticles); and these may be provided alone (e.g., when Cas9 is already in a cell) or with a Cas9 delivery system. In certain embodiments, the Cas9 is also delivered in a nanoparticle formulation. In certain instances the RNA(s) or guide RNA or sgRNA-vector and/or particle and/or nanoparticle formulation(s) and the Cas9 vector and/or particle and/or nanoparticle formulation(s) may be delivered separately or may be delivered substantially contemporaneously (i.e., co-delivery). Sequential delivery could be done at separate points in time, separated by days, weeks or even months. And as Cas9 is advantageously present in a transgenic organism in the practice of the invention, e.g., through being constitutively or conditionally or inducibly present, sequential delivery can include initially administering or delivering the Cas9 vector and/or particle and/or nanoparticle formulation(s) to cells that give rise to the non-human Cas9 transgenic eukaryote, and thereafter, at a suitable time in the life of the transgenic eukaryote, administering the RNA(s) or guide RNA or sgRNA-vector and/or particle and/or nanoparticle formulation(s), e.g., so as to give rise to one or more, advantageously 3-50 mutations in the transgenic eukaryote (e.g., any whole number between 3 and 50 of mutations, with it noted that in some embodiments there can be up to 16 different RNA(s), e.g., sgRNAs each having its own a promoter, in a vector, such as AAV, and that when each sgRNA does not have its own promoter, there can be twice to thrice that amount of different RNA(s), e.g., sgRNAs, e.g., 32 or even 48 different guides delivered by one vector). In certain embodiments, vector (e.g., AAV, adenovirus, lentivirus) and/or particle and/or nanoparticle formulations comprising one or more RNA(s) e.g. guide RNAs or sgRNA are adapted for delivery in vitro, ex vivo or in vivo in the context of the CRISPR-Cas system, e.g., so as to form CRISPR-Cas complexes in vitro, ex vivo or in vivo, to different target genes, different target cells or different target different tissues/organs, with different target genes. Multiplexed gene targeting using nanoparticle formulations comprising one or more guide RNAs are also envisioned. In an embodiment, a nanoparticle formulation comprising one or more components of the CRISPR-Cas system is provided. In an embodiment, a RNA(s) or gRNA or sgRNA-nanoparticle formulation comprising one or more guide RNAs or sgRNA is provided. In certain embodiments, a composition comprising a nanoparticle formulation comprising one or more components of the CRISPR-Cas system is provided. In certain embodiments, a composition, e.g., a pharmaceutical or veterinary composition, comprising a vector (e.g., AAV, adenovirus, lentivirus) and/or particle and/or nanoparticle formulation comprising one or more components of the CRISPR-Cas system and/or nucleic acid molecule(s) coding therefor, advantageously with such nucleic acid molecule(s) operably linked to promoter(s) is provided. Accordingly, in certain embodiments, it may be useful to deliver the RNA(s) or guide RNA or sgRNA, e.g., vector and/or particle and/or nanoparticle formulations separately from the Cas9 or nucleic acid molecule(s) coding therefor. A dual-delivery system is envisaged such that the Cas 9 may be delivered via a vector and the RNA(s), e.g., guide RNAs or sgRNA are/is provided in a particle or nanoparticle formulation, for example, first Cas9 vector is delivered via a vector system followed by delivery of sgRNA-nanoparticle formulation. Vectors may be considered in the broadest light as simply any means of delivery, rather than specifically viral vectors.

In one aspect, the present invention provides a Cas9 transgenic eukaryote, e.g., mouse. In certain preferred embodiments, the Cas 9 transgenic eukaryote, e.g., mouse comprises a Cas9 transgene knocked into the Rosa26 locus. In one aspect, the present invention provides a Cas9 transgenic eukaryote, e.g., mouse wherein Cas9 transgene is driven by the ubiquitous CAG promoter thereby providing for constitutive expression of Cas9 in all tissues/cells/cell types of the mouse. In one aspect, the present invention provides a Cas9 transgenic eukaryote, e.g., mouse wherein the Cas9 transgene driven by the ubiquitous CAG promoter further comprises a Lox-Stop-polyA-Lox (LSL) cassette (Rosa26-LSL-Cas9 mouse) thereby rendering Cas9 expression inducible by the Cre recombinase. In one aspect, the present invention provides a constitutive Cas9 expressing eukaryote, e.g., mouse line obtained by crossing of the Rosa26-LSL-Cas9 mouse with a beta-actin-Cre eukaryote, e.g., mouse line. In certain embodiments, progeny(ies) derived from said Cas9 expressing eukaryote, e.g., mouse line may be successfully bred over at least five generations without exhibiting increased levels of genome instability or cellular toxicity. In one aspect, the present invention provides a modular viral vector construct comprising a plurality of sgRNAs driven by a single RNA polymerase III promoter (e.g., U6), wherein the sgRNAs are in tandem, or where each of the sgRNAs in driven by one RNA polymerase III promoter. In one aspect, the present invention provides a modular viral vector construct comprising one or more cassettes expressing Cre recombinase, a plurality of sgRNAs to guide Cas9 cutting, and a HDR template to model the dynamics of a complex pathological disease or disorder involving two or more genetic elements simultaneously using a single vector construct. In certain embodiments, the modular viral vector construct comprises one or more cassettes expressing Cre recombinase, a plurality of sgRNAs to guide Cas9 cutting, and a Homology Directed Repair (HDR) template to introduce specific gain-of-function mutations or precise sequence substitution in target loci. In one aspect, the present invention provides a method for simultaneously introducing multiple mutations ex vivo in a tissue, organ or a cell line, or in vivo in the same animal comprising delivering a single viral vector construct, wherein the viral vector construct comprises one or more cassettes expressing Cre recombinase, a plurality of sgRNAs to guide Cas9 cutting, and a HDR template for achieving targeted insertion or precise sequence substitution at specific target loci of interest. In one aspect, the present invention provides a method for delivering ex vivo or in vivo of any of the constructs disclosed herein using a viral vector.

In certain preferred embodiments, lentivirus is used for delivery to hematopoietic and/or immune cells. In one aspect, the present invention provides a method for ex vivo and/or in vivo genome editing comprising delivering any of the above modular viral vector constructs, which comprise one or more cassettes expressing Cre recombinase, a plurality of sgRNAs to guide Cas9 cutting, and a HDR template, into a Cas9 transgenic mouse (e.g., Rosa26-LSL-Cas9). In certain embodiments, the viral vector is a lentivirus. It can be appreciated that using the novel CRISPR-Cas9 tools disclosed herein, Cas9 transgenic non-human eukaryote, e.g., animal model with multiple mutations in any number of loci can be envisioned and are within the scope of the present invention. Such uses are within the scope of the present invention. In one aspect, the present invention provides a method of treating or inhibiting the development of a genetic disease in a subject in need thereof, comprising providing individualized or personalized treatment (or an individualized or personalized model or patient specific-modeling) comprising: delivering RNA(s), e.g., sgRNA, that targets a genetic locus correlated or associated with the genetic disease to a Cas9 non-human transgenic eukaryote (e.g., animal, mammal, primate, rodent, fish etc. as herein discussed), e.g., via vector such as AAV, adenovirus, lentivirus, or particle(s) or nanoparticle(s), whereby mutation(s), advantageously a plurality, e.g., 3-50 mutations (e.g., any whole number between 3 and 50 of mutations, with it noted that in some embodiments there can be up to 16 different RNA(s), e.g., sgRNAs each having its own a promoter, in a vector, such as lentivirus, and that when each sgRNA does not have its own promoter, there can be twice to thrice that amount of different RNA(s), e.g., sgRNAs, e.g., 32 or even 48 different guides delivered by one vector), are induced in the eukaryote and the eukaryote is a model for the disease; and obtaining and/or extrapolating data from the Cas9 non-human transgenic eukaryote to humans to provide individualized or personalized treatment. The obtaining and/or extrapolating data can be subjecting the eukaryote to putative treatment(s) and/or therapy(ies), e.g., gene therapy, ascertaining whether such putative treatment(s) and/or therapy(ies) give rise to remission or treatment or alleviation or remission of the disease, and if so, then administering in dosing scaled to a 70 kg individual or subject, the putative treatment(s) and/or therapy(ies). The invention thus allows for one to ascertain whether a particular treatment and/or therapy may be effective as to a particular individual's disease.

In certain aspects the invention provides vector(s), particle(s) or nanoparticle(s) containing nucleic acid molecule(s), whereby in vivo in a eukaryotic cell containing or conditionally or inducibly expressing Cas9: the vector(s) express(es) a plurality of RNAs to guide the Cas9 and delivers donor templates (e.g., HDR templates), and optionally in the event Cas9 is conditionally or inducibly expressed in the cell that which induces Cas9, e.g., Cre recombinase; whereby a plurality of specific mutations or precise sequence substitutions in a plurality of target loci are introduced. The vector(s) can be a viral vector such as lentivirus, adenovirus, or adeno-associated virus (AAV), e.g., AAV6 or AAV9. The Cas9 can be from S. thermophiles, S. aureus, or S. pyogenes. The eukaryotic cell can comprise a Cas9 transgene is functionally linked to a constitutive promoter, or a tissue specific promoter, or an inducible promoter; and, the eukaryotic cell can be part of a non-human transgenic eukaryote, e.g., a non-human mammal, primate, rodent, mouse, rat, rabbit, canine, dog, cow, bovine, sheep, ovine, goat, pig, fowl, poultry, chicken, fish, insect or arthropod; advantageously a mouse. The isolated eukaryotic cell or the non-human transgenic eukaryote can express an additional protein or enzyme, such as Cre; and, the expression of Cre can be driven by coding therefor functionally or operatively linked to a constitutive promoter, or a tissue specific promoter, or an inducible promoter.

The RNAs to guide Cas9 can comprise CRISPR RNA and transactivating (tracr) RNA. The tracr mate and the tracr sequence can connected to form a transactivating (tracer) sequence. The tracr mate and the tracr sequence to form a single guide RNA (sgRNA). Indeed, it is advantageous that the RNAs to guide Cas9 can comprise chimeric single guide RNA (sgRNA). The tracr sequence and tracr mate sequence along the length of the shorter of the two when optimally aligned can be about or more than about 25%, 30%, 40%, 50%, 60%, 70%, 80%, 90%, 95%, 97.5%, 99%, or higher. The tracr sequence can be about or more than about 5, 6, 7, 8, 9, 10, 11, 12, 13, 14, 15, 16, 17, 18, 19, 20, 25, 30, 40, 50, or more nucleotides in length. The degree of complementarity between a guide sequence and its corresponding target sequence can be about or more than about 50%, 60%, 75%, 80%, 85%, 90%, 95%, 97.5%, 99%, or 100%. A guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. A guide or RNA or sgRNA can be less than about 75, 50, 45, 40, 35, 30, 25, 20, 15, 12, or fewer nucleotides in length.

The vector(s) can include the regulatory element(s), e.g., promoter(s). The vector(s) can comprise at least 3 or 8 or 16 or 32 or 48 or 50 RNA(s) (e.g., sgRNAs), such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s) (e.g., sgRNAs); and, when a single vector provides for more than 16 RNA(s) (e.g., sgRNAs), one or more promoter can drive expression of more than one of the RNA(s) (e.g., sgRNAs), e.g., when there are 32 RNA(s) (e.g., sgRNAs), each promoter can drive expression of two RNA(s) (e.g., sgRNAs), and when there are 48 RNA(s) (e.g., sgRNAs), each promoter can drive expression of three RNA(s) (e.g., sgRNAs). By simple arithmetic and well-established cloning protocols and the teachings in this disclosure one skilled in the art can readily practice the invention as to the RNA(s) (e.g., sgRNA(s) for a suitable exemplary vector such as AAV, and a suitable promoter such as the U6 promoter, e.g., U6-sgRNAs. For example, the packaging limit of AAV is ˜4.7 kb. The length of a single U6-sgRNA (plus restriction sites for cloning) is 361 bp. Therefore, the skilled person can readily fit about 12-16, e.g. 13 U6-sgRNA cassettes in a single vector. This can be assembled by any suitable means, such as a golden gate strategy used for TALE assembly (http://www.genome-engineering.org/taleffectors/). The skilled person can also use a tandem guide strategy to increase the number of U6-sgRNAs by approximately 1.5 times, e.g., to increase from 12-16, e.g., 13 to approximately 18-24, e.g., about 19 U6-sgRNAs. Therefore, one skilled in the art can readily reach approximately 18-24, e.g., about 19 promoter-RNAs, e.g., U6-sgRNAs in a single vector, e.g., an AAV vector. A further means for increasing the number of promoters and RNAs, e.g., sgRNA(s) in a vector is to use a single promoter (e.g., U6) to express an array of RNAs, e.g., sgRNAs separated by cleavable sequences. And an even further means for increasing the number of promoter-RNAs, e.g., sgRNAs in a vector is to express an array of promoter-RNAs, e.g., sgRNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short, www.nature.com/mt/journal/v16/n9/abs/mt2008144a.html). Accordingly, from the knowledge in the art and the teachings in this disclosure the skilled person can readily make and use vector(s), e.g., a single vector, expressing multiple RNAs or guides or sgRNAs under the control or operatively or functionally linked to one or more promoters—especially as to the numbers of RNAs or guides or sgRNAs discussed herein, without any undue experimentation.

The RNA(s), e.g., sgRNA(s) can be functionally or operatively linked to regulatory element(s) and hence the regulatory element(s) drive expression. The promoter(s) can be constitutive promoter(s) and/or inducible promoter(s) and/or tissue specific promoter(s). The promoter can be selected from the group consisting of RNA polymerases, poly I, poly II, poly III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Each sgRNA can be driven by an independent promoter, e.g., U6 promoter. The vector can be viral vector, e.g., lentivirus. Each of the sgRNAs can target a different genetic locus associated with a multigenic disease or disorder. The invention also comprehends a method for introducing multiple mutations ex vivo in a tissue, organ or a cell line comprising Cas9-expressing eukaryotic cell(s), or in vivo in a transgenic non-human mammal having cells that express Cas9, comprising delivering to cell(s) of the tissue, organ, cell or mammal the vector as herein-discussed. The method can comprise delivering to cells of the transgenic non-human mammal, and the transgenic non-human mammal is a transgenic mouse having cells that express Cas9, e.g., a mouse that has had a Cas9 transgene knocked into the Rosa26 locus. The Cas9 transgene can further comprise a Lox-Stop-polyA-Lox(LSL) cassette thereby rendering Cas9 expression inducible by Cre recombinase. This method can comprise delivering to cells of the transgenic non-human mammal the vector, and the transgenic non-human mammal is a transgenic mouse having cells that express Cas9, e.g., a mouse that has had a Cas9 transgene knocked into the Rosa26 locus; and, the Cas9 transgene can further comprise a Lox-Stop-polyA-Lox(LSL) cassette thereby rendering Cas9 expression inducible by Cre recombinase.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. Nothing herein is intended as a promise.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1G. depicts that cancer cell lines with homozygous loss of MTAP are selectively sensitive to suppression of PRMT5 or WDR77 complex. a. Frequency of MTAP deletion for selected cancers are shown. Data was obtained from the cBioPortal for Cancer Genomics (www.cbioportal.org). MPNST, malignant peripheral nerve sheath tumor; GBM, glioblastoma; DLBCL, diffuse large B-cell lymphoma. b. Identification of shRNAs with strong differential effect on 216 cancer cell lines with and without MTAP deletion. Point biserial correlation coefficients are plotted against Wilcoxon two-class comparison test p-values for 50,529 shRNAs. Two shRNAs with strong differential effects on viability for MTAP⁻ lines are identified. c. Cell lines sensitivities to GCCCAGTTTGAGATGCCTTAT_PRMT5 (SEQ ID NO: 1) and GCAAAGTGAAGTCTTTGTCTT_WDR77 (SEQ ID NO: 2) shRNAs are highly correlated, with cell lines lacking MTAP (shown in red) being more sensitive. d. Log2(fold) of shPRMT5 #1 depletion is plotted for cell lines with the indicated genotypes. Median with upper and lower 25^{th} percentiles are shown. e. Selective sensitivity of cell lines lacking MTAP to the genetic knockdown of PRMT5 activity is supported by at least two hairpins for four members of the complex. Pearson correlation test p-values for the top-scoring shRNAs are plotted against p-values for the second best-scoring shRNAs targeting the same gene. Constitutive members of the complex PRMT5 and WDR77 are shown in red. Mutually exclusive substrate adaptors CLNS1A and RIOK1 are shown in orange. Genes not expressed in 90% of tumors lacking MTAP are shown in grey. f. Log2(fold) of shPRMT5 #1 depletion is plotted for all 216 cell lines (left) and for lines from the indicated lineages (lung_NSC, non-small cell lung cancer; AML, acute myeloid leukemia). g. Log2(fold) depletion for the indicated shRNAs is shown for all 275 cell lines from the validation cohort.

FIG. 2A-2C. depicts cells with MTAP loss are more sensitive to knockdown of PRMT5 and WDR77 than isogenic MTAP-reconstituted cells. a. Protein lysates were harvested from LU99 or H647 cells and from MTAP-reconstituted LU99 or H647 cells 5 days after lentiviral transduction with the indicated shRNAs (control shRNA or shRNA against PRMT5 or WDR77). Lysates were fractionated by SDS-PAGE, and immunoblotting was performed with the indicated antibodies. b. LU99 or H647 cells and MTAP-reconstituted LU99 or H647 cells were transduced with lentivirus harboring the indicated shRNAs and stained with crystal violet after 10 to 18 days. Media change was performed every 3 days. c. Quantitation of crystal violet uptake by cells transduced with shRNAs against PRMT5 or WDR77 (normalized to control shRNA for each cell line). Mean and standard error of 3-4 replicates are shown. The experiment was repeated 2-3 times for each of the four cell line pairs. **p<0.01; *p<0.05 by Student's t-test.

FIG. 3A-3F. depicts that intracellular MTA is increased in cells with MTAP loss and correlates with sensitivity to PRMT5 suppression. a. Relative abundance of 56 profiled metabolites was compared for cell extracts from four isogenic cell line pairs. Fold-change in relative abundance of each metabolite with MTAP reconstitution is shown for each isogenic pair. Results reflect the mean of 2 independent experiments with 3 replicates per cell line. Findings for MTA (methylthioadenosine) are indicated with an asterisk. b. Representative extracted ion chromatograms (XICs) from LC-MS analysis of cellular extracts from SF-172 (left) or MTAP-reconstituted SF-172 (right) demonstrating a peak corresponding to MTA. RT, retention time; m/z, mass-to-charge ratio. c. Relative abundance of MTA from cellular extracts is displayed. Mean and standard error of 3 biological replicates are shown. The entire experiment was performed twice with similar findings for all four cell lines. *p<0.01 by Student's t-test. d. Correlation of metabolite levels with MTAP status is shown. Point biserial correlation coefficients are plotted against Wilcoxon two-class comparison test p-values for 73 metabolites profiled across 40 MTAP+ and MTAP− cell lines. e. Relative abundance of MTA from MTAP+(n=21) and MTAP− (n=19) cell lines from various lineages is shown. Mean of 3 biological replicates is shown for each cell line. Median with upper and lower 25^{th} percentiles are shown. f. Correlation of intracellular MTA levels with sensitivity to PRMT5 depletion is shown. shPRMT5 sensitivity data from the screening and validation studies were normalized and combined using modified z-scores. Z-scores are plotted against relative intracellular abundance of MTA for the 40 assayed cell lines. Spearman rank correlation p-value is shown.

FIG. 4A-4D. depicts the pharmacological inhibition of PRMT5. a. Cells were exposed to DMSO or 200 μM MTA for 48 hours. Lysates were harvested and fractionated by SDS-PAGE. Immunoblotting was performed with the indicated antibodies [vinculin or antibodies recognizing symmetric or asymmetric di-methyl arginine motifs (sDMA and aDMA, respectively)]. Molecular weight is indicated on the right in kDa. b. Dendrogram and heat map indicating relative sensitivity of 31 histone methyltransferases to inhibition by MTA as determined by radioisotope filter binding assay. c,d, Relative cell viability IC₅₀ (normalized to MTAP+ cells for each line) for cells treated with EPZ015666 (top) or MTA (bottom), for isogenic cell lines derived from (C) MTAP-expressing parental cell lines or (D) MTAP− parental cell lines. Mean IC₅₀ and 95% confidence interval of 6 replicates are shown for each cell line. Each experiment was performed twice for each cell line. *=p<0.05 by two-tailed Student's t-test (vs. MTAP+ cells).

FIG. 5A-5I. depicts that MTAP− tumor cells are sensitive to enzymatic inhibition of PRMT5. a, PRMT5i structure. b, c, Western blot analysis of vinculin (VCL), PRMT5 product sDMA and type-1 PRMT (PRMT1, 3, 4, 6, 8) product aDMA, in lysates from (a) MTAP− BFTC909 tumor cells and (b) MTAP+ NCIH2030 tumor cells, treated with the indicated doses of PRMT5i or 100 μM MTA for 4 days. d-g, 12 day viability analysis of MTAP− (SU.86.86, MIAPACA, NCIH647, LU99, BFTC909, JHOS2) or MTAP+(KP2, HCC827, NCIH2030, 7860, OVTOKO) tumor cell lines derived from (d) pancreatic cancer, (e) lung cancer, (f) renal cell carcinoma, or (g) ovarian cancer, following treatment with indicated doses of PRMT5i. h, Dot plot showing 12 day cell viability IC50s for PRMT5i, from MTAP− or MTAP+ cell lines shown in panels d-g. *=p-value<0.005. i, A hypothesis by which MTAP loss may lead to inhibition of PRMT5 activity and reduced cell viability in combination with pharmacologic inhibition of PRMT5.

FIG. 6A-6D. depicts immunoblotting that confirms on-target activity of shRNAs against PRMT5 and WDR77. a. Protein lysates were harvested from SF-172 glioma cells 5 days after lentiviral transduction with the indicated shRNAs [control shRNA against Lac Z (shLac Z) or shRNAs against PRMT5 or WDR77]. Lysates were fractionated by SDS-PAGE, and immunoblotting was performed with the indicated antibodies. b. As in a, except for SU.86.86 cells, which are a pancreatic ductal carcinoma cell line. c. RNA was harvested from SF-172 cells 5 days after lentiviral transduction with the indicated shRNAs or with a control shRNA (shLac Z). cDNA was prepared and used as a template for real-time quantitative PCR with primers designed to amplify PRMT5, WDR77, and HPRT1 (control). Fold-change (2^{-ΔΔct}) of PRMT5 and WDR77 transcripts (compared to levels of these transcripts from cells transduced with shLac Z after normalization with HPRT1) is shown for cells transduced with the indicated shRNAs. Mean and standard error from 3 independent experiments (each with 3 biological replicates) are shown. d. As in C, except for SU.86.86 cells.

FIG. 7A-7B. depicts that cells with MTAP loss are sensitive to suppression of PRMT5 and WDR77. a. Protein lysates were harvested from SF-172 or SU.86.86 cells and from MTAP-reconstituted SF-172 or SU.86.86 cells 5 days after lentiviral transduction with the indicated shRNAs (control shRNA or shRNA against PRMT5 or WDR77). Lysates were fractionated by SDS-PAGE, and immunoblotting was performed with the indicated antibodies. b. SF-172 or SU.86.86 cells and MTAP-reconstituted SF-172 or SU.86.86 cells were transduced with lentivirus harboring the indicated shRNAs and stained with crystal violet after 10 to 18 days. Media change was performed every 3 days. Quantitation of crystal violet uptake is shown in FIG. 2C.

FIG. 8A-8H. depicts PRMT5i biochemical characterization. a, b, Scintillation proximity assay (SPA) measurement of PRMT5/WDR77 enzymatic activity at different substrate concentrations to determine K_{M} for PRMT5 substrates (a) Histone H4(1-21)-Lys(Biotin), and (b) SAM. c, SPA measurement of dose-response to indicated doses of PRMT5i. d, e, f, PRMT5i kinetic evaluation of histone H4(1-21)-Lys(Biotin). g, h, PRMT5i kinetic evaluation of SAM.

FIG. 9A-9E. depicts PRMT5i stability in media. a. Quantification of LCMS measurement of PRMT5i concentration in tumor cell media at 0 hours and 72 hours after incubation at 37° C., relative to fixed amount of EZH2 inhibitor GSK343 added immediately prior to each measurement. b, c. Electrospray mass spectrometry plots showing PRMT5i at LC retention time ˜1.00 minutes, and EZH2 inhibitor GSK343 at LC retention time ˜2.40 minutes, after PRMT5i had been incubated for (b) 0 hours or (c) 72 hours at 37° C. in media. d, e. Mass spectrum showing (d) PRMT5i at 0.968 minutes LC retention time and (e) GSK343 at 2.370 minutes LC retention time.

FIG. 10 depicts the crystal structure the assumed biological molecule of the human PRMT5:MEP50 Complex.

FIG. 11 depicts the crystal structure the asymmetric unit of the human PRMT5:MEP50 Complex.

FIG. 12 depicts ubiquitous expression of MTAP in normal tissues. The figure was generated using the Genotype-Tissue Expression project (GTEx) portal MTAP is ubiquitously expressed in normal tissues. The figure was generated using the Genotype-Tissue Expression project (GTEx) portal (www.gtexportal.org) and is derived from RNAseq-based expression data for 7349 tissue samples from 550 individuals (GTEx data free version phs000424.v5.p1). RPKM, reads per kilobase per million reads. Colors indicate different tissue/organ systems. The dashed line at RPKM=0.10 denotes the lower limit of expression detection above background and is derived from RNAseq-based expression data for 7349 tissue samples from 550 individuals (GTEx data free version phs000424.v5.p1). RPKM, reads per kilobase per million reads. Colors indicate different tissue/organ systems. The dashed line at RPKM=0.10 denotes the lower limit of expression detection above background.

FIG. 13A-13B. depicts sensitivity to shWDR77. a. Log2(fold) of shWDR77 #1 depletion is plotted for cell lines with the indicated genotypes. Median with upper and lower 25^{th} percentiles are shown. MTAP− cell lines (red) are generally more sensitive to WDR77 suppression than MTAP+ regardless of CDKN2A deletion status. b. Log2(fold) of shPRMT5 #1 depletion is plotted for all cell lines (left) and for lines from the indicated lineages. Globally and within individual lineages, MTAP− cells are more sensitive to WDR77 suppression than MTAP+. lung_NSC, non-small cell lung cancer; AML, acute myeloid leukemia.

FIG. 14A-14B. depicts an analysis of shared “seed” sequences support on-target activity of shRNAs against PRMT5. Differential sensitivity of MTAP− cell lines is not observed with shRNAs sharing “seed” sequences with shPRMT5 #1. a. Sensitivity to shPRMT5 #1 (SEQ ID NO: 1) is plotted against sensitivity to an shRNA targeting ALOX15 (SEQ ID NO: 35) that shares an 11 nucleotide sequence in the “seed” region. b. Sensitivity to shPRMT5 #1 (SEQ ID NO: 1) is plotted against sensitivity to an shRNA targeting ROBO4 (SEQ ID NO: 36) that shares an 8 nucleotide sequence in the “seed” region.

FIG. 15 depicts sensitivity of cancer cell lines with homozygous MTAP deletion to suppression of PRMT5 or WDR77. Log2(fold) depletion for the indicated shRNAs is shown for all cell lines from both the screening a. and validation b. cohorts. The screening cohort consists of 216 cancer cell lines (50 MTAP−; 166 MTAP+). The validation cohort consists of 275 cancer cell lines (47 MTAP−; 228 MTAP+).

FIG. 16A-16B. depicts SAM-competitive inhibition of PRMT5 by MTA. a. PRMT5 activity was measured at varying concentrations of MTA following pre-incubation with varying concentrations of SAM. b. Linear fit to points representing IC50 for MTA at varying concentrations of SAM.

FIG. 17A-17H. depicts the pharmacological inhibition of PRMT5 in isogenic cell lines. a. MTAP expression was reconstituted in MIPACA2 (MIA.), H838, and H2126 cell lines, which typically lack MTAP expression. b. MTAP was knocked out from HCC44, KP2, H2030 and H661, which typically express MTAP, using lentiCRISPR v2 with sgRNAs against MTAP. c,d. Non-normalized IC50 values for all isogenic cell line sets treated with (C) MTA or (D) EPZ015666. Error bars represent 95% CI based on calculated fit. e,f. Non-normalized IC₅₀ values for parental MTAP+ or MTAP− cell lines treated with (E) MTA or (F) EPZ015666. Mean and standard deviation are shown. g,h. Relative cell viability data used to generate IC50 values for cells treated with (E) MTA or (F) EPZ015666. Mean and standard deviation of 6 replicates are shown. Results are representative of 2 independent experiments for each cell line.

FIG. 18A-18C. depicts that intracellular SAM levels are not different between MTAP− and MTAP+ lines and do not correlate with MTA levels. a. Relative abundance of SAM from MTAP+(n=21) and MTAP− (n=19) cell lines from various lineages is shown. Mean of 3 biological replicates is displayed for each cell line. Median with upper and lower 25th percentiles is shown for MTAP+ and MTAP− lines. b. Normalized MTA and SAM levels are plotted for MTAP+ and MTAP− lines. Spearman rank correlation p-value is shown for MTAP− (red), MTAP+(blue), and all lines combined (black). c. Correlation of intracellular MTA/SAM molar ratios with sensitivity to PRMT5 depletion is shown. Results are similar to those shown in FIG. 3F (correlation of MTA with sensitivity to PRMT5 depletion). shPRMT5 sensitivity data from the screening and validation studies was normalized and combined using modified z-scores. Z-scores are plotted against intracellular MTA/SAM molar ratios for the 40 assayed cell lines. Spearman rank correlation p-value is shown.

FIG. 19. depicts a proposed mechanism by which MTAP loss leads to inhibition of PRMT5 activity and reduced cell viability in combination with genetic depletion of PRMT5.

### DETAILED DESCRIPTION OF THE INVENTION

The following detailed description is of example embodiments of the presently claimed invention with references to the accompanying drawings. Such description is intended to be illustrative and not limiting with respect to the scope of the present invention. Such embodiments are described in sufficient detail to enable one of ordinary skill in the art to practice the subject invention, and it will be understood that other embodiments may be practiced with some variations without departing from the spirit or scope of the subject invention.

The discovery of cancer dependencies has the potential to inform therapeutic strategies and to identify putative drug targets. Integrating data from comprehensive genomic profiling of cancer cell lines and from functional characterization of cancer cell dependencies, it was discovered that loss of the enzyme methylthioadenosine phosphorylase (MTAP) confers a selective dependence on protein arginine methyltransferase 5 (PRMT5) and its binding partner WDR77. MTAP is frequently lost due to its proximity to the commonly deleted tumor suppressor gene, CDKN2A. It was observed that there was increased intracellular concentrations of methylthioadenosine (MTA; the metabolite cleaved by MTAP) in cells harboring MTAP deletions. Furthermore, MTA specifically inhibited PRMT5 enzymatic activity. Administration of either MTA or a small molecule PRMT5 inhibitor showed a modest preferential impairment of cell viability for MTAP-null cancer cell lines compared to isogenic MTAP-expressing counterparts. The findings reveal PRMT5 as a vulnerability across multiple cancer lineages augmented by a common “passenger” genomic alteration. Thus, homozygous loss of MTAP in cancer cells confers a selective dependency on the protein arginine methyltransferase PRMT5.

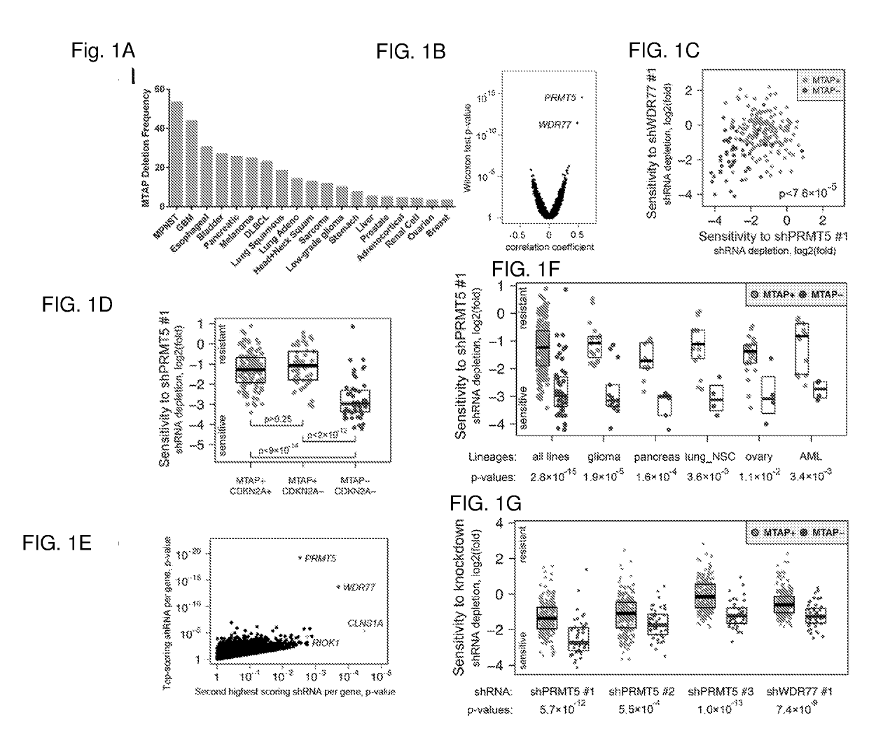

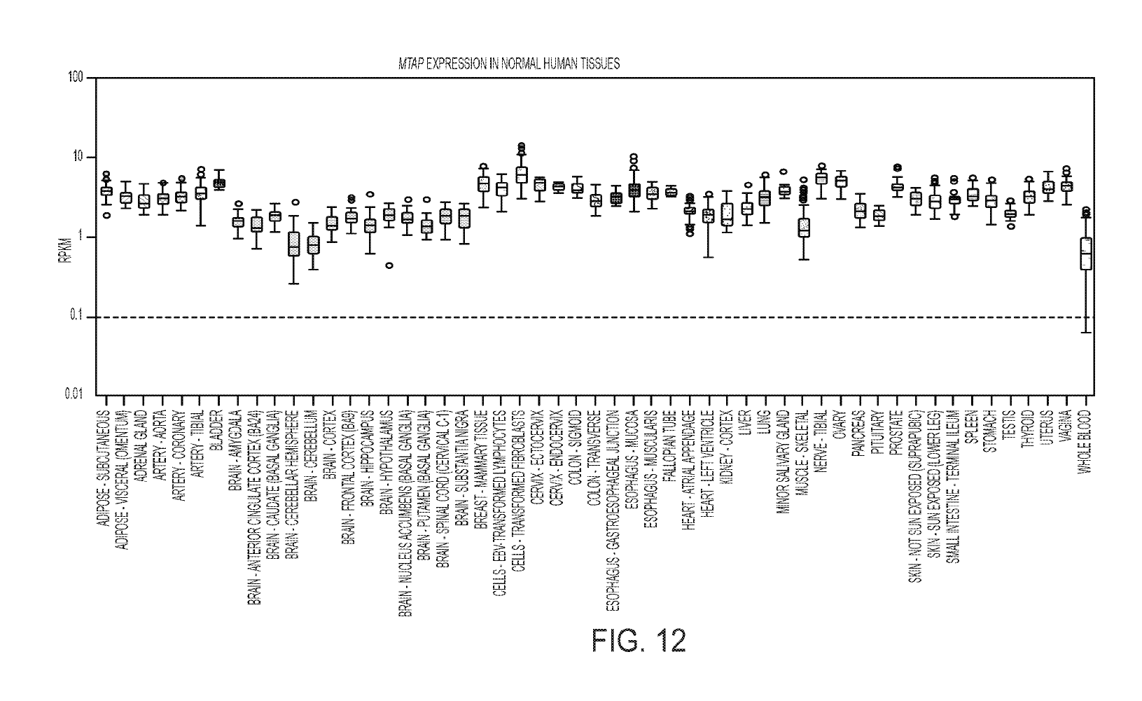

The gene encoding methylthioadenosine phosphorylase (MTAP) is ubiquitously expressed in normal tissues (FIG. 12). However, homozygous deletion of MTAP occurs frequently in cancer due to its proximity to CDKN2A, one of the most commonly deleted tumor suppressor genes (FIG. 1A) (1-7). For example, MTAP is deleted in 40% of glioblastomas; 25% of melanomas, urothelial carcinomas, and pancreatic adenocarcinomas; and 15% of non-small cell lung carcinomas (NSCLC) (8). MTAP cleaves methylthioadenosine (MTA) to generate precursor substrates for methionine and adenine salvage pathways. Synthetic lethal strategies to exploit MTAP loss with methionine starvation or by inhibiting de novo purine synthesis have been proposed; however, clinical efficacy of such approaches has not been demonstrated (9-11). It has been discovered that cancer cell lines with MTAP loss from multiple lineages show selective dependence on protein arginine methyltransferase 5 (PRMT5) and its binding partner WDR77 (also known as MEP50). As disclosed herein, MTAP loss produced increased intracellular MTA concentrations. In turn, elevated MTA inhibited PRMT5 specifically, thereby reducing PRMT5 enzymatic activity in cells harboring MTAP deletions. Further reduction of PRMT5 function by genetic knockdown impaired cell viability selectively in this context. Moreover, a small molecule PRMT5 inhibitor preferentially inhibited the viability of cancer cells harboring MTAP homozygous deletions. Together, these discoveries reveal a selective vulnerability across multiple cancer lineages conferred by a common “passenger” genomic alteration. In particular, PRMT5 represents a useful therapeutic target in the setting of MTAP loss, and pharmacologic inhibition of PRMT5 activity offers a new therapeutic avenue. Given the high frequency of MTAP deletion in cancer, this finding further provides new therapeutic methods for many patients with brain, lung, pancreatic, and other challenging cancers.

In one aspect, the invention provides a method of treating a tumor in a subject in which 5′-deoxy-5′-methylthioadenosine (MTA) levels are elevated, for example due to reduction of loss of methylthioadenosine phosphorylase (MTAP) activity, which comprises administering to the subject an effective amount of an inhibitor of protein arginine methyltransferase 5 (PRMT5). In another aspect, the invention provides a method of treating a tumor in a subject, which comprises administering an effective amount of an inhibitor of PRMT5 and an effective amount of an agent that elevates MTA levels. In some such embodiments, the agent that elevates MTA levels is an inhibitor of MTAP. In other such embodiments, the level of MTA is raised or supplemented by providing MTA to the tumor.

In another aspect, the invention provides a method of treating a tumor, which comprises administering an effective amount of an inhibitor of PRMT5 and an effective amount an MTA analog, or derivative. In one such embodiment, the MTA analog or derivative is an inhibitor of PRMT5, and is not a substrate of MTAP. In another embodiment, the MTA analog is both an inhibitor of PRMT5 and an inhibitor of MTAP.

In certain embodiments, an effective amount of a PRMT5 inhibitor is coadministered with one or more of MTA, an MTA analog or derivative, and an MTAP inhibitor.

In certain embodiments of the invention, the target cell is heterozygous or homozygous for an functional MTAP allele. MTAP inhibitors highly specific for each of these alleles or their RNA transcripts may be designed, for example, variance-specific antisense oligonucleotides, ribozymes, or small interfering RNAs (siRNAs). In certain embodiments, specific inhibitors (e.g., small molecules or peptides) of the variant MTAP-enzymes may be designed. In certain embodiments a tumor, in an MTAP-heterozygous patient, which has lost one of the alleles may be treated with a specific inhibitor of the remaining allele, or of its expressed MTAP-protein, to render the tumor effectively MTAP-negative.

The invention also features use of a CRISPR-based system engineered to inhibit MTAP expression. For example, according to the invention, a CRISPR-based system is engineered to express a hybrid CRISPR protein that comprises an effector domain that inhibits MTAP expression. In certain embodiments, the CRISPR protein is a CRISPR II family protein. In certain embodiments, the CRIRPR protein is Cas9. As detailed herein, the CRISPR protein can be, for example, Cas9 from Streptococcus thermophilus or Streptococcus pyogenes of other CRISPR protein such as are described herein.

Thus, the invention also features the use of small nucleic acid molecules, referred to as short interfering nucleic acid (siNA) that include, for example: microRNA (miRNA), short interfering RNA (siRNA), double-stranded RNA (dsRNA), and short hairpin RNA (shRNA) molecules to knockdown expression of proteins such as MTAP. An siNA of the invention can be unmodified or chemically-modified. An siNA of the instant invention can be chemically synthesized, expressed from a vector or enzymatically synthesized. The instant invention also features various chemically-modified synthetic short interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic applications.

When a PRMT5 inhibitor is coadministered with a second agent, such as MTA, or an MTA analog, or an agent that inhibits MTAP, the PRMT5 inhibitor and the second agent may be administered together or separately, at the same or different times and by the same or different route of administration.

According to the invention, the amount or concentration of PRMT5 inhibitor used to obtain a predetermined or desired result is reduced in cells or tissues that have reduced or substantially no MTAP activity or increased inhibition of PRMT5 by MTA.

According to the invention, the PRMT5 inhibitor can be a conjugate, for example, a PRMT5 inhibitor linked to MTA, or an MTA analog, or an MTAP inhibitor. The linker can be hydrolysable or stable.

The term “combination” embraces the administration of a PRMT inhibitor and MTA, or an MTA analog, or an agent that inhibits MTAP. The combination may also include one or more additional agents, for example, but not limited to, chemotherapeutic agents, anti-angiogenesis agents and agents that reduce immune-suppression. The beneficial effect of the combination includes, but is not limited to, pharmacokinetic or pharmacodynamic co-action resulting from the combination of therapeutic agents. Administration of these therapeutic agents in combination typically is carried out over a defined time period (for example, minutes, hours, days, or weeks depending upon the combination selected).

“Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of tumor specific neoantigens and a checkpoint inhibitor administered at the same or different times, or they can be formulated as a single, co-formulated pharmaceutical composition comprising the two compounds. As another example, a combination of the present invention (e.g., a pooled sample of tumor specific neoantigens and a checkpoint inhibitor and/or an anti-CTLA4 antibody) may be formulated as separate pharmaceutical compositions that can be administered at the same or different time. As used herein, the term “simultaneously” is meant to refer to administration of one or more agents at the same time. For example, in certain embodiments, a neoplasia vaccine or immunogenic composition and a checkpoint inhibitor are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neoantigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

By a “polypeptide” or “peptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “receptor” is to be understood as meaning a biological molecule or a molecule grouping capable of binding a ligand. A receptor may serve, to transmit information in a cell, a cell formation or an organism. The receptor comprises at least one receptor unit and frequently contains two or more receptor units, where each receptor unit may consist of a protein molecule, in particular a glycoprotein molecule. The receptor has a structure that complements the structure of a ligand and may complex the ligand as a binding partner. Signaling information may be transmitted by conformational changes of the receptor following binding with the ligand on the surface of a cell. According to the invention, a receptor may refer to particular proteins of MHC classes I and II capable of forming a receptor/ligand complex with a ligand, in particular a peptide or peptide fragment of suitable length.

The term “subject” refers to an animal that is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the combination therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., EDO of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The term “ablate” refers to removal or destruction of a cell. Regarding MTAP negative tumor cells or cells with decreased MTAP expression or activity, ablation refers to the removal or destruction of these cells. In preferred embodiments, removal or destruction by inhibition of PRMT5 is caused by growth inhibition, apoptosis, or a combination of both.

The terms “effective amount” or “amount effective to” or “therapeutically effective amount” means an amount of an agent sufficient to produce a desired result, for example, killing a cancer cell, reducing tumor cell proliferation, reducing inflammation in a diseased tissue or organ, or labeling a specific population of cells in a tissue, organ, or organism (e.g., a human).

The term “linker” is refers to a covalent tether or connector which is joins an MTA, an MTA analog, or an MTAP inhibitor (such as compounds of Formula I) with binding moieties, diagnostic agents, or therapeutic agents.

The term “connector” is meant an amino acid sequence of 2 to 20 residues in length that is covalently attached to one or more residues of an MTA, an MTA analog, or an MTAP inhibitor.

“Treating” preferably provides a reduction (e.g., by at least 10%, 15%, 20%, 25%, 30%, 35%, 40%, 45%, 50%, 60%, 70%, 80%, 90%, 95%, 99%, or even 100%) in the progression or severity of a human disease or disorder (e.g., an autoimmune or proliferative disease), or in the progression, severity, or frequency of one or more symptoms of the human disease or disorder in a subject.

The term “derivative” refers to polypeptides derived from naturally occurring compounds by chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

Similarly, the term “analog” refers to molecular compounds that differ in at least one or more atoms, functional groups, or substructures, which are replaced with other atoms, groups, or substructures.

The PRMT5 inhibitor may be any compound or molecule that leads to a decrease in enzymatic activity. The enzymatic activity is protein arginine methyltransferase activity. The inhibitor may be YQ36286 as described in a presentation entitled “Identification of a First-in-Class PRMT5 Inhibitor with Potent in Vitro and in Vivo Activity in Preclinical Models of Mantle Cell Lymphoma” and can be found at ash.confex.com/ash/2014/webprogram/Paper70118.html. Additionally, the PRMT5 inhibitor, EPZ015666, has been described in the Jan. 15, 2015 issue of Biocentury Innovations. Additional PRMT5 inhibitors have also been described (www.cancertechnology.co.uk/inhibitors-protein-arginine-methyltransferase-5-prmt5). PRMT5 inhibitors may also be any of the compounds described in U.S. Pat. Nos. 8,940,726 and 8,906,900, and U.S. patent application numbers 20140329794, 20140228360, 20140228343, 20140221345 and 20140213582, herein incorporated by reference in their entirety.

MTAP inhibitors have been described. The inhibitor may be MT-DADMe-Immucillin A (Chattopadhyay et al., 2006, Mol Cancer Ther, 5:2549). Other MTAP inhibitors include DADMe-Immucillin H, Immucillin H and DADMe-Immucillin G.

The present invention also contemplates the use of nucleic acid molecules as vehicles for delivering inhibitors of MTAP, PRMT5, or any additional drug to be administered as a combination therapy to the subject in need thereof, in vivo, in the form of, e.g., DNA/RNA vectors (see, e.g., WO2012/159643, and WO2012/159754, hereby incorporated by reference in their entirety). For example, an inhibitor may be a microRNA, shRNA, CRISPR-Cas system that targets MTAP.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, and 8,795,965; allowed U.S. application Ser. No. 14/259,420; US Patent Publications US 2014-0179006 A1 (U.S. application Ser. No. 14/183,486), US 2014-0189896 A1 (U.S. application Ser. No. 14/105,035), US 2014-0186843 A1 (U.S. application Ser. No. 14/104,900), US 2014-0179770 A1 (U.S. application Ser. No. 14/104,837), US 2014-0186919 A1 (U.S. application Ser. No. 14/104,977), and US 2014-186958 A1 (U.S. application Ser. No. 14/105,017): PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), and WO2014/018423 (PCT/US2013/051418); U.S. provisional applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent applications 61/836,123, 61/847,537, 61/862,355 and 61/871,301, filed on Jun. 17, 2013; Jul. 17, 2013, Aug. 5, 2013 and Aug. 28, 2013 respectively. Reference is further made to U.S. provisional patent applications 61/736,527 and 61/748,427 filed on Dec. 12, 2012 and Jan. 2, 2013, respectively. Reference is additionally made to U.S. provisional patent application 61/791,409, filed on Mar. 15, 2013. Reference is also made to U.S. provisional patent application 61/799,800, filed Mar. 15, 2013. Reference is also made to U.S. provisional patent applications 61/835,931, 61/835,936, 61/836,127, 61/836,101, 61/836,123, 61/836,080, and 61/835,973 each filed Jun. 17, 2013. Reference is made to U.S. provisional patent application 61/915,118, filed on Dec. 12, 2013 and U.S. provisional patent application 62/010,441 filed Jun. 10, 2014, each of which is incorporated herein by reference. Reference is also made to U.S. provisional patent applications 61/915,215 and 61/915,148, both filed on Dec. 12, 2013, each of which is incorporated herein by reference. Each of these applications, and all documents cited therein or during their prosecution (“appin cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these applications and the appin cited documents) are incorporated herein by reference to the same extent as if each individual document was specifically and individually indicated to be incorporated by reference. Citations for documents cited herein may also be found in the foregoing herein-cited documents, as well as those hereinbelow cited.

Also with respect to general information on CRISPR-Cas Systems, mention is made of:
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23;
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5. (2013);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308. (2013);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156(5):935-49;
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889,
Development and Applications of CRISPR-Cas9 for Genome Engineering, Hsu et al, Cell 157, 1262-1278 (Jun. 5, 2014) (Hsu 2014), and
CRISPR: new horizons in phage resistance and strain identification. Barrangou, R; Horvath P. Annu Rev Food Sci Technol. (2012), 3:143-162. doi: 10.1146/annurev-food-022811-101134.
   each of which is incorporated herein by reference, and discussed briefly below:

Cong et al. engineered type II CRISPR/Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR/Cas system can be further improved to increase its efficiency and versatility.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

Konermann et al. addressed the need in the art for versatile and robust technologies that enable optical and chemical modulation of DNA-binding domains based CRISPR Cas9 enzyme and also Transcriptional Activator Like Effectors

Cas9 nuclease from the microbial CRISPR-Cas system is targeted to specific genomic loci by a 20 nt guide sequence, which can tolerate certain mismatches to the DNA target and thereby promote undesired off-target mutagenesis. To address this, Ran et al. described an approach that combined a Cas9 nickase mutant with paired guide RNAs to introduce targeted double-strand breaks. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.

Hsu et al. characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors reported that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.

Ran et al. described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors provides experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.

Nishimasu et al. reported the crystal structure of Streptococcus pyogenes Cas9 in complex with sgRNA and its target DNA at 2.5 A° resolution. The structure revealed a bi-lobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.

Hsu 2014 is a review article that discusses generally CRISPR-Cas9 history from yogurt to genome editing, including genetic screening of cells, that is in the information, data and findings of the applications in the lineage of this specification filed prior to Jun. 5, 2014. The general teachings of Hsu 2014 do not involve the specific models, animals of the instant specification.

Barrangou et al. found that the CRISPR system immunize microorganisms against phages and plasmids while simultaneously directing their evolution. The authors generally discuss the background of the use of bacterial, widely known to be used as starter culture in the food industry, specifically and most notably for the fermentation of milk into dairy products such as cheese and yogurt. The authors state that given viral exposure in industrial environments, starter culture selection and development rely on defense systems that provide resistance against bacteriophage predation, including restriction-modification, abortive infection, and recently discovered CRISPR systems. Thus, the authors propose utilization of CRISPR transcripts that are processed into small interfering RNAs that guide a multifunctional protein complex to recognize and cleave matching foreign DNA.

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree secondary structure within the guide sequence. In some embodiments, about or less than about 75%, 50%, 40%, 30%, 25%, 20%, 15%, 10%, 5%, 1%, or fewer of the nucleotides of the guide sequence participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62).

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNNNNNNNNNNNNNgtttttgtactctcaagatttaGAAAtaaatcttgcagaagctacaaagataa ggatcatgccgaaatcaacaccctgtcattttatggcagggtgttttcgttatttaaTTTTTT (SEQ ID NO: 3); (2) NNNNNNNNNNNNNNNNNNNNgtttttgtactctcaGAAAtgcagaagctacaaagataaggcttcatgccg aaatcaacaccctgtcattttatggcagggtgttttcgttatttaaTTTTTT (SEQ ID NO: 4); (3) NNNNNNNNNNNNNNNNNNNNgtttttgtactctcaGAAAtgcagaagctacaaagataaggcttcatgccg aaatcaacaccctgtcattttatggcagggtgtTTTTTT (SEQ ID NO: 5); (4) NNNNNNNNNNNNNNNNNNNNgttttagagctaGAAAtagcaagttaaaataaggctagtccgttatcaactt gaaaaagtggcaccgagtcggtgcTTTTTT (SEQ ID NO: 6); (5) NNNNNNNNNNNNNNNNNNNNgttttagagctaGAAATAGcaagttaaaataaggctagtccgttatcaac ttgaaaaagtgTTTTTTT (SEQ ID NO: 7); and (6) NNNNNNNNNNNNNNNNNNNNgttttagagctagAAATAGcaagttaaaataaggctagtccgttatcaTT TTTTTT (SEQ ID NO: 8). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, candidate tracrRNA may be subsequently predicted by sequences that fulfill any or all of the following criteria: 1. sequence homology to direct repeats (motif search in Geneious with up to 18-bp mismatches); 2. presence of a predicted Rho-independent transcriptional terminator in direction of transcription; and 3. stable hairpin secondary structure between tracrRNA and direct repeat. In some embodiments, 2 of these criteria may be used, for instance 1 and 2, 2 and 3, or 1 and 3. In some embodiments, all 3 criteria may be used.

In some embodiments, chimeric synthetic guide RNAs (sgRNAs) designs may incorporate at least 12 bp of duplex structure between the direct repeat and tracrRNA.

For minimization of toxicity and off-target effect, it will be important to control the concentration of CRISPR enzyme mRNA and guide RNA delivered. Optimal concentrations of CRISPR enzyme mRNA and guide RNA can be determined by testing different concentrations in a cellular or non-human eukaryote animal model and using deep sequencing the analyze the extent of modification at potential off-target genomic loci. For example, for the guide sequence targeting 5′-GAGTCCGAGCAGAAGAAGAA-3′ (SEQ ID NO: 9) in the EMX1 gene of the human genome, deep sequencing can be used to assess the level of modification at the following two off-target loci, 1: 5′-GAGTCCTAGCAGGAGAAGAA-3′ (SEQ ID NO: 10) and 2: 5′-GAGTCTAAGCAGAAGAAGAA-3′ (SEQ ID NO: 11). The concentration that gives the highest level of on-target modification while minimizing the level of off-target modification should be chosen for in vivo delivery. Alternatively, to minimize the level of toxicity and off-target effect, CRISPR enzyme nickase mRNA (for example S. pyogenes Cas9 with the D10A mutation) can be delivered with a pair of guide RNAs targeting a site of interest. The two guide RNAs need to be spaced as follows. Guide sequences and strategies to minimize toxicity and off-target effects can be as in WO 2014/093622 (PCT/US2013/074667).

The CRISPR system is derived advantageously from a type II CRISPR system. In some embodiments, one or more elements of a CRISPR system is derived from a particular organism comprising an endogenous CRISPR system, such as Streptococcus pyogenes. In preferred embodiments of the invention, the CRISPR system is a type II CRISPR system and the Cas enzyme is Cas9, which catalyzes DNA cleavage. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof.

In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. Where the enzyme is not SpCas9, mutations may be made at any or all residues corresponding to positions 10, 762, 840, 854, 863 and/or 986 of SpCas9 (which may be ascertained for instance by standard sequence comparison tools). In particular, any or all of the following mutations are preferred in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s are also preferred. Particularly preferred are D10 and H840 in SpCas9. However, in other Cas9s, residues corresponding to SpCas9 D10 and H840 are also preferred. Orthologs of SpCas9 can be used in the practice of the invention. A Cas enzyme may be identified Cas9 as this can refer to the general class of enzymes that share homology to the biggest nuclease with multiple nuclease domains from the type II CRISPR system. Most preferably, the Cas9 enzyme is from, or is derived from, spCas9 (S. pyogenes Cas9) or saCas9 (S. aureus Cas9). StCas9″ refers to wild type Cas9 from S. thermophilus, the protein sequence of which is given in the SwissProt database under accession number G3ECR1. Similarly, S pyogenes Cas9 or spCas9 is included in SwissProt under accession number Q99ZW2. By derived, Applicants mean that the derived enzyme is largely based, in the sense of having a high degree of sequence homology with, a wildtype enzyme, but that it has been mutated (modified) in some way as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this invention includes many more Cas9s from other species of microbes, such as SpCas9, SaCa9, St1Cas9 and so forth. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defence in bacteria and archaea, Mole Cell 2010, Jan. 15; 37(1): 7. The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions, one can generate chimeric Cas9 proteins. And Cas9 may be used as a generic DNA binding protein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme comprising one or more nuclear localization sequences (NLSs), such as about or more than about 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 12); the NLS from nucleoplasmin (e.g. the nucleoplasmin bipartite NLS with the sequence KRPAATKKAGQAKKKK (SEQ ID NO: 13)); the c-myc NLS having the amino acid sequence PAAKRVKLD (SEQ ID NO: 14) or RQRRNELKRSP (SEQ ID NO: 15); the hRNPA1 M9 NLS having the sequence NQSSNFGPMKGGNFGGRSSGPYGGGGQYFAKPRNQGGY (SEQ ID NO: 16); the sequence RMRIZFKNKGKDTAELRRRRVEVSVELRKAKKDEQILKRRNV (SEQ ID NO: 17) of the IBB domain from importin-alpha; the sequences VSRKRPRP (SEQ ID NO: 18) and PPKKARED (SEQ ID NO: 19) of the myoma T protein; the sequence PQPKKKPL (SEQ ID NO: 20) of human p53; the sequence SALIKKKKKMAP (SEQ ID NO: 21) of mouse c-abl IV; the sequences DRLRR (SEQ ID NO: 22) and PKQKKRK (SEQ ID NO: 23) of the influenza virus NS1; the sequence RKLKKKIKKL (SEQ ID NO: 24) of the Hepatitis virus delta antigen; the sequence REKKKFLKRR (SEQ ID NO: 25) of the mouse Mx1 protein; the sequence KRKGDEVDGVDEVAKKKSKK (SEQ ID NO: 26) of the human poly(ADP-ribose) polymerase; and the sequence RKCLQAGMNLEARKTKK (SEQ ID NO: 27) of the steroid hormone receptors (human) glucocorticoid. In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

Aspects of the invention relate to the expression of the gene product being decreased or a template polynucleotide being further introduced into the DNA molecule encoding the gene product or an intervening sequence being excised precisely by allowing the two 5′ overhangs to reanneal and ligate or the activity or function of the gene product being altered or the expression of the gene product being increased. In an embodiment of the invention, the gene product is a protein. Only sgRNA pairs creating 5′ overhangs with less than 8 bp overlap between the guide sequences (offset greater than −8 bp) were able to mediate detectable indel formation. Importantly, each guide used in these assays is able to efficiently induce indels when paired with wildtype Cas9, indicating that the relative positions of the guide pairs are the most important parameters in predicting double nicking activity. Since Cas9n and Cas9H840A nick opposite strands of DNA, substitution of Cas9n with Cas9H840A with a given sgRNA pair should have resulted in the inversion of the overhang type; but no indel formation is observed as with Cas9H840A indicating that Cas9H840A is a CRISPR enzyme substantially lacking all DNA cleavage activity (which is when the DNA cleavage activity of the mutated enzyme is about no more than 25%, 10%, 5%, 1%, 0.1%, 0.01%, or less of the DNA cleavage activity of the non-mutated form of the enzyme; whereby an example can be when the DNA cleavage activity of the mutated form is nil or negligible as compared with the non-mutated form, e.g., when no indel formation is observed as with Cas9H840A in the eukaryotic system in contrast to the biochemical or prokaryotic systems). Nonetheless, a pair of sgRNAs that will generate a 5′ overhang with Cas9n should in principle generate the corresponding 3′ overhang instead, and double nicking. Therefore, sgRNA pairs that lead to the generation of a 3′ overhang with Cas9n can be used with another mutated Cas9 to generate a 5′ overhang, and double nicking. Accordingly, in some embodiments, a recombination template is also provided. A recombination template may be a component of another vector as described herein, contained in a separate vector, or provided as a separate polynucleotide. In some embodiments, a recombination template is designed to serve as a template in homologous recombination, such as within or near a target sequence nicked or cleaved by a CRISPR enzyme as a part of a CRISPR complex. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Or, RNA(s) of the CRISPR System can be delivered to a transgenic Cas9 animal or mammal, e.g., an animal or mammal that constitutively or inducibly or conditionally expresses Cas9. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter. Delivery vehicles, vectors, particles, nanoparticles, formulations and components thereof for expression of one or more elements of a CRISPR system are as used in the foregoing documents, such as WO 2014/093622 (PCT/US2013/074667). In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell. In some embodiments, a vector comprises a regulatory element operably linked to an enzyme-coding sequence encoding a CRISPR enzyme, such as a Cas protein. CRISPR enzyme or CRISPR enzyme mRNA or CRISPR guide RNA or RNA(s) can be delivered separately; and advantageously at least one of these is delivered via a nanoparticle complex. CRISPR enzyme mRNA can be delivered prior to the guide RNA to give time for CRISPR enzyme to be expressed. CRISPR enzyme mRNA might be administered 1-12 hours (preferably around 2-6 hours) prior to the administration of guide RNA. Alternatively, CRISPR enzyme mRNA and guide RNA can be administered together. Advantageously, a second booster dose of guide RNA can be administered 1-12 hours (preferably around 2-6 hours) after the initial administration of CRISPR enzyme mRNA+ guide RNA. Additional administrations of CRISPR enzyme mRNA and/or guide RNA might be useful to achieve the most efficient levels of genome modification.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types. As such the CRISPR complex of the invention has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide. The guide sequence is linked to a tracr mate sequence, which in turn hybridizes to a tracr sequence. In one embodiment, this invention provides a method of cleaving a target polynucleotide. The method comprises modifying a target polynucleotide using a CRISPR complex that binds to the target polynucleotide and effect cleavage of said target polynucleotide. Typically, the CRISPR complex of the invention, when introduced into a cell, creates a break (e.g., a single or a double strand break) in the genome sequence. For example, the method can be used to cleave a disease gene in a cell. The break created by the CRISPR complex can be repaired by a repair processes such as the error prone non-homologous end joining (NHEJ) pathway or the high fidelity homology-directed repair (HDR). During these repair process, an exogenous polynucleotide template can be introduced into the genome sequence. In some methods, the HDR process is used modify genome sequence. For example, an exogenous polynucleotide template comprising a sequence to be integrated flanked by an upstream sequence and a downstream sequence is introduced into a cell. The upstream and downstream sequences share sequence similarity with either side of the site of integration in the chromosome. Where desired, a donor polynucleotide can be DNA, e.g., a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function. The upstream and downstream sequences in the exogenous polynucleotide template are selected to promote recombination between the chromosomal sequence of interest and the donor polynucleotide. The upstream sequence is a nucleic acid sequence that shares sequence similarity with the genome sequence upstream of the targeted site for integration. Similarly, the downstream sequence is a nucleic acid sequence that shares sequence similarity with the chromosomal sequence downstream of the targeted site of integration. The upstream and downstream sequences in the exogenous polynucleotide template can have 75%, 80%, 85%, 90%, 95%, or 100% sequence identity with the targeted genome sequence. Preferably, the upstream and downstream sequences in the exogenous polynucleotide template have about 95%, 96%, 97%, 98%, 99%, or 100% sequence identity with the targeted genome sequence. In some methods, the upstream and downstream sequences in the exogenous polynucleotide template have about 99% or 100% sequence identity with the targeted genome sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the invention can be constructed using recombinant techniques (see, for example, Sambrook et al., 2001 and Ausubel et al., 1996). In a method for modifying a target polynucleotide by integrating an exogenous polynucleotide template, a double stranded break is introduced into the genome sequence by the CRISPR complex, the break is repaired via homologous recombination an exogenous polynucleotide template such that the template is integrated into the genome. The presence of a double-stranded break facilitates integration of the template. In other embodiments, this invention provides a method of modifying expression of a polynucleotide in a eukaryotic cell. The method comprises increasing or decreasing expression of a target polynucleotide by using a CRISPR complex that binds to the polynucleotide. In some methods, a target polynucleotide can be inactivated to effect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein or microRNA or pre-microRNA transcript is not produced. In some methods, a control sequence can be inactivated such that it no longer functions as a control sequence. As used herein, “control sequence” refers to any nucleic acid sequence that effects the transcription, translation, or accessibility of a nucleic acid sequence. Examples of a control sequence include, a promoter, a transcription terminator, and an enhancer are control sequences. The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence) Examples of PAM sequences are given in the examples section below, and the skilled person will be able to identify further PAM sequences for use with a given CRISPR enzyme. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. In one aspect, the invention provides a method of modifying expression of a polynucleotide in a eukaryotic cell. In some embodiments, the method comprises allowing a CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence. Similar considerations and conditions apply as above for methods of modifying a target polynucleotide. In fact, these sampling, culturing and re-introduction options apply across the aspects of the present invention. In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may even be re-introduced into the non-human animal or plant. For re-introduced cells it is particularly preferred that the cells are stem cells.

Indeed, in any aspect of the invention, the CRISPR complex may comprise a CRISPR enzyme complexed with a guide sequence hybridized to a target sequence, wherein said guide sequence may be linked to a tracr mate sequence which in turn may hybridize to a tracr sequence.

In one embodiment inhibitors may be administered to a patient in need thereof by use of a plasmid. These are plasmids which usually consist of a strong viral promoter to drive the in vivo transcription and translation of the gene (or complementary DNA) of interest (Mor, et al., (1995). The Journal of Immunology 155 (4): 2039-2046). Intron A may sometimes be included to improve mRNA stability and hence increase protein expression (Leitner et al. (1997). The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999). Adv. Parasitol. Advances in Parasitology 42: 343-410; Robinson et al., (2000). Adv. Virus Res. Advances in Virus Research 55: 1-74; Böhm et al., (1996). Journal of Immunological Methods 193 (1): 29-40.). Multicistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimizing vector design for maximal protein expression is essential (Lewis et al., (1999). Advances in Virus Research (Academic Press) 54: 129-88). One way of enhancing protein expression is by optimizing the codon usage of pathogenic mRNAs for eukaryotic cells. Another consideration is the choice of promoter. Such promoters may be the SV40 promoter or Rous Sarcoma Virus (RSV).

Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors.

The method of delivery determines the dose of DNA required. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshia et al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

One or more inhibitors of the invention may be encoded and expressed in vivo using a viral based system (e.g., an adenovirus system, an adeno associated virus (AAV) vector, a poxvirus, or a lentivirus). In one embodiment, the inhibitor may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

The inhibitors of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the inhibitor of the invention.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571; US20040013648, US20070025970, US20090111106 and U.S. Pat. No. 7,259,015. In another embodiment lentiviral vectors are used to deliver vectors to the brain of those being treated for a disease.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

In an embodiment herein the delivery is via a lentivirus. Zou et al. administered about 10 μl of a recombinant lentivirus having a titer of 1×10⁹ transducing units (TU)/ml by an intrathecal catheter. These sorts of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹ TU/ml, more preferably at least 10⁹ TU/ml. Other methods of concentration such as ultrafiltration or binding to and elution from a matrix may be used.

In other embodiments the amount of lentivirus administered may be 1.×.10⁵ or about 1.×.10⁵ plaque forming units (PFU), 5.×.10⁵ or about 5.×.10⁵ PFU, 1.×.10⁶ or about 1.×10⁶ PFU, 5.×.10⁶ or about 5.×.10⁶ PFU, 1.×.10⁷ or about 1.×.10⁷ PFU, 5.×.10⁷ or about 5.×.10⁷ PFU, 1.×.10⁸ or about 1.×.10⁸ PFU, 5.×.10⁸ or about 5.×.10⁸ PFU, 1.×.10⁹ or about 1.×.10⁹ PFU, 5.×.10⁹ or about 5.×.10⁹ PFU, 1.×.10¹⁰ or about 1.×.10¹⁰ PFU or 5.×.10¹⁰ or about 5.×.10¹⁰ PFU as total single dosage for an average human of 75 kg or adjusted for the weight and size and species of the subject. One of skill in the art can determine suitable dosage. Suitable dosages for a virus can be determined empirically.

Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MEW class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both E1a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10″ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10″ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10″ pu, about 2×10″ pu, about 4×10″ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA).

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment a Poxvirus is used to express an inhibitor. These include orthopoxvirus, avipox, vaccinia, MVA, NYVAC, canarypox, ALVAC, fowlpox, TROVAC, etc. (see e.g., Verardi et al., Hum Vaccin Immunother. 2012 July; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, W R Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature, such as:
U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and
Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith G L. Proc. Natl. Acad. Sci. 1983; 80(23): 7155-9, Smith G L. Nature 1983; 302: 490-5, Sullivan V J. Gen. Vir. 1987; 68: 2587-98, Perkus M Journal of Leukocyte Biology 1995; 58:1-13, Yilma T D. Vaccine 1989; 7: 484-485, Brochier B. Nature 1991; 354: 520-22, Wiktor, T J. Proc. Natl Acd. Sci. 1984; 81: 7194-8, Rupprecht, C E. Proc. Natl Acd. Sci. 1986; 83: 7947-50, Poulet, H Vaccine 2007; 25(July): 5606-12, Weyer J. Vaccine 2009; 27(November): 7198-201, Buller, R M Nature 1985; 317(6040): 813-5, Buller R M. J. Virol. 1988; 62(3):866-74, Flexner, C. Nature 1987; 330(6145): 259-62, Shida, H. J. Virol. 1988; 62(12): 4474-80, Kotwal, G J. J. Virol. 1989; 63(2): 600-6, Child, S J. Virology 1990; 174(2): 625-9, Mayr A. Zentralbl Bakteriol 1978; 167(5,6): 375-9, Antoine G. Virology. 1998; 244(2): 365-96, Wyatt, L S. Virology 1998; 251(2): 334-42, Sancho, M C. J. Virol. 2002; 76(16); 8313-34, Gallego-Gomez, J C. J. Virol. 2003; 77(19); 10606-22), Goebel S J. Virology 1990; (a,b) 179: 247-66, Tartaglia, J. Virol. 1992; 188(1): 217-32, Najera J L. J. Virol. 2006; 80(12): 6033-47, Najera, J L. J. Virol. 2006; 80: 6033-6047, Gomez, C E. J. Gen. Virol. 2007; 88: 2473-78, Mooij, P. Jour. Of Virol. 2008; 82: 2975-2988, Gomez, C E. Curr. Gene Ther. 2011; 11: 189-217, Cox, W. Virology 1993; 195: 845-50, Perkus, M. Jour. Of Leukocyte Biology 1995; 58: 1-13, Blanchard T J. J Gen Virology 1998; 79(5): 1159-67, Amara R. Science 2001; 292: 69-74, Hel, Z., J. Immunol. 2001; 167: 7180-9, Gherardi M M. J. Virol. 2003; 77: 7048-57, Didierlaurent, A. Vaccine 2004; 22: 3395-3403, Bissht H. Proc. Nat. Aca. Sci. 2004; 101: 6641-46, McCurdy L H. Clin. Inf. Dis 2004; 38: 1749-53, Earl P L. Nature 2004; 428: 182-85, Chen Z. J. Virol. 2005; 79: 2678-2688, Najera J L. J. Virol. 2006; 80(12): 6033-47, Nam J H. Acta. Virol. 2007; 51: 125-30, Antonis A F. Vaccine 2007; 25: 4818-4827, B Weyer J. Vaccine 2007; 25: 4213-22, Ferrier-Rembert A. Vaccine 2008; 26(14): 1794-804, Corbett M. Proc. Natl. Acad. Sci. 2008; 105(6): 2046-51, Kaufman H L., J. Clin. Oncol. 2004; 22: 2122-32, Amato, R J. Clin. Cancer Res. 2008; 14(22): 7504-10, Dreicer R. Invest New Drugs 2009; 27(4): 379-86, Kantoff P W. J. Clin. Oncol. 2010, 28, 1099-1105, Amato R J. J. Clin. Can. Res. 2010; 16(22): 5539-47, Kim, D W. Hum. Vaccine. 2010; 6: 784-791, Oudard, S. Cancer Immunol. Immunother. 2011; 60: 261-71, Wyatt, L S. Aids Res. Hum. Retroviruses. 2004; 20: 645-53, Gomez, C E. Virus Research 2004; 105: 11-22, Webster, D P. Proc. Natl. Acad. Sci. 2005; 102: 4836-4, Huang, X. Vaccine 2007; 25: 8874-84, Gomez, C E. Vaccine 2007a; 25: 2863-85, Esteban M. Hum. Vaccine 2009; 5: 867-871, Gomez, C E. Curr. Gene therapy 2008; 8(2): 97-120, Whelan, K T. Plos one 2009; 4(6): 5934, Scriba, T J. Eur. Jour. Immuno. 2010; 40(1): 279-90, Corbett, M. Proc. Natl. Acad. Sci. 2008; 105: 2046-2051, Midgley, C M. J. Gen. Virol. 2008; 89: 2992-97, Von Krempelhuber, A. Vaccine 2010; 28: 1209-16, Perreau, M. J. Of Virol. 2011; October: 9854-62, Pantaleo, G. Curr Opin HIV-AIDS. 2010; 5: 391-396,
   each of which is incorporated herein by reference.

In another embodiment the vaccinia virus is used as a vector. (Rolph et al., Recombinant viruses as vaccines and immunological tools. Curr Opin Immunol 9:517-524, 1997).

In another embodiment ALVAC is used as a vector. ALVAC is a canarypox virus that can be modified to express foreign transgenes and has been used as a method for vaccination against both prokaryotic and eukaryotic antigens (Honig H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6:2219-28; Musey L, Ding Y, Elizaga M, et al. HIV-1 vaccination administered intramuscularly can induce both systemic and mucosal T cell immunity in HIV-1-uninfected individuals. J Immunol 2003; 171:1094-101; Paoletti E. Applications of pox virus vectors to vaccination: an update. Proc Natl Acad Sci USA 1996; 93:11349-53; U.S. Pat. No. 7,255,862).

In another embodiment a Modified Vaccinia Ankara (MVA) virus may be used as a viral vector. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61(1): 19-29). Methods to make and use recombinant MVA has been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles are administered to patients in need thereof. The viral particles can be administered to a patient in need thereof or transfected into cells in an amount of about at least 10^{3.5} pfu; thus, the viral particles are preferably administered to a patient in need thereof or infected or transfected into cells in at least about 10⁴ pfu to about 10⁶ pfu; however, a patient in need thereof can be administered at least about 10⁸ pfu such that a more preferred amount for administration can be at least about 10⁷ pfu to about 10⁹ pfu. Doses as to NYVAC are applicable as to ALVAC, MVA, MVA-BN, and avipoxes, such as canarypox and fowlpox.

Examples of cancers and cancer conditions that can be treated with the combination therapy of this document include, but are not limited to a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites.

Of special interest is the treatment of Non-Hodgkin's Lymphoma (NHL), clear cell Renal Cell Carcinoma (ccRCC), metastatic melanoma, sarcoma, leukemia or a cancer of the bladder, colon, brain, breast, head and neck, endometrium, lung, ovary, pancreas or prostate. In certain embodiments, the melanoma is high risk melanoma.

Cancers that can be treated using this combination therapy may include among others cases that are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The combination therapy described herein is also applicable to the treatment of patients in need thereof who have not been previously treated.

The combination therapy described herein is also applicable where the subject has no detectable neoplasia but is at high risk for disease recurrence.

The present invention is also directed to pharmaceutical compositions comprising an effective amount of one or more compounds according to the present invention (including a pharmaceutically acceptable salt, thereof), optionally in combination with a pharmaceutically acceptable carrier, excipient or additive.

When administered as a combination, the therapeutic agents can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a neoplasia.

In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

The pharmaceutical compositions can be processed in accordance with conventional methods of pharmacy to produce medicinal agents for administration to patients in need thereof, including humans and other mammals.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as a fatty oil. In addition, dosage unit forms can contain various other materials that modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose.

In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 1-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns that is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier usually comprises sterile water or aqueous sodium chloride solution, though other ingredients including those that aid dispersion may be included. Of course, where sterile water is to be used and maintained as sterile, the compositions and carriers are also sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

The inhibitor, and any additional agents, may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

In certain embodiments, the inhibitors are administered intravenously or subcutaneously.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The inhibitors may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The inhibitors may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxyuridine, ara-C, hydroxyurea and thioguanine; antibodies such as HERCEPTIN and RITUXAN.

It should be understood that in addition to the ingredients particularly mentioned herein, the formulations of the present invention may include other agents conventional in the art having regard to the type of formulation in question, for example, those suitable for oral administration may include flavoring agents.

Pharmaceutically acceptable salt forms may be the preferred chemical form of compounds according to the present invention for inclusion in pharmaceutical compositions according to the present invention.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein is evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

### Dosage

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with viral infection and/or autoimmune disease.

A preferred dose of an agent is the maximum that a patient can tolerate and not develop serious or unacceptable side effects.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Preferred unit dosage formulations are those containing a daily dose or unit, daily sub-dose, as herein discussed, or an appropriate fraction thereof, of the administered ingredient.

The dosage regimen for treating a disorder or a disease with the compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art.

The amount of compound included within therapeutically active formulations according to the present invention is an effective amount for treating the disease or condition.

In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present invention has application for both human and veterinary use.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers that can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZONE.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions that are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer that is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

The pharmaceutical compositions described herein can also be administered in further combination with another agent, for example a therapeutic agent.

The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to an angiogenesis inhibitor, such as angiostatin K1-3, DL-α-Difluoromethyl-ornithine, endostatin, fumagillin, genistein, minocycline, staurosporine, and thalidomide; a DNA intercalator/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and an unclassified antitumor agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, Vismodegib (Erivedge™), and Urinary trypsin inhibitor fragment (Bikunin). The antitumor agent may be a monoclonal antibody or antibody drug conjugate, such as rituximab (Rituxan®), alemtuzumab (Campath®), Ipilimumab (Yervoy®), Bevacizumab (Avastin®), Cetuximab (Erbitux®), panitumumab (Vectibix®), and trastuzumab (Herceptin®), Tositumomab and 131I-tositumomab (Bexxar®), ibritumomab tiuxetan (Zevalin®), brentuximab vedotin (Adcetris®), siltuximab (Sylvant™), pembrolizumab (Keytruda®), ofatumumab (Arzerra®), obinutuzumab (Gazyva™), 90Y-ibritumomab tiuxetan, 131I-tositumomab, pertuzumab (Perjeta™), ado-trastuzumab emtansine (Kadcyla™) Denosumab (Xgeva®), and Ramucirumab (Cyramza™). The antitumor agent may be a small molecule kinase inhibitor, such as Vemurafenib (Zelboraf®), imatinib mesylate (Gleevec®), erlotinib (Tarceva®), gefitinib (Iressa®), lapatinib (Tykerb®), regorafenib (Stivarga®), sunitinib (Sutent®), sorafenib (Nexavar®), pazopanib (Votrient®), axitinib (Inlyta®), dasatinib (Sprycel®), nilotinib (Tasigna®), bosutinib (Bosulif®), ibrutinib (Imbruvica™), idelalisib (Zydelig®), crizotinib (Xalkori®), afatinib dimaleate (Gilotrif®), ceritinib (LDK378/Zykadia), trametinib (Mekinist®), dabrafenib (Tafinlar®), Cabozantinib (Cometriq™), vandetanib (Caprelsa®), The antitumor agent may be a proteosome inhibitor, such as bortezomib (Velcade®) and carfilzomib (Kyprolis®). Optionally, the antitumor agent is a neoantigen. Neoantigens are tumor-associated peptides that serve as active pharmaceutical ingredients of vaccine compositions that stimulate antitumor responses and are described in U.S. Pat. No. 9,115,402, which is incorporated by reference herein in its entirety. The antitumor agent may be a cytokine such as interferons (INFs), interleukins (ILs), or hematopoietic growth factors. The antitumor agent may be INF-α, IL-2, Aldesleukin IL-2, Erythropoietin, Granulocyte-macrophage colony-stimulating factor (GM-CSF) or granulocyte colony-stimulating factor. The antitumor agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodag™) lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zolinza), Romidepsin (Istodax), or Ruxolitinib (Jakafi). For prostate cancer treatment, a preferred chemotherapeutic agent with which anti-CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone-treated murine T cell hybridomas (Nocentini et al. Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622. 2004). The observation that GITR stimulation has immunostimulatory effects in vitro and induced autoimmunity in vivo prompted the investigation of the antitumor potency of triggering this pathway. A review of Modulation Of CTLA-4 And GITR For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAG3, ICOS, PDL1 or KIR (Page et a, Annual Review of Medicine 65:27 (2014)). In further additional embodiments, the checkpoint inhibitor is targeted at a member of the TNFR superfamily such as CD40, OX40, CD137, GITR, CD27 or TIM-3. In some cases targeting a checkpoint inhibitor is accomplished with an inhibitory antibody or similar molecule. In other cases, it is accomplished with an agonist for the target; examples of this class include the stimulatory targets OX40 and GITR.

Other additional therapeutic agents include HSP inhibitors (Darby, J. and Workman, P., Chemical biology: Many faces of a cancer-supporting protein. Nature 478, 334-335 (20 Oct. 2011)). Heat shock protein 90 (HSP90) is a key component of a multichaperone complex involved in the post-translational folding of a large number of client proteins, many of which play essential roles in tumorigenesis. HSP90 has emerged in recent years as a promising new target for anticancer therapies. Tumor cells are in a stressful environment and depend on HSP90 to grow and survive (Brough P A, Aherne W, Barril X, et al. 4,5-diarylisoxazole Hsp90 chaperone inhibitors: potential therapeutic agents for the treatment of cancer. J Med Chem. 2008; 51(2):196-218.). Inhibition of heat shock proteins (HSP) intervenes in the function of a variety of oncogenic proteins important for tumor growth and survival (Brough P A, Aherne W, Barril X, et al. 4,5-diarylisoxazole Hsp90 chaperone inhibitors: potential therapeutic agents for the treatment of cancer. J Med Chem. 2008; 51(2):196-218). Such inhibitors may be NVP-AUY922, described in Breast Cancer Res. 2008; 10(2): 1996. Epub 2008 Apr. 22. The HSP inhibitor may be the oral Hsp90 inhibitor NVP-HSP990 described in Menezes et al., Mol Cancer Ther. 2012 March; 11(3):730-9. A Phase I dose-escalation, open-label study of HSP990 administered orally in adult patients with advanced solid malignancies is described in J Clin Oncol 31, 2013 (suppl; abstr 2561. The HSP inhibitor may be AUY922, a novel intravenous HSP90 inhibitor. Examples of Hsp90 inhibitors include herbimycin, geldanamycin (GA), 17-AAG e.g. Kos-953 and CNF-1010, 17-DMAG (Kos-1022), CNF-2024 (an oral purine), and IPI-504, in particular 17-AAG e.g. Kos-953 and CNF-1010, 17-DMAG (Kos-1022), CNF-2024, and IPI-504. Preferred compounds are geldanamycin analogs such as 17-AAG e.g. Kos-953 and CNF-1010, 17-DMAG (Kos-1022), and IPI-504. Other agents may be pyridine/pyrazine amide derivatives as described in international publication number WO 2013/038381.

Cell cycle progression through each phase is regulated by heterodimers formed by cyclin-dependent kinases (CDKs) and their regulatory partner proteins, the cyclins. Together they coordinate the cellular events through cell cycle. De-regulation of cell-cycle control due to aberrant CDK activity is a common feature of most cancer types. Not being bound by a theory, there may be synergy between PRMT5 inhibition and CDK inhibition. Intensive research on small molecules that target cell cycle regulatory proteins has led to the identification of many candidate inhibitors that are able to arrest proliferation and induce apoptosis in neoplastic cells as a promising strategy to treat cancer (Canavese, M., et al. (2012) Cyclin dependent kinases in cancer, Cancer Biology & Therapy, 13:7, 451-457). A number of CDK inhibitors (CDKIs) with different mechanisms of action have been evaluated. More than 50 pharmacological CDK inhibitors have been described, some of which have potent antitumor activity (Current Opinion in Pharmacology, 2003(3): 362-370). A comprehensive review about the known CDK inhibitors may be found in Angew. Chem. Int. Ed. Engl. 2003, 42(19):2122-2138. In certain preferred embodiments, PRMT5 inhibition and CDK inhibition are implemented as part of a combination therapy to arrest proliferation and induce apoptosis in neoplastic cells. CDK inhibitors for use in the present invention include, but are not limited to Palbociclib (PD-0332991) HCl, Roscovitine (Seliciclib, CYC202), UCN-01, SNS-032 (BMS-387032), Dinaciclib (SCH727965), Flavopiridol (Alvocidib), AT7519, Flavopiridol (Alvocidib) HCl, JNJ-7706621, AZD5438, MK-8776 (SCH 900776), PHA-793887, BS-181 HCl, Palbociclib (PD0332991), Isethionate A-674563, LY2835219, BMS-265246, PHA-767491, Milciclib (PHA-848125), R547, NU6027, P276-00, AT7519 HCl, Purvalanol A, Ro-3306, SU9516, XL413 (BMS-863233, LDC000067, ML167, TG003, Ribociclib (LEE011), and ZK 304709.

In an aspect, the invention provides kits containing any one or more of the elements discussed herein to allow administration of the combination therapy. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit may advantageously allow the provision of all elements of the systems of the invention. Kits can involve vector(s) and/or particle(s) and/or nanoparticle(s) containing or encoding RNA(s) to be administered to an animal, mammal, primate, rodent, etc., with such a kit including instructions for administering to such a eukaryote; and such a kit can optionally include any of the anti-cancer agents described herein.

Although the present invention and its advantages have been described in detail, it should be understood that various changes, substitutions and alterations can be made herein without departing from the spirit and scope of the invention as defined in the appended claims.

The present invention will be further illustrated in the following Examples, which are given for illustration purposes only and are not intended to limit the invention in any way.

### EXAMPLES

### Example 1

Genetic Vulnerabilities Associated with MTAP Loss

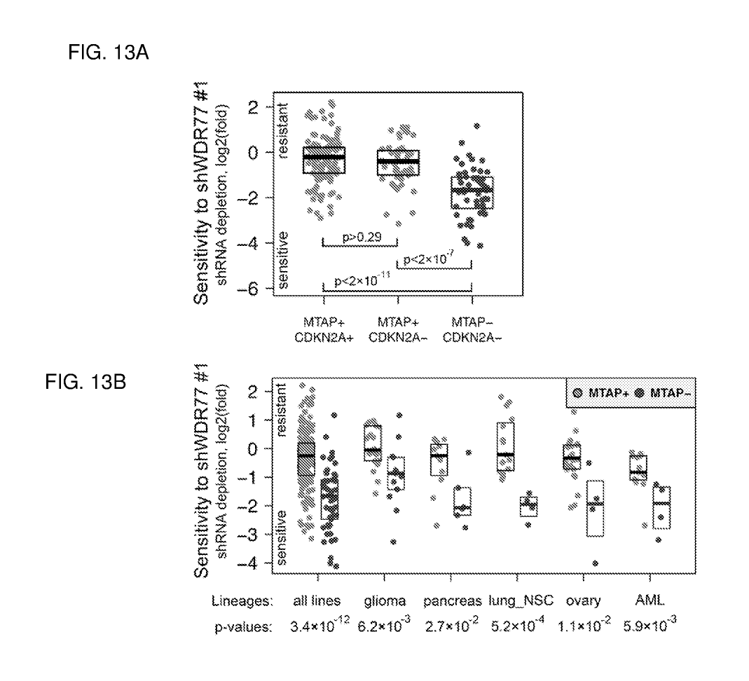

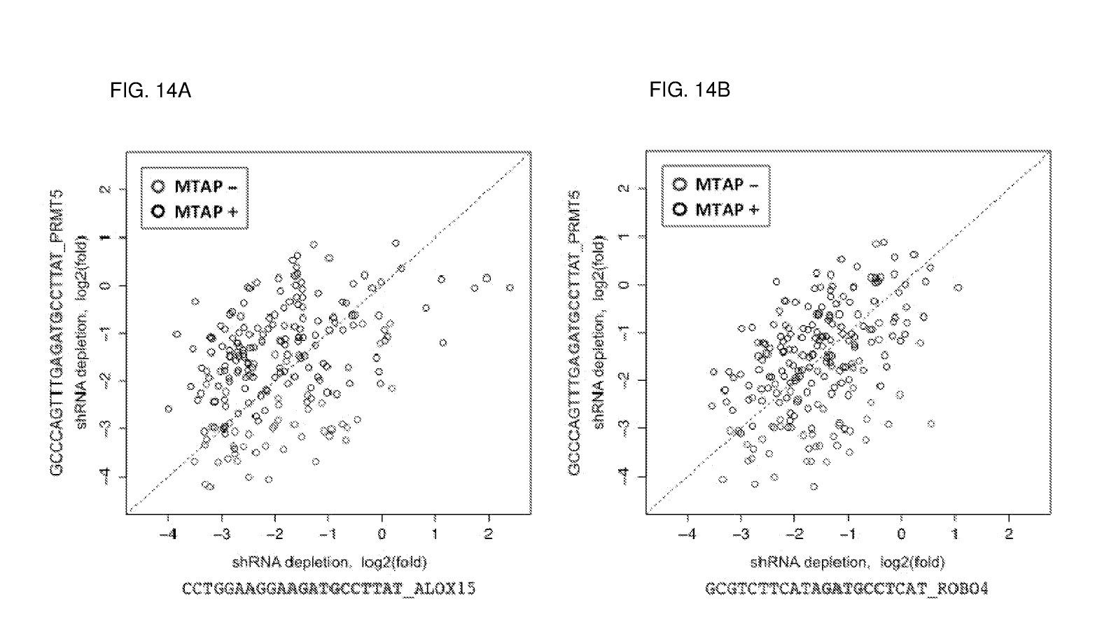

MTAP is ubiquitously expressed in normal tissues (FIG. 12) but frequently co-deleted with CDKN2A in many cancer types (FIG. 1A). Applicants searched for genetic vulnerabilities associated with MTAP loss by leveraging genome-scale pooled short hairpin RNA (shRNA) screening data for 216 cancer cell lines from Project Achilles (12, 13). MTAP deletion status for each line was determined using profiles of MTAP copy number and mRNA expression from the Cancer Cell Line Encyclopedia (CCLE) (14). Applicants correlated 50,529 shRNA sensitivity profiles with MTAP deletion status across these lines and identified two shRNAs that strongly correlated with reduced viability of MTAP-null (MTAP−) lines (n=50) but not MTAP-positive (MTAP+) lines (n=166; FIG. 1 B). One shRNA targeted PRMT5 (shPRMT5 #1; two-sided Wilcoxon p<3×10⁻¹⁵) and the other targeted WDR77 (shWDR77 #1; p<4×10⁻¹²). Applicants observed a correlation between sensitivity to these shRNAs (FIG. 1C), suggesting that MTAP− lines sensitive to suppression with either shRNA were generally also sensitive to suppression with the other shRNA. Cell lines with loss of CDKN2A but not MTAP were generally less sensitive to PRMT5 or WDR77 depletion than were lines with co-deletion of CDKN2A and MTAP, suggesting a correlation with MTAP (but not CDKN2A) loss (FIG. 1D; FIG. 13). To provide further support for a possible dependency on PRMT5 or WDR77 in the setting of MTAP loss, we examined additional shRNAs against PRMT5 and WDR77 from the screening dataset. We identified a second shRNA against PRMT5 (shPRMT5 #2) and WDR77 (shWDR77 #2) that also demonstrated a strong correlation between impaired cell viability and MTAP loss (FIG. 1 E).

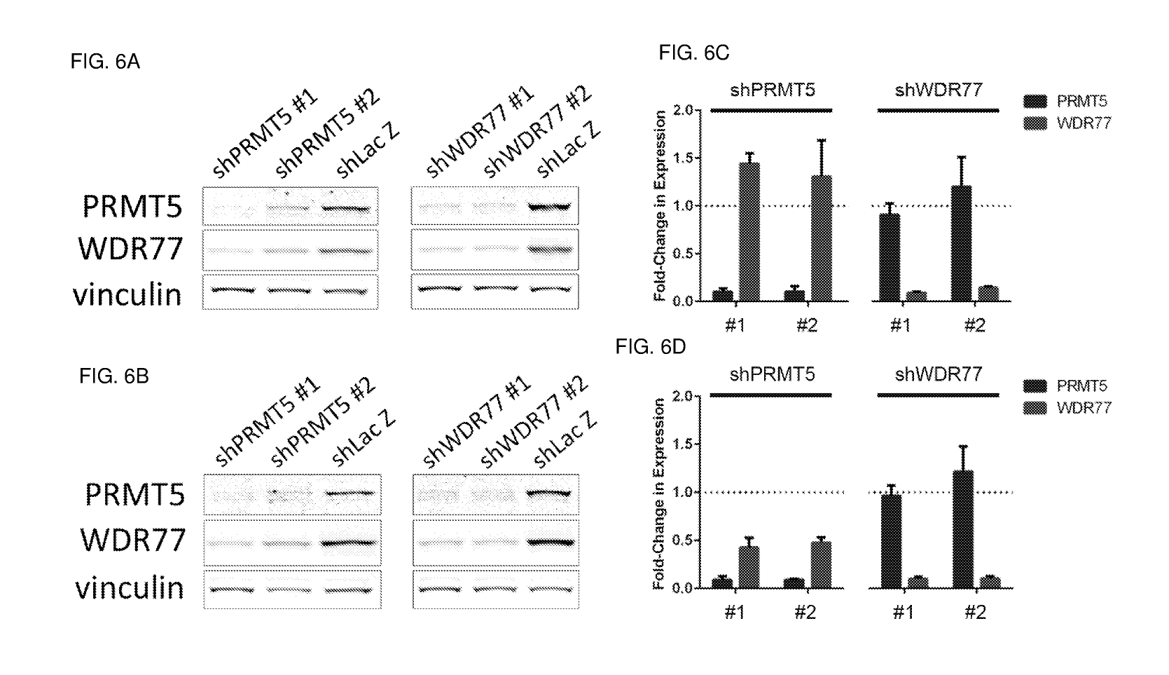

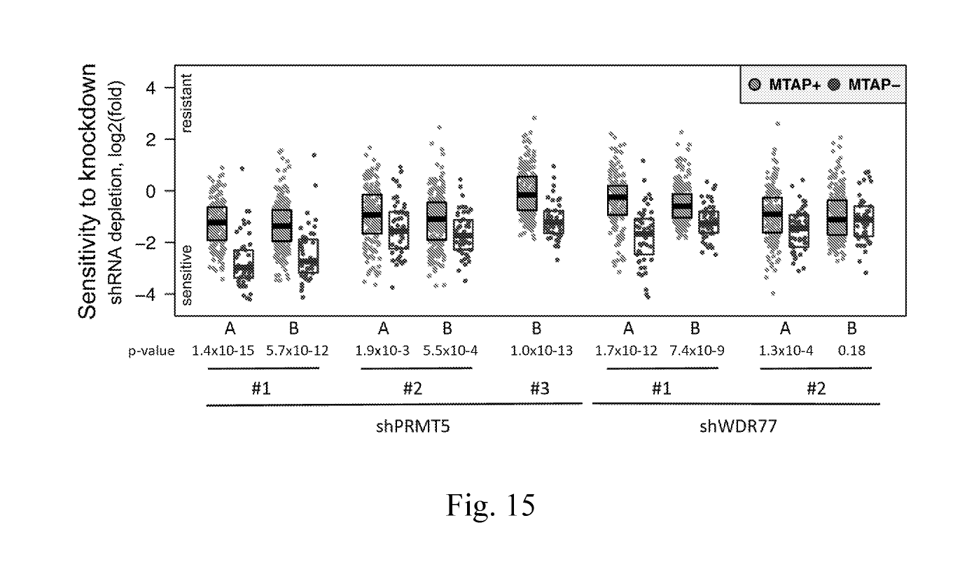

False positive findings can occur from genome-scale shRNA analyses because of “off-target” microRNA-like effects attributable to partial sequence complementarity with the 5′ end of the shRNA (known as the “seed” region)(15, 16). To investigate this possibility, Applicants identified shRNAs from the screening dataset that shared sequence identity in the seed region with each of the four shRNAs against PRMT5 or WDR77. None of the shRNAs with shared seed sequence identity demonstrated a correlation between cell viability and MTAP status comparable to that observed for the shRNAs against PRMT5 or WDR77, arguing that the differential viability was not caused by a seed effect (FIGS. 1b, 15). Applicants also confirmed on-target activity of all four shRNAs against PMRT5 or WDR77 by immunoblotting of lysates from shRNA-expressing cells (FIG. 6).

PRMT5 and WDR77 encode critical components of the methylosome. PRMT5 forms a complex with WDR77 and catalyzes the transfer of methyl groups to arginine side-chains of target proteins including histones (involved in chromatin remodeling and gene expression) and Sm proteins (RNA-binding proteins involved in mRNA processing) (17-19). Genetic depletion of PRMT5 has previously been reported to impair cancer cell viability by promoting G1 cell cycle arrest and apoptosis (20-22). Interestingly, shRNAs targeting either PRMT5 or WDR77 reduced levels of both proteins (while demonstrating specific suppression of the target transcript), consistent with depletion of the methylosome complex using either shRNA (FIG. 6). MTAP− cells were also sensitive to shRNA-mediated depletion of CLNS1A and RIOK1, which encode two additional components of the methylosome (FIG. 1E) (23). Finally, the correlation between MTAP loss and sensitivity to PRMT5 or WDR77 suppression was not confounded by cell lineage. Within individual lineages (including glioma, pancreatic adenocarcinoma, and NSCLC), MTAP− cell lines were generally (but not universally) more sensitive to depletion of PRMT5 and WDR77 than were MTAP+ lines (FIG. 1F; FIG. 13).

Based on these observations, Applicants determined that MTAP loss may confer enhanced sensitivity to genetic suppression of PRMT5 and WDR77. To validate this hypothesis, Applicants examined effects of shRNAs targeting PRMT5 and WDR77 on cell viability in 275 additional cancer cell lines profiled through Project Achilles. This profiling data was generated using an expanded shRNA library with additional shRNAs not included in the initial study. Similar to findings from the initial screening dataset, Applicants observed that MTAP− lines (n=3447) were generally more sensitive to PRMT5 or WDR77 suppression than MTAP+ lines (n=228; FIG. 1 G). Three of the four shRNAs used to establish our initial finding from the screening dataset again demonstrated a strong correlation between loss of cell viability and MTAP status, as did an additional shRNA targeting PRMT5 not included in the screening dataset (shPRMT5 #3). In total, the overall increased sensitivity of MTAP− cells to PRMT5 or WDR77 depletion was demonstrated with five shRNAs (three targeting PRMT5 and two targeting WDR77) from two independent functional datasets comprising 491 cancer cell lines (FIG. 15).

### Example 2

Cell Viability Associated with MTAP Loss

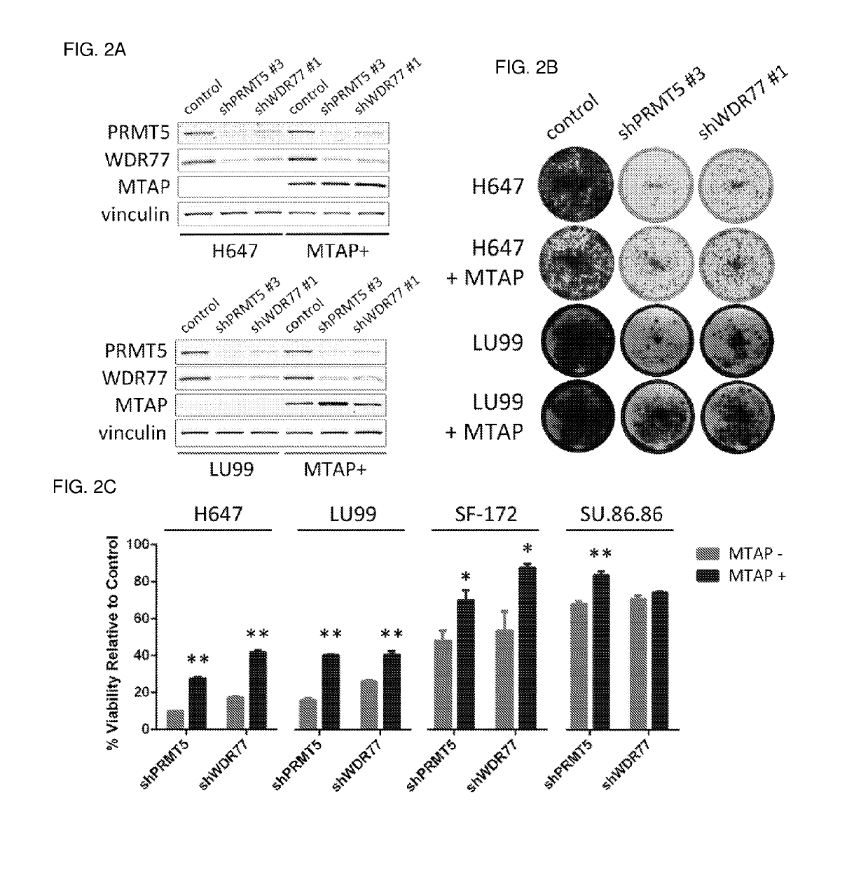

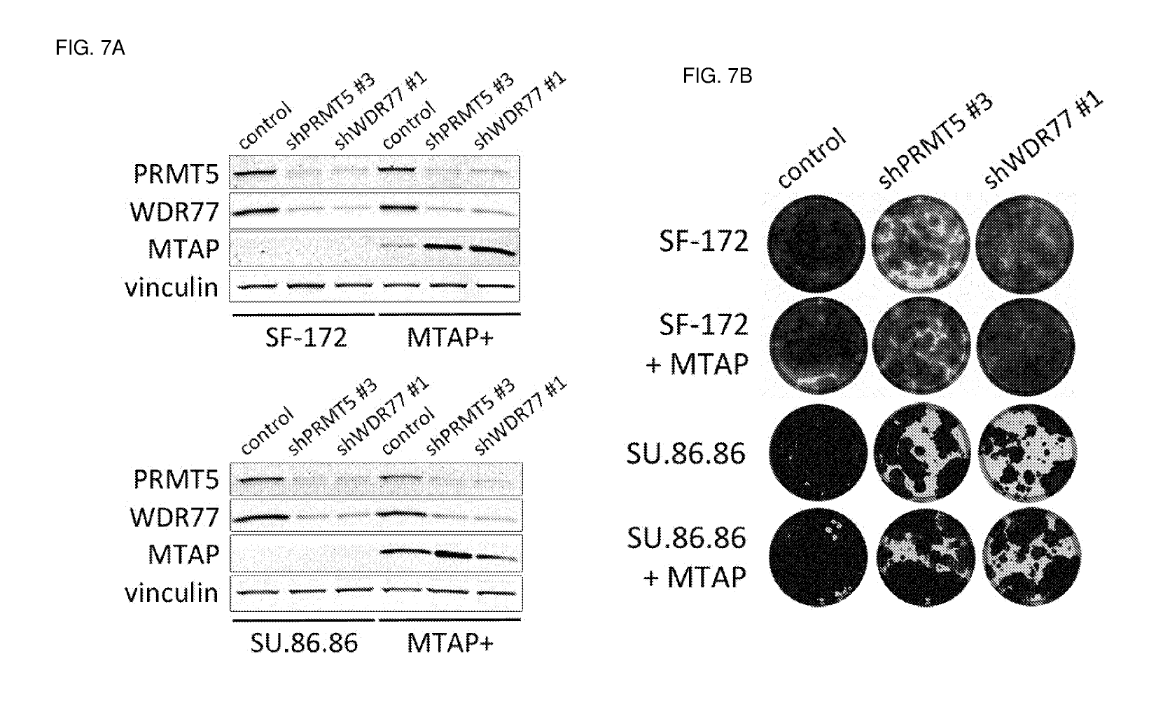

To determine whether the effects of PRMT5 or WDR77 suppression on cell viability are affected by MTAP, Applicants first introduced MTAP into four MTAP− cell lines [LU99 and H647 (NSCLC), SF-172 (glioma), and SU.86.86 (pancreatic ductal carcinoma)]. This resulted in robust MTAP protein expression in MTAP-reconstituted lines, whereas MTAP was absent from parental lines (FIG. 2A, FIG. 7). Applicants then performed colony formation assays to assess differences in cell viability following depletion of PRMT5 or WDR77 in the presence or absence of MTAP. Applicants observed a reduction in cell viability for each MTAP− cell line with PRMT5 or WDR77 suppression, consistent with our screening and validation results (FIG. 2B, C; FIG. 7). Overall, MTAP-reconstituted derivative lines demonstrated reduced sensitivity to PRMT5 or WDR77 suppression compared to their isogenic MTAP− counterparts, suggesting a functional link between MTAP loss and PRMT5 orWDR77 dependency (FIG. 2B, C; FIG. 7).

### Example 3

### PRMT Proteins are Inhibited by MTA and MTAP-Null Cancer Cells Contain Elevated MTA Levels

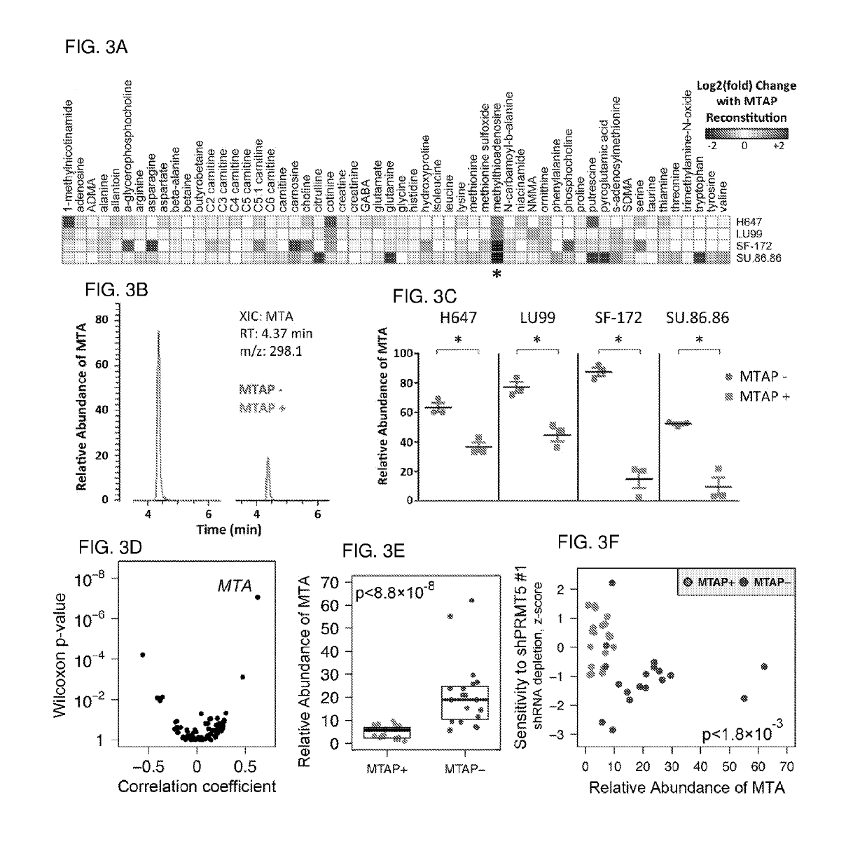

Prior studies suggest that the activity of PRMT proteins may be inhibited by MTA (the substrate of MTAP) (24, 25). MTA is an analog of S-adenosyl methionine (SAM; the donor substrate for PRMT-mediated methylation) (26) Applicants determined that somatic MTAP loss may lead to increased intracellular MTA concentrations, which in turn confers a partial inhibition of PRMT5 activity. Together, these effects may heighten cell sensitivity to further reductions in PRMT5 activity (e.g., through genetic suppression). To test this hypothesis, Applicants first determined whether MTAP−-cells contain elevated MTA levels. Applicants used liquid chromatography tandem mass spectrometry (LC-MS) to quantify levels of 56 metabolites (including MTA) from LU99, H647, SF-172, and SU.86.86 cells and their isogenic MTAP-reconstituted counterpart lines The abundance of most measured metabolites was not significantly altered by ectopic MTAP reconstitution (FIG. 3A). However, intracellular MTA abundance was reduced by 1.5 to 6-fold with MTAP reconstitution in each isogenic cell line pair, consistent with increased intracellular MTA in the absence of MTAP (FIG. 3A-C).

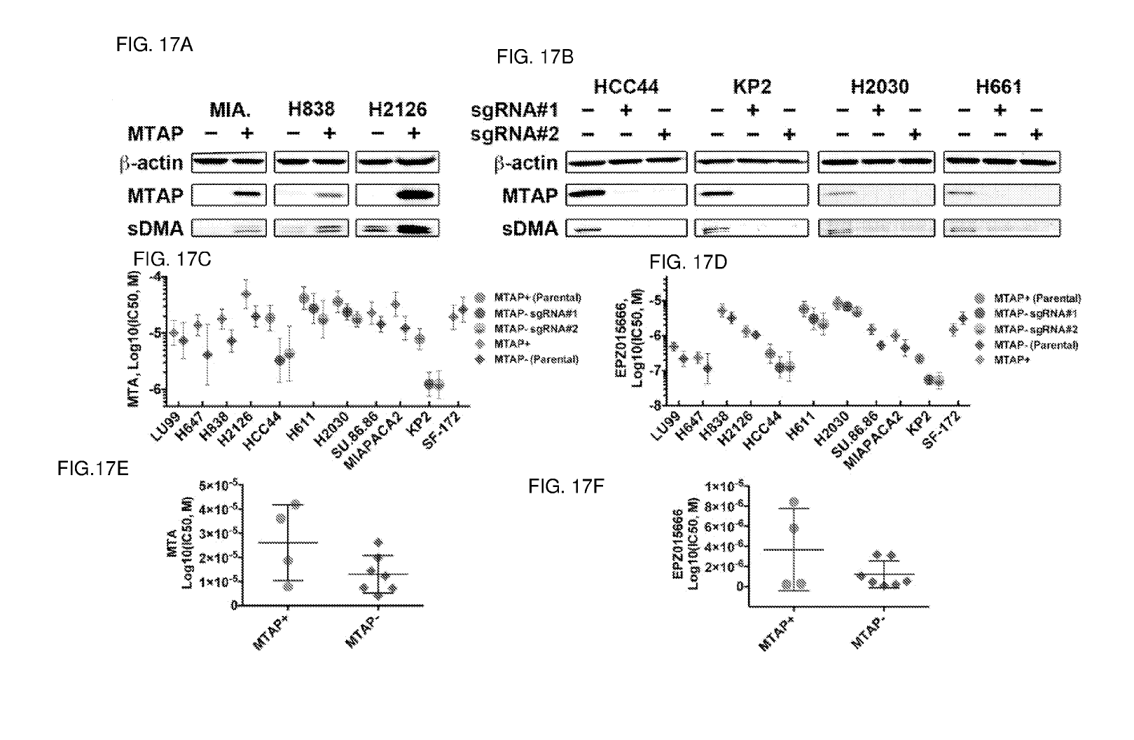

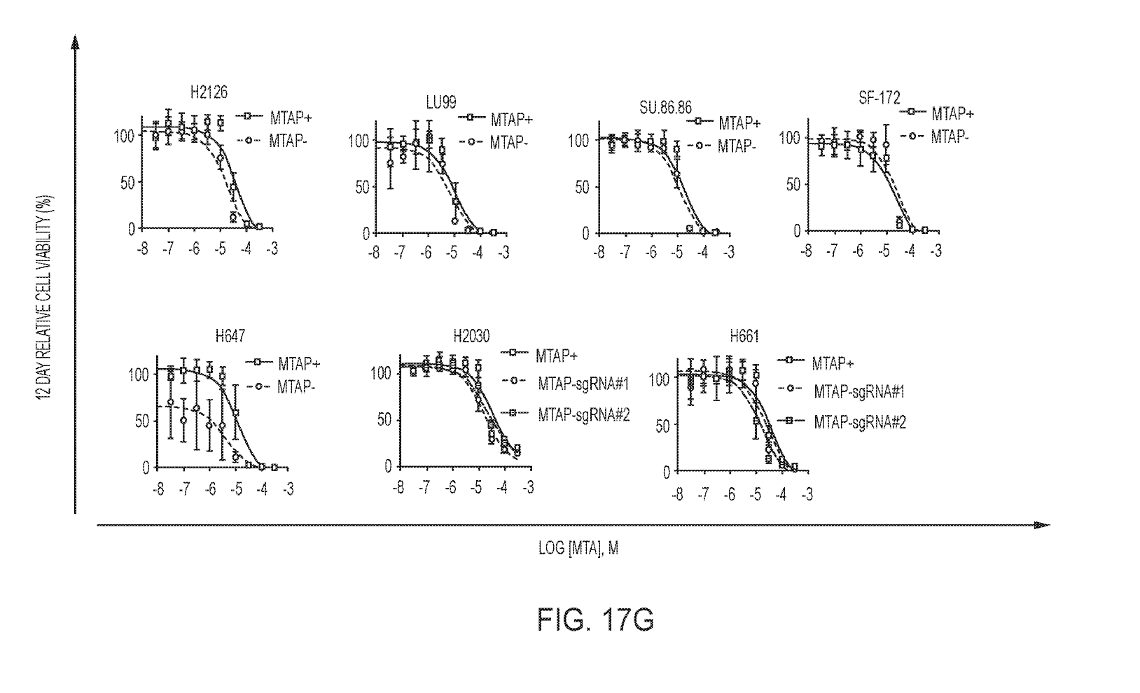

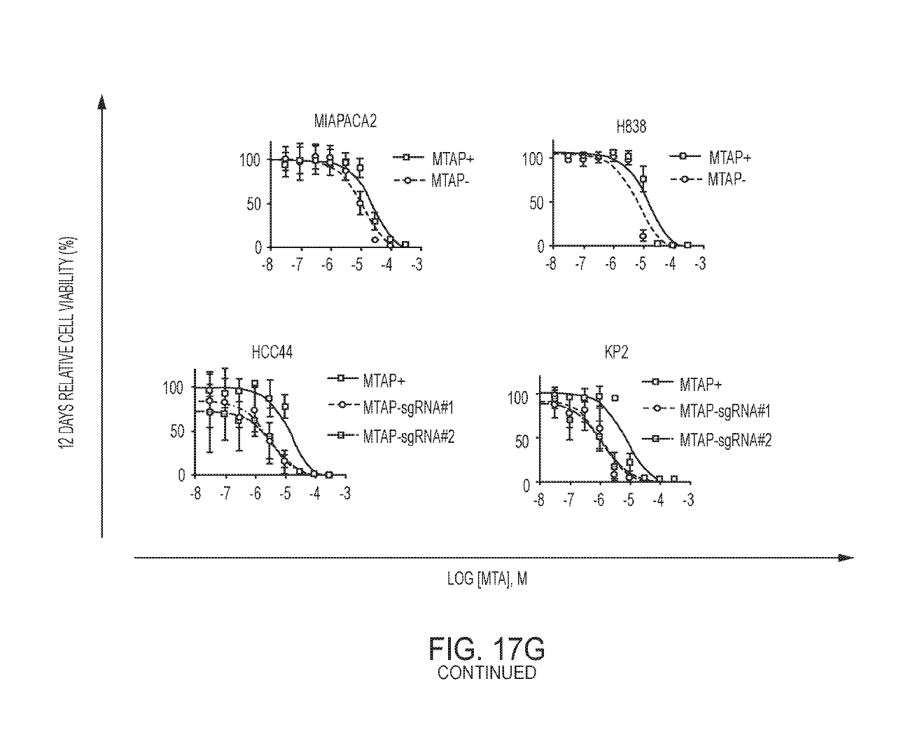

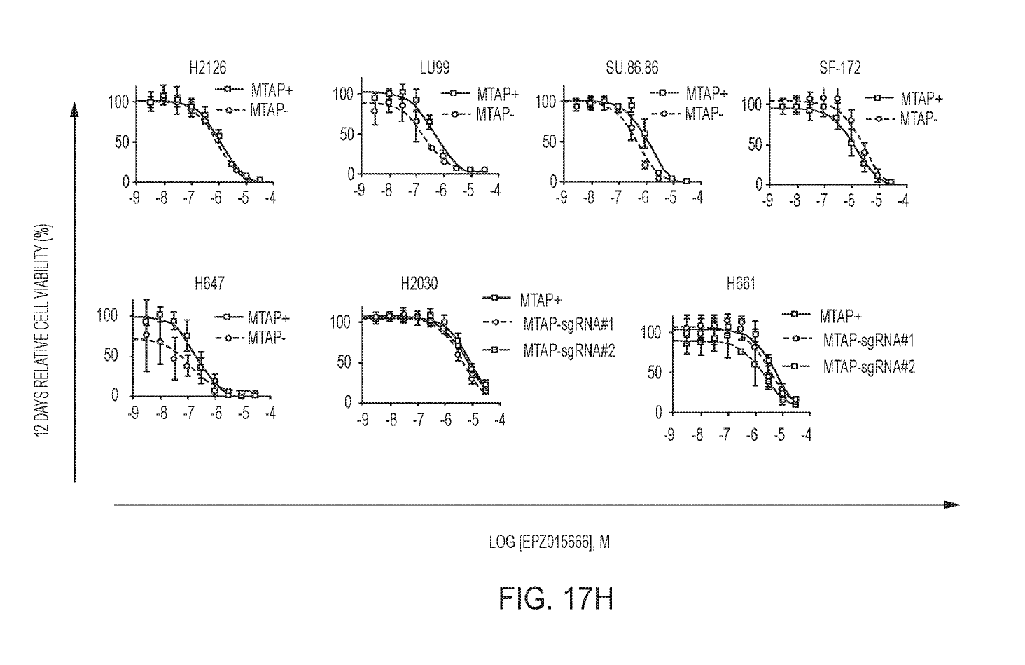

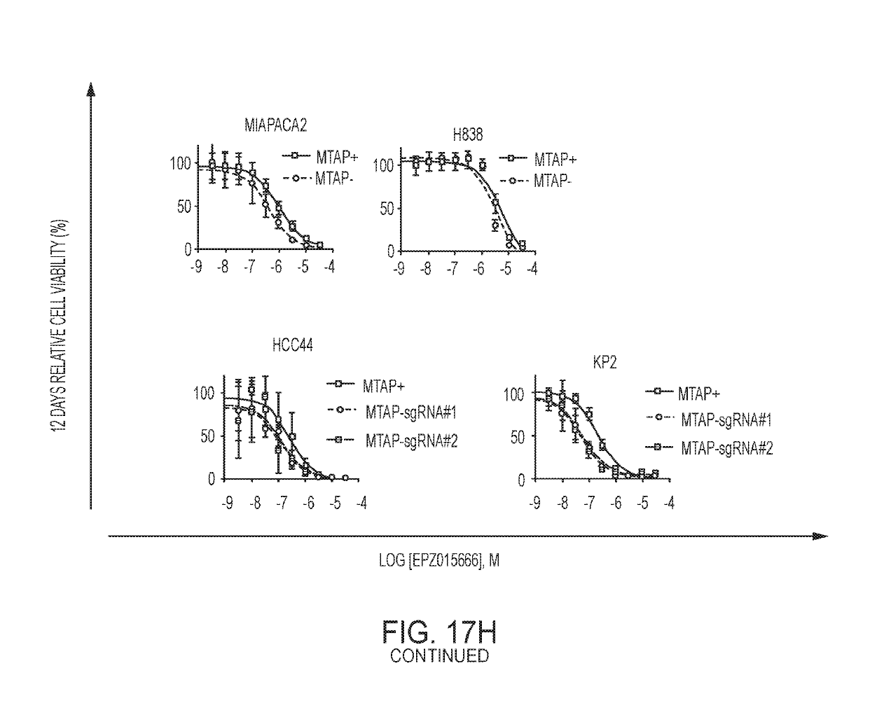

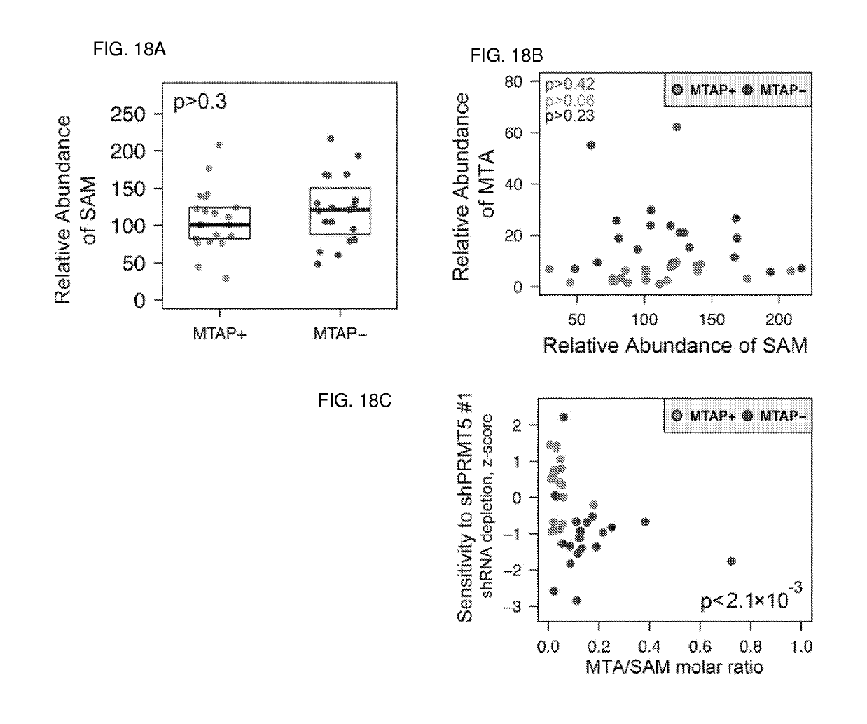

To determine whether MTA levels are generally higher in MTAP− cell lines compared to MTAP+ lines, Applicants quantified intracellular levels of 73 metabolites from MTAP− (n=19) and MTAP+(n=21) cancer cell lines from various lineages including NSCLC, melanoma, and breast. Among profiled metabolites, the abundance of MTA was most strongly correlated with MTAP loss (FIG. 3D). Applicants observed an approximately 3.3-fold increase in median MTA levels in MTAP− lines compared to MTAP+, consistent with the hypothesis that MTAP loss leads to increased intracellular MTA (FIG. 3E). In contrast, intracellular levels of the methyl donor SAM were not significantly different between MTAP− and MTAP+ lines (FIG. 18). Using shRNA sensitivity data from Project Achilles, Applicants also observed a significant correlation between MTA levels and PRMT5 dependency across profiled cell lines (FIG. 3F; FIG. 17).

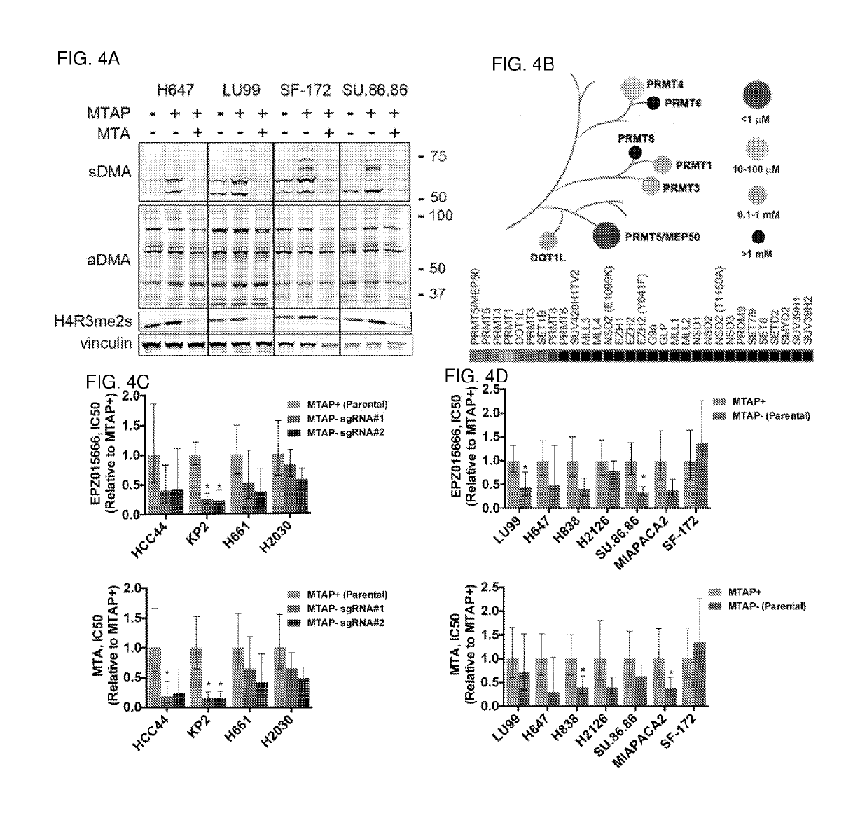

Next, Applicants assessed whether elevated MTA might inhibit PRMT5 activity. PRMT5 catalyzes the formation of symmetric dimethyl arginine (sDMA), while most other PRMTs generate asymmetric dimethyl arginine (aDMA) (17, 27, 28). Using an antibody previously shown to recognize sDMAs generated by PRMT5 (29), Applicants observed decreased sDMA levels in MTAP− cells compared to isogenic, MTAP-reconstituted lines (FIG. 4A). In addition, reduced sDMA was observed in MTAP-reconstituted cells exposed to exogenous MTA, consistent with inhibition of PRMT5 enzymatic activity (FIG. 4A). Similar findings were observed with an antibody recognizing symmetric methylation of histone H4 arginine 3 (H4R3), an established substrate of PRMT5 (FIG. 4A) (30). In contrast, Applicants observed only modest effects of MTAP status or exogenous MTA on levels of aDMA (FIG. 4A).

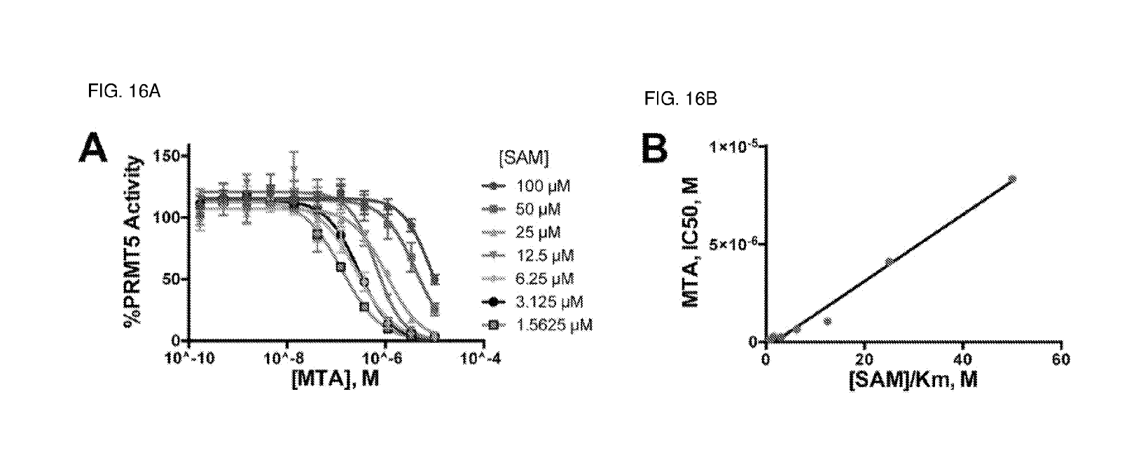

This finding raised the possibility that among PRMT family members, PRMT5 may exhibit heightened sensitivity to MTA intracellular concentrations. To test this, Applicants measured the ability of MTA to inhibit the catalytic function of 31 histone methyltransferases (including PRMT5 and the PRMT5/WDR77 complex) using a radioisotope filter binding assay(31). Applicants observed more than 100-fold selectivity for MTA against both PRMT5 and PRMT5/WDR77 activity compared to all other profiled methyltransferases, consistent with the hypothesis that PRMT5 function is selectively vulnerable to elevated MTA concentrations (FIG. 4B). Furthermore, Applicants demonstrated that MTA is a SAM-competitive inhibitor of PRMT5 (FIG. 16).

### Example 4

Pharmacologic Inhibition of PRMT5 is Selectively Lethal to Cancer Cells that Harbor MTAP Loss

Next, Applicants sought to determine whether MTAP− cell lines might exhibit increased sensitivity to pharmacologic inhibition of PRMT5 compared to MTAP+ lines. Applicants identified two inhibitors with distinct PRMT5 binding sites: the metabolite MTA itself and EPZ015666, a potent peptide-competitive and SAM-cooperative inhibitor with >10,000-fold specificity against PRMT5 relative to other methyltransferases (32). Applicants tested the ability of these inhibitors to selectively impair viability of parental MTAP− cell lines compared to isogenic lines expressing MTAP, as well as parental MTAP+ cell lines compared to isogenic CRISPR-mediated MTAP knockout lines (FIG. 17). Among the 11 isogenic cell line pairs assayed, the IC₅₀ values (concentrations of inhibitor that led to a 50% reduction in viability) for MTAP− cell lines treated with MTA or EPZ015666 were generally lower than IC₅₀ values for isogenic MTAP+ lines, consistent with our findings from genetic depletion of PRMT5 (although with a smaller effect size) (FIG. 4C, D). While the results for any given cell line pair were consistent using either PRMT5 inhibitor (FIG. 17), the differences between each isogenic cell line pair were generally modest and more pronounced for some cell line pairs than others (the differential sensitivity was absent altogether in SF-172). Furthermore, Applicants did not observe significant differences in mean IC₅₀ values between MTAP+ and MTAP− cell lines for either compound (FIG. 17)

The discrepancy in effect size that Applicants observed between genetic depletion and enzymatic inhibition of PRMT5 may be caused by several factors. For example, it is possible that the reported SAM-cooperative mechanism of action of EPZ015666 limits inhibition of PRMT5 in the setting of excess MTA and reduced SAM binding (32). Consistent with this, assays of PRMT5 activity in the setting of excess MTA are reported to increase the IC₅₀ of EPZ015666 by an order of magnitude (33). In addition, we cannot exclude the possibility that a non-catalytic PRMT5 function also contributes to the dependency. In this case, therapeutic approaches to exploit this type of vulnerability may require strategies that deplete protein levels of either PRMT5 itself or the larger methylosome complex. Further work will be necessary to explore these and other mechanistic possibilities.

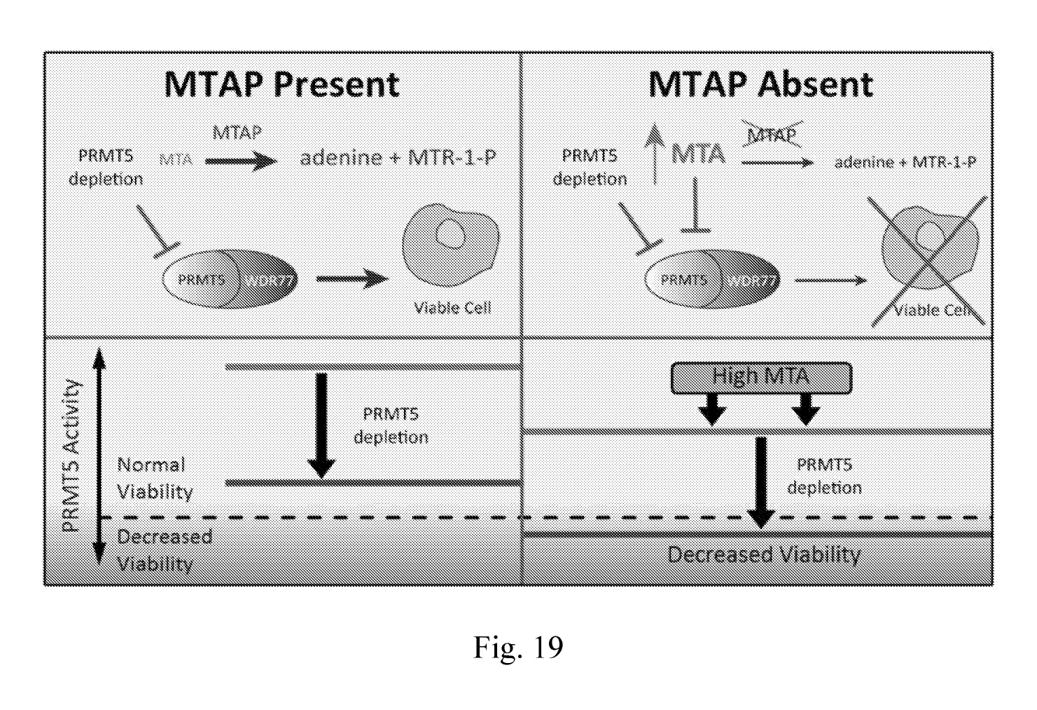

Collectively, our findings suggest that MTAP loss leads to increased intracellular MTA, which in turn inhibits PRMT5 activity and confers heightened susceptibility to further depletion of PRMT5 (FIG. 19). While PRMT5 has recently emerged as a possible therapeutic target in some cancers (26), genetic alterations correlated with sensitivity to PRMT5 inhibition have not previously been identified (26). Our data suggest that many MTAP− tumors are more sensitive to depletion of the methylosome, although there is an overlapping distribution of sensitivities to PRMT5 or WDR77 suppression between MTAP− and MTAP+ cell lines (FIG. 1D, F, G). Thus, MTAP status alone is not sufficient to distinguish cell lines that are sensitive to PRMT5 inhibition. These observations suggest the presence of other modifiers of sensitivity to methylosome depletion that function in a manner independent of MTAP status. Nevertheless, our results endorse the unexpected notion that MTAP loss confers sensitivity to PRMT5 depletion. More generally, these findings highlight the value of comprehensive functional and molecular characterization of large cancer cell line collections to promote identification of potentially targetable dependencies conferred by common genetic lesions.

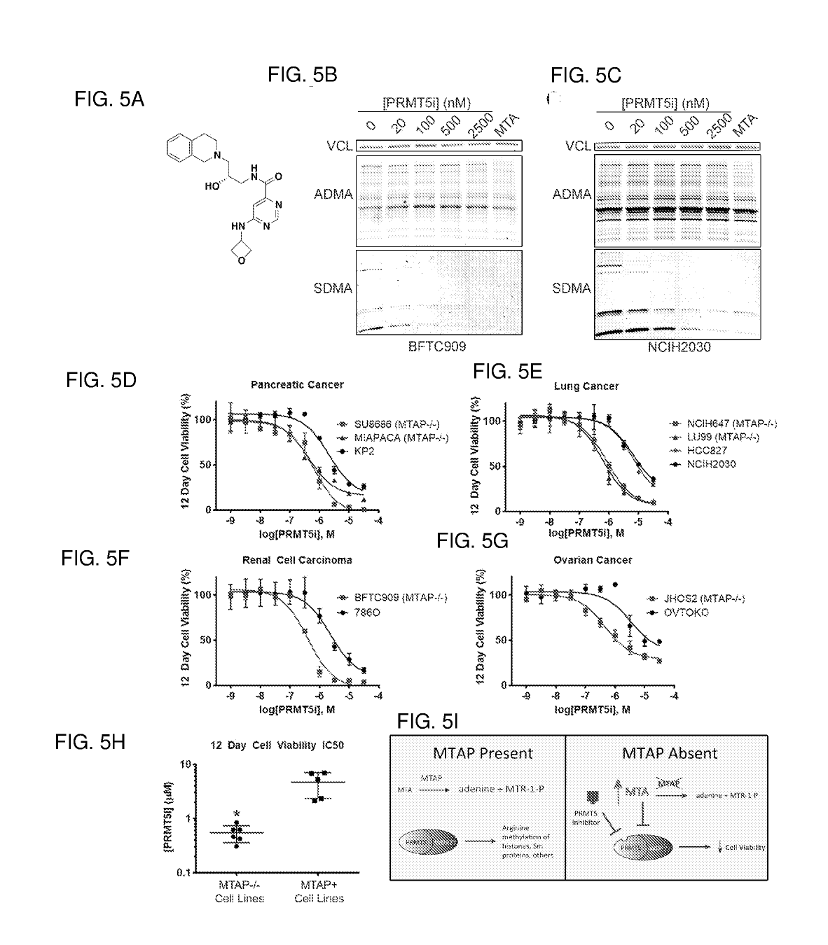

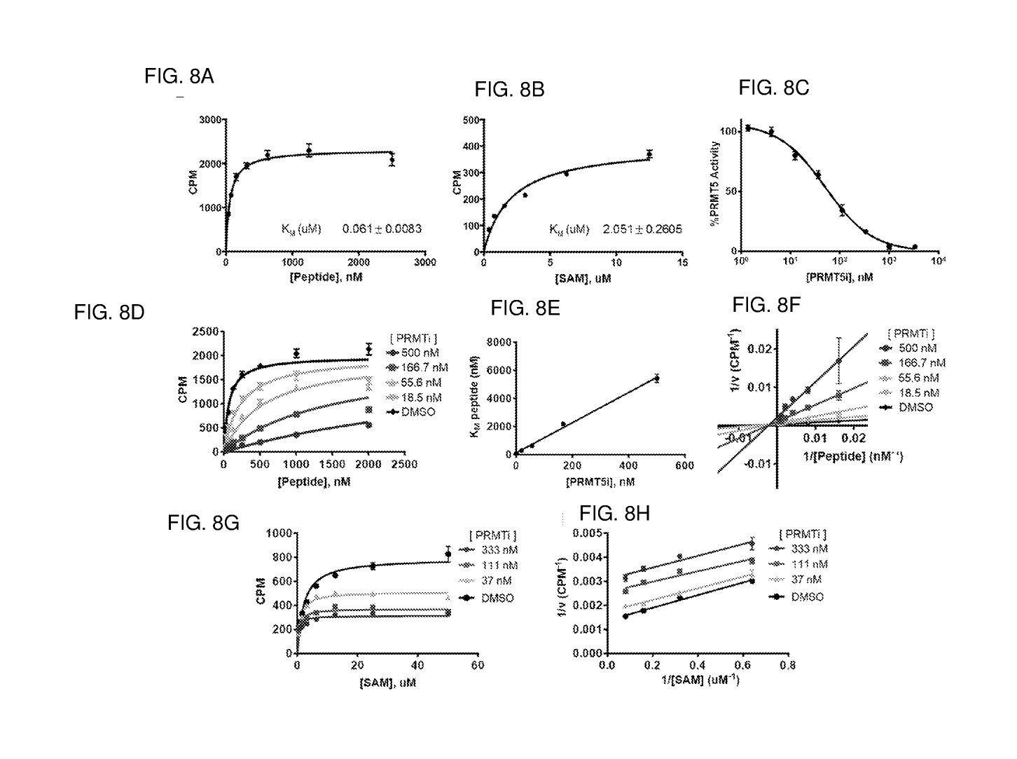

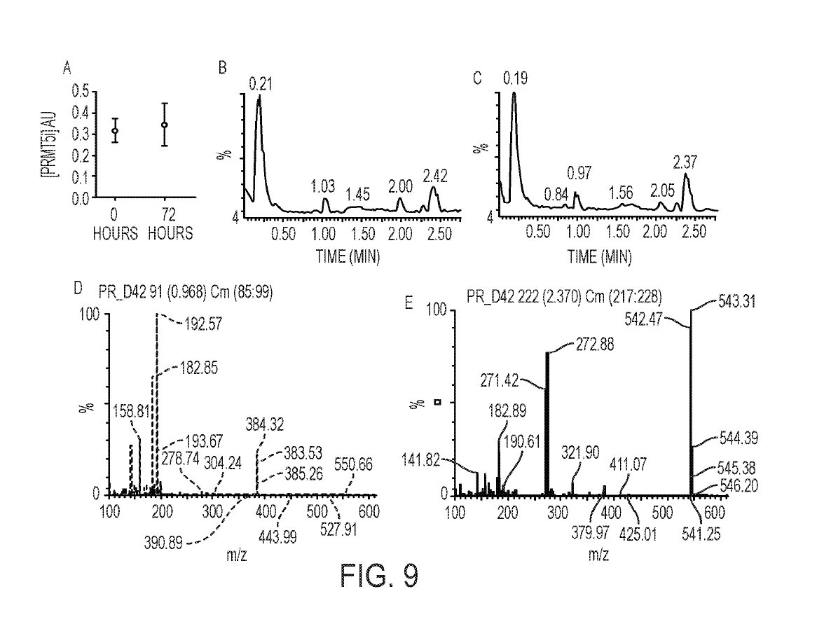

PRMT5 has previously been postulated to promote tumorigenesis in various ways; thus, efforts are underway to develop PRMT5 inhibitors. Applicants determined that selective pharmacologic inhibition of PRMT5 might prove selectively lethal to cancer cells that harbor MTAP loss. To test this hypothesis, Applicants synthesized a small molecule recently reported to inhibit PRMT5 activity in vitro (FIG. 5a). Biochemical characterization of this compound demonstrated a Ki of ˜2 nM against PRMT5 (FIG. 8), without inhibition of other histone methyltransferases at up to 50 μM (data not shown). This compound is peptide competitive and S-adenosyl methionine (SAM) uncompetitive, and thus performs optimally under the high SAM concentrations found within cells (FIG. 8e-h). The compound was stable over several days in tissue culture (FIG. 9).

To test the in vitro potency of PRMT5i against PRMT5, Applicants evaluated its effects on both sDMA and aDMA levels. PRMT5i treatment produced a marked reduction in sDMA at low nM doses, consistent with potent PRMT5 inhibition. However, the compound had no effect on aDMA production even at 2.5 μM, indicating a selective PRMT5 effect (FIG. 5a, b).

To ascertain whether MTAP− tumor cells exhibit increased sensitivity to PRMT5i compared with MTAP+ tumor cells, Applicants performed cell growth inhibition studies using a panel of MTAP− tumor cell lines (SU.86.86, MIAPACA2, NCIH647, LU99, BFTC909, JHOS2) and MTAP+ cell lines (KP2, HCC827, NCIH2030, 7860, OVTOKO) from multiple tumor types. (FIG. 5c-f). On average, MTAP− tumor cell were >8-fold more sensitive to PRMT5 inhibition than the corresponding MTAP+ lines (FIG. 5g). These data support the hypothesis that MTAP− tumor cells are sensitive to enzymatic inhibition of PRMT5 and suggest that pharmacologic PRMT5 inhibition may represent a viable approach to selectively kill MTAP− tumor cells (FIG. 5h).

The identification of genetic alterations that predict response to specific therapies can facilitate design of successful clinical trials by identifying patients likely to receive maximal benefit from a drug. MTAP deletion has long been postulated as a possible basis for “synthetic lethal” therapeutic exploitation owing to the importance of this enzyme in the adenosine salvage pathway. Conversely, PRMT5 has recently emerged as a possible target in some cancers; however, genetic alterations that predict sensitivity to PRMT5 inhibition were not previously identified. Taken together, our results endorse an unexpected but therapeutically tractable premise that homozygous MTAP loss predicts sensitivity to PRMT5 inhibition. As MTAP is ubiquitously expressed in healthy normal tissues and MTAP loss predicts sensitivity to PRMT5 inhibition across multiple cancer lineages, our results show that PRMT5 inhibition in vivo may preferentially ablate MTAP− tumor cells while sparing MTAP-expressing cells in normal tissues. More generally, these findings highlight a role for genomic perturbation screens to identify novel genetic dependencies that can serve to guide development of preclinical therapeutics.

### Example 5

### Crystal Structure of PRMT5:MEP50 Complex

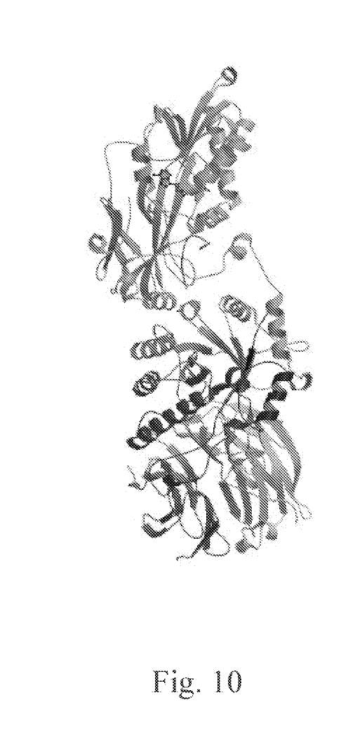

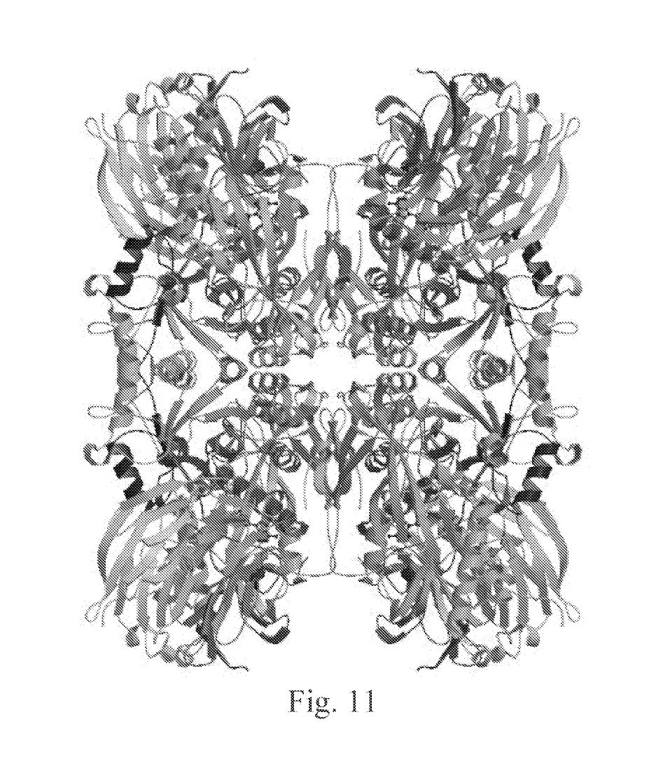

The crystal structure of the PRMT5:MEP50 complex shows regions where an inhibitor of PRMT5 activity binds. MTA binds to a different region than that of PRMT5i (FIG. 10, 11.)(www.rcsb.org/pdb/explore/explore.do?structureId=4GQB loss)

### Materials and Methods

Bioinformatic Analysis.

shRNA screening data (version 2.4.3) was downloaded from the Project Achilles portal (www.broadinstitute.org/achilles). This dataset includes 216 cancer cell lines screened with a library of 54,000 shRNAs. shRNAs profiled in less than 100 cell lines were excluded from our analysis, leaving a total of 50,529 shRNAs evaluated.

Cancer cell line genomic characterization was performed using copy number and gene expression data derived from the Cancer Cell Line Encyclopedia (CCLE). Copy number genomic alterations and Affymetrix array-based expression data are available from the CCLE portal (www.broadinsiitute.org/ccle). RNAseq BAM files and Methods describing generation of RNAseq data are available at the Cancer Genomics Hub (CGHub) CCLE portal (cghub.ucsc.edu/datasets/ccle.html). For each cell line with RNA-seq data available, genes with RPKM (‘reads per kilobase per million base pairs’) values greater than 0.5 for all constitutive exons were classified as being expressed in that line. For cell lines with only Affymetrix array-based expression data, genes with Robust Multi-array Average (RMA) expression values above 6.5 were classified as being expressed while values below 4.5 were classified as not expressed. For cell lines with only copy number data, genes with log 2 normalized values below −2 were classified as genes with homozygous deletion.

Cell Lines and Reagents.

All cell lines were obtained from the Cancer Cell Line Encyclopedia. Cell lines were cultured in base media supplemented with 10% fetal bovine serum (Gemini Bioproducts) and penicillin (100 units/mL)/streptomycin (100 μg/mL; Cellgro). For base media, H2126 was cultured in DMEM:F12 (Gibco); MIAPACA2 and SF-172 were cultured in DMEM (Cellgro); SU.86.86, H647, LU99, KP2, NCIH2030, HCC44, H661, and H838 were cultured in RPMI-1640 (Cellgro). EPZ015666 was synthesized in house based on its published structure. MTA was purchased from Sigma. All compounds were dissolved in DMSO.

Generation of MTAP-Reconstituted Cell Lines.

Clone ccsbBroad304_06601 (which contains the MTAP cDNA in a lentiviral expression vector) was obtained from the RNAi Consortium (http://www.broadinstitute.org/rnai/public). The clone expresses MTAP from the lentiviral expression vector pLX304 (http://www.addgene.org/25890) with a cytomegalovirus (CMV) promoter. pLX304 contains a blasticidin selectable marker and encodes a C-terminal V5 epitope tag. Sanger sequencing of the clone was performed to confirm sequence identity. Lentivirus was produced using HEK293T cells as described on the RNAi Consortium Portal (http://www.broadinstitute.org/rnai/public/resources/protocols).

LU99, H647, SF-172, SU.86.86, H838, MIAPACA2, and H2126 cell lines (which lack endogenous MTAP expression) were seeded in 6-well plates. The following day, cells were spin-infected with lentivirus harboring the MTAP cDNA at 2250 rpm for 30 minutes. 24 hours after infection, virus was removed and replaced with standard growth medium containing blasticidin for selection of cells expressing MTAP. The concentration of blasticidin used for selection was determined empirically for each cell line. Cells were selected for 5 days and were then cultured in the absence of blasticidin. Ectopic MTAP expression was confirmed by immunoblotting.

Generation of MTAP-Knockout Cell Lines.

Plasmid lentiCRISPR v2 (https://www.addgene.org/52961/), which contains the S. pyogenes CRISPR-Cas9 gene, a guide RNA against a target gene, and a puromycin selectable marker, was obtained from the Genomic Perturbation Platform at the Broad Institute. Candidate single guide RNAs (sgRNAs) were designed and tested for the ability to knock out MTAP in puromycin-selected cells. Guide sequences with high on-target scores were identified using the Broad Institute sgRNA designer (www.broadinstitute.org/rnai/public/analysis-tools/sgrna-design). Lentivirus was produced as above.

MTAP-expressing KP2, H2030, H661, and HCC44 cell lines were stably transduced with lentivirus containing lentiCRISPR v2 with each candidate sgRNA, followed by selection with puromycin, in order to create MTAP-knockout cell lines. Analysis of MTAP knockout with immunoblotting revealed that guide sequences TCTGCCCGGGAGCTAAAACG (SEQ ID NO: 28) and GCAGTCATAATCTGTCGCCA (SEQ ID NO: 29) provided the strongest consistent decrease in MTAP levels among assayed sgRNAs. These guides were designated sgRNA #1 and sgRNA #2, respectively.

shRNAs. Lentiviral shRNA reagents used for validation studies were obtained from the RNAi Consortium shRNA collection (www.broadinstitute.org/rnai/public). shRNAs targeting PRMT5 included shPRMT5 #1 (target sequence GCCCAGTTTGAGATGCCTTAT (SEQ ID NO: 1)), shPRMT5 #2 (CCTCAAGAACTCCCTGGAATA (SEQ ID NO: 30)), and shPRMT5 #3 (GCGTTTCAAGAGGGAGTTCAT (SEQ ID NO: 31)). shRNAs targeting WDR77 included shWDR77 #1 (GCAAAGTGAAGTCTTTGTCTT (SEQ ID NO: 2)) and shWDR77 #2 (CAAGCCTTTCTGAGTTGTTTA (SEQ ID NO: 32)). An shRNA recognizing Lac Z (shLac Z; target TCGTATTACAACGTCGTGACT (SEQ ID NO: 33)) was used as a control in FIG. 6. pLKO vector expressing a small RNA (target ACAGTTAACCACTTTTTGAAT (SEQ ID NO: 34)) was used as a control in FIG. 2. Identity of shRNAs was verified by Sanger sequencing.

Antibodies and Immunoblotting.

Cells were lysed in 50 mM HEPES (pH 7.5), 1% Triton X-100, 10% glycerol, 150 mM NaCl, 2 mM EDTA, and 10 nM NaF with protease inhibitors (Roche) and phosphatase inhibitors (Calbiochem). Lysates were fractionated by SDS-polyacrylamide gel electrophoresis and transferred to nitrocellulose using the iBlot 2 system (Life Technologies). Two-color immunoblotting was performed using LI-COR reagents (Odyssey Blocking Buffer and IRDye 800CW and IRDye 680RD secondary antibodies) according to the manufacturer's instructions (LI-COR Biosciences). Fluorescence detection was performed using an Odyssey CLx Infrared Imaging System. Antibodies against PRMT5 (#2252), MEP50 (also known as WDR77; #2018), and MTAP (#4158) were obtained from Cell Signaling. Symmetric di-methyl arginine motif (#13222) and asymmetric di-methyl arginine motif (#13522) antibodies were also purchased from Cell Signaling. Anti-histone H4 (symmetric di-methyl R3) antibody (#ab5823) was obtained from Abcam. Anti-vinculin antibody (#V9131) was obtained from Sigma.

RNA Isolation and Real-Time PCR.

SF-172 or SU.86.86 cells were seeded in 6-well plates (50,000 cells per well for SF-172 or 150,000 cells per well for SU.86.86). The following day, cells were spin-infected with lentivirus harboring shRNAs targeting PRMT5, WDR77, or Lac Z (control) in the presence of 4 μg/mL polybrene. Virus was removed after 5 hours and replaced with standard growth media. The next day, selection for transduced cells was performed with 2 μg/mL puromycin. After 48 hours, media containing puromycin was replaced with standard growth media. Five days after infection, RNA was isolated using the RNeasy Mini kit (Qiagen). cDNA preparation was performed using the Superscript VILO cDNA synthesis kit (Life Technologies). Real-time quantitative PCR was performed using a QuantStudio 6 Flex Real-Time PCR System (Applied Biosystems) according to the manufacturer's instructions. PCR reactions were performed in triplicate using Taqman Gene Expression Assays to amplify PRMT5 (#Hs01047356_m1; Life Technologies), WDR77 (#Hs01064618_m1; Life Technologies), and HPRT1 as a control (#Hs02800695_m1; Life Technologies) at 95° C. for 10 minutes followed by 40 cycles at 95° C. for 15 seconds and 60° C. for 60 seconds. Results were quantitated using the comparative Ct method to calculate 2^{-ΔΔCt} (normalized target gene expression level). Relative amount of mRNA for each sample was normalized to HPRT1.

Colony Formation Assays.

Cells were seeded in 12-well plates at a density of 1000 to 7500 cells per well. Optimal cell seeding density was determined empirically for each cell line. The next day, cells were spin-infected with lentivirus harboring the indicated shRNAs in the presence of 4 μg/mL polybrene. Virus was removed after 5 hours and replaced with standard growth media. Cells were cultured for 10 to 18 days following lentiviral infection with media change every 3 days. Cells were then fixed with 4% formaldehyde and stained with 0.5% crystal violet. Cells were photographed using a Leica microscope and imaging software. Quantification of crystal violet uptake for each sample was performed by de-staining cells with 10% acetic acid and measurement of absorbance at 595 nm using a SpectraMax 190 instrument.

Some experimental replicates were performed by first seeding cells in 6-well plates (at a density of 50,000 to 150,000 cells per well) followed by spin-infection with lentivirus in the presence of polybrene the following day. On the day after infection, media was replaced with standard growth media containing 2 μg/mL puromycin to select for cells successfully transduced with lentivirus. After 48 hours, cells were trypsinized, counted, and re-seeded into 12-well plates at a density of (5,000 to 10,000 cells per well). Cells were then cultured for 7 to 9 days with media change every 3 days before fixation and crystal violet staining.

Quantification of Intracellular MTA Levels by Mass Spectrometry.

Cells were seeded in 6-well plates and cultured to confluence. Cells were washed with cold PBS followed by addition of 80% methanol (LC-MS grade). Plates were then incubated at −80° C. for 15 minutes. Plates were scraped to dislodge cells, and lysates were transferred to an Eppendorf tube. Lysates were centrifuged to pellet cell debris, and supernatants were transferred to a new Eppendorf tube. Cell pellets were resuspended with 80% methanol and centrifuged again. Pooled extracts were stored at −80° C.

Metabolite levels were measured using a liquid chromatography tandem mass spectrometry (LC-MS) method operated on a Nexera X2 U-HPLC (Shimadzu) coupled to a Q Exactive Plus mass spectrometer (Thermo Fisher Scientific). Mass and retention time of MTA was confirmed against an authentic reference standard. Cell extracts (10 μL) were diluted using of nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). The samples were centrifuged (10 min, 9,000×g, 4° C.) and the supernatants (10 μl) were injected onto a 150×2.1 mm Atlantis hydrophilic interaction liquid chromatography column (HILIC; Waters). The column was eluted isocratically at a flow rate of 250 μL/min with 5% mobile phase A (10 mM ammonium formate and 0.1% formic acid in water) for 0.5 minutes followed by a linear gradient to 40% mobile phase B (acetonitrile with 0.1% formic acid) over 10 minutes. Full scan, positive ion mode MS data were acquired over m/z 70-800 at 70,000 resolution and 3 Hz data acquisition rate. Additional MS settings were: ion spray voltage, 3.5 kV; capillary temperature, 350° C.; probe heater temperature, 300° C.; sheath gas, 40; auxiliary gas, 15; and S-lens RF level 40. Raw data were processed using TraceFinder 3.2 software (Thermo Fisher Scientific) and visually inspected for quality of peak integration.

Three biological replicates were assayed for each sample. For the experiment comparing metabolite levels across 40 cell lines, 73 metabolites were profiled in all lines. Applicants normalized metabolite profiling data to correct for differences in cell biomass across samples. First, Applicants divided each raw measured metabolite value by the median measured value of that metabolite across all cell lines and replicates. For each replicate of each cell line, Applicants next determined the median of these ratios for all metabolites measured from that replicate to generate a normalization factor. Applicants next divided each raw measured metabolite value by these normalization factors and determined the mean of the 3 normalized replicate values for each metabolite from each cell line. As these values represent relative metabolite abundance, for convenience all metabolite values were normalized to the lowest observed value of that metabolite across cell lines. Relative molar ratios of MTA to SAM were estimated using external reference standards.

PRMT5 Methyltransferase Assay.

PRMT5 activity was measured using a radiometric Scintillation Proximity Assay (SPA) performed in 384-well OptiPlates (Perkin Elmer). For PRMT5i IC50 determination, 30 nM PRMT5/MEP50 expressed in HEK293 (BPS Bioscience, #51045) was incubated for 2 hrs at RT with 1 uM histone H4 (1-21)-lys(biotin) (Anaspec), 1.5 uM SAM (NEB), and 500 nM 3H-SAM in 20 uL reaction buffer (20 mM sodium phosphate pH 8.5, 1 mM EDTA, 1 mM TCEP, and 0.01% Tween-20) containing compound or DMSO. Reactions were quenched with TCA and, following the addition of PVT streptavidin-coated SPA beads (Perkin Elmer; 40 uL of 140 ng diluted in PBS), incubated for 1 hr at RT. CPM values were measured using the TopCount NXT plate reader. Percent activity values were calculated by setting the average background (no enzyme wells) to 0% the average DMSO wells to 100% activity. Standard deviations were determined from four replicate measurements for compound concentration. Data were analyzed and plotted using GraphPad PRISM v6 and IC50 values were determined using the ‘log(inhibitor) vs normalized response-variable slope’ analysis module.

Mechanism of Action (MOA) Studies.

PRMT5 activity was measured using AlphaLISA performed in 384-well AlphaPlates (Perkin Elmer). For MTA IC50 determination, 30 nM PRMT5/MEP50 expressed in HEK293 (BPS Bioscience, #51045) was incubated for 2 hrs at RT with 1 μM histone H4 (1-21)-lys(biotin) (Anaspec), in varying SAM concentration in 20 μL reaction buffer (20 mM sodium phosphate pH 8.5, 1 mM EDTA, 1 mM TCEP, and 0.01% Tween-20) containing compound or DMSO. Reactions were quenched with the addition 20 μL detection solution containing Streptavidin Donor Beads and AlphaLISA® Anti-methyl-Histone H4 Arginine 3 Acceptor Beads diluted to 20 ng/μL in 1× Epigenetics Buffer (Perkin Elmer). After 1 hr incubation at RT, luminescence was measured on the Envision 2104 plate reader. Percent activity values were calculated by setting the average background (no enzyme wells) to 0% and the average DMSO wells to 100% activity. Standard deviations were determined from four replicate measurements for compound concentration. Data were analyzed and plotted using GraphPad PRISM v6 and IC50 values were determined using the ‘log(inhibitor) vs normalized response-variable slope’ analysis module.

Methyltransferase Selectivity Assays.

The inhibitory activity of MTA against the catalytic activity of 31 histone methyltransferases (including histone lysine methyltransferases and histone arginine methyltransferases) was assayed using the HotSpotSM radioisotope filter-binding platform (Reaction Biology Corp) as described in (31). Here, MTA was incubated in the presence of a histone methyltransferase, substrate, and tritium-labeled SAM, and detection of the methylated radiolabeled reaction product was performed using a filter-binding method. Briefly, an MTA stock solution was prepared in DMSO at 100 mM. MTA was tested in 10-dose titrations with 3-fold serial dilution starting at 3 mM. The methyltransferase inhibitors SAH (5-(5′-Adenosyl)-L-homocysteine) and chaetocin were used as positive controls; these were tested in 10-dose titrations with 3-fold serial dilution starting at 100 or 200 μM, respectively. All reactions were carried out with 1 μM tritium-labeled SAM and 5 μM peptide or protein substrate. Details about the identity and source of profiled methyltransferases and corresponding substrates are available in Additional Data Table S8 (G. V. Kryukov et al. 2016). Assays were performed in one of four buffers: Buffer A (50 mM Tris-HCl, pH 8.5, 5 mM MgCl2, 50 mM NaCl, 0.01% Brij35, 1 mM DTT), Buffer B (50 mM Tris-HCl, pH 8.0, 1 mM EDTA, 0.01% Brij35, 1 mM DTT), Buffer C (50 mM Bicine, pH 8.5, 5 mM MgCl2, 50 mM NaCl, 0.01% Brij35, 1 mM DTT), or Buffer D (50 mM Tris-HCl, pH 8.5, 0.01% Brij35, 1 mM DTT). Buffers used for each enzyme assay are listed in Additional Data Table S8 (G. V. Kryukov et al. 2016). Reactions were performed for 1 hr at 37° C. Curve fits and IC₅₀ determination were performed as described in (31). The dendrogram shown in FIG. 4B was generated using Reaction Biology's HMT Mapper (www.reactionbiology.com/webapps/site/HMTMapper.aspx?map=Methyltransferase).

MTA Mechanism of Action Study.

PRMT5 activity was measured using AlphaLISA performed in 384-well AlphaPlates (Perkin Elmer). For MTA IC₅₀ determination, 30 nM PRMT5/MEP50 expressed in HEK293 (BPS Bioscience, #51045) was incubated for 2 hrs at RT with 1 μM histone H4 (1-21)-lys(biotin) (Anaspec), using varying SAM concentration in 20 μL reaction buffer (20 mM sodium phosphate pH 8.5, 1 mM EDTA, 1 mM TCEP, and 0.01% Tween-20) containing compound or DMSO. Reactions were quenched with the addition 20 μL detection solution containing Streptavidin Donor Beads and AlphaLISA® Anti-methyl-Histone H4 Arginine 3 Acceptor Beads diluted to 20 ng/μL in 1× Epigenetics Buffer (Perkin Elmer). After 1 hr incubation at RT, luminescence was measured on the Envision 2104 plate reader. Percent activity values were calculated by setting the average background (no enzyme wells) to 0% and the average DMSO wells to 100% activity. Standard deviations were determined from four replicate measurements for compound concentration. Data were analyzed and plotted using GraphPad PRISM v6 and IC₅₀ values were determined using the ‘log(inhibitor) vs normalized response-variable slope’ analysis module.

Cell Viability IC₅₀ Measurement.

Cell plating densities were determined by evaluating the number of cells required to achieve 70% confluency after 12 days in culture. Cells were seeded in 100 μL of media in 96-well plates (excluding wells in the first or last row or column) using the following densities in cells per well: LU99 at 360, H647 at 100, SF-172 at 35, SU8686 at 250, MIAPACA2 at 135, H838 at 73, H2126 at 1042, HCC44 at 62, KP2 at 292, H661 at 166, and H2030 at 250. Isogenic cell line pairs were seeded at the same densities for all experiments.

MTA and EPZ015666 were dissolved in DMSO at 100 mM and 10 mM, respectively. Drug was administered using the HP D300 digital dispenser (Hewlett-Packard). DMSO concentration did not exceed 0.32%. Each drug concentration was plated in six replicates, and the MTA concentration range was titrated logarithmically over 316 μM to 31.6 nM while EPZ015666 concentration range was titrated logarithmically over 31.6 μM to 3.16 nM. Media and drug were changed every 4 days.

Consistent with previously-published inhibitor studies with EPZ015666 (32), total cell viability was assessed after 12-19 days using Cell Titer-Glo luminescent cell viability assay (Promega). Isogenic cell line pairs were plated and cultured in parallel for the same time period. Luminescence was measured according to the manufacturer's instructions using the EnVision Multilabel Reader (Perkin Elmer). Luminescence data from each well was normalized to luminescence from wells containing untreated cells on that same plate. IC₅₀ was calculated by non-linear regression using the ‘log(inhibitor) vs. response (three parameters) analysis in GraphPad Prism 6.

### REFERENCES

1. T. Nobori, K., et al., Genomic cloning of methylthioadenosine phosphorylase: a purine metabolic enzyme deficient in multiple different cancers. Proc Natl Acad Sci USA 93, 6203-6208 (1996).
2. Z. H. Chen, et al., Gene deletion chemoselectivity: codeletion of the genes for p16(INK4), methylthioadenosine phosphorylase, and the alpha- and beta-interferons in human pancreatic cell carcinoma lines and its implications for chemotherapy. Cancer Res 56, 1083-1090 (1996).
3. P. B. Illei, et al., Homozygous deletion of CDKN2A and codeletion of the methylthioadenosine phosphorylase gene in the majority of pleural mesotheliomas. Clin Cancer Res 9, 2108-2113 (2003).
4. S. R. Hustinx, et al., Homozygous deletion of the MTAP gene in invasive adenocarcinoma of the pancreas and in periampullary cancer: a potential new target for therapy. Cancer Biol Ther 4, 83-86 (2005).
5. E. L. Powell, et al., Concordant loss of MTAP and p16/CDKN2A expression in gastroesophageal carcinogenesis: evidence of homozygous deletion in esophageal noninvasive precursor lesions and therapeutic implications. Am J Surg Pathol 29, 1497-1504 (2005).
6. C. A. Karikari, et al., Homozygous deletions of methylthioadenosine phosphorylase in human biliary tract cancers. Mol Cancer Ther 4, 1860-1866 (2005).
7. S. A. Christopher, et al., Methylthioadenosine phosphorylase, a gene frequently codeleted with p16(cdkN2a/ARF), acts as a tumor suppressor in a breast cancer cell line. Cancer Res 62, 6639-6644 (2002).
8. E. Cerami, et al., The cBio cancer genomics portal: an open platform for exploring multidimensional cancer genomics data. Cancer Discov 2, 401-404 (2012).
9. J. R. Bertino, et al., Targeting tumors that lack methylthioadenosine phosphorylase (MTAP) activity: current strategies. Cancer Biol Ther 11, 627-632 (2011).
10. H. L. Kindler, et al., A phase II multicenter study of L-alanosine, a potent inhibitor of adenine biosynthesis, in patients with MTAP-deficient cancer. Invest New Drugs 27, 75-81 (2009).
11. P. N. Munshi, et al., 6-thioguanine: a drug with unrealized potential for cancer therapy. Oncologist 19, 760-765 (2014).
12. H. W. Cheung, et al., Systematic investigation of genetic vulnerabilities across cancer cell lines reveals lineage-specific dependencies in ovarian cancer. Proc Natl Acad Sci US A 108, 12372-12377 (2011).
13. G. S. Cowley, et al., Parallel genome-scale loss of function screens in 216 cancer cell lines for the identification of context-specific genetic dependencies. Scientific Data 1, (2014).
14. J. Barretina, G. et al., The Cancer Cell Line Encyclopedia enables predictive modelling of anticancer drug sensitivity. Nature 483, 603-607 (2012).
15. A. L. Jackson, et al., Widespread siRNA “off-target” transcript silencing mediated by seed region sequence complementarity. Rna 12, 1179-1187 (2006).
16. F. D. Sigoillot, et al., A bioinformatics method identifies prominent off-targeted transcripts in RNAi screens. Nat Methods 9, 363-366 (2012).
17. V. Karkhanis, et al., Versatility of PRMT5-induced methylation in growth control and development. Trends Biochem Sci 36, 633-641 (2011).
18. W. J. Friesen, et al., The methylosome, a 20S complex containing JBP1 and pICln, produces dimethylarginine-modified Sm proteins. Mol Cell Biol 21, 8289-8300 (2001).
19. W. J. Friesen, et al., A novel WD repeat protein component of the methylosome binds Sm proteins. J Biol Chem 277, 8243-8247 (2002).
20. A. Scoumanne, et al., PRMT5 is required for cell-cycle progression and p53 tumor suppressor function. Nucleic Acids Res 37, 4965-4976 (2009).
21. J. H. Lim, et al., PRMT5 is essential for the eIF4E-mediated 5′-cap dependent translation. Biochem Biophys Res Commun 452, 1016-1021 (2014).
22. C. M. Koh, et al., MYC regulates the core pre-mRNA splicing machinery as an essential step in lymphomagenesis. Nature 523, 96-100 (2015).
23. G. Guderian, et al., RioK1, a new interactor of protein arginine methyltransferase 5 (PRMT5), competes with pICln for binding and modulates PRMT5 complex composition and substrate specificity. J Biol Chem 286, 1976-1986 (2011).
24. M. T. Bedford, S. Richard, Arginine methylation an emerging regulator of protein function. Mol Cell 18, 263-272 (2005).
25. H. G. Williams-Ashman, et al., Trends in the biochemical pharmacology of 5′-deoxy-5′-methylthioadenosine. Biochem Pharmacol 31, 277-288 (1982).
26. Y. Yang, M. T. Bedford, Protein arginine methyltransferases and cancer. Nat Rev Cancer 13, 37-50 (2013).
27. T. L. Branscombe, et al., PRMT5 (Janus kinase-binding protein 1) catalyzes the formation of symmetric dimethylarginine residues in proteins. J Biol Chem 276, 32971-32976 (2001).
28. B. P. Pollack, et al., The human homologue of the yeast proteins Skb1 and Hs17p interacts with Jak kinases and contains protein methyltransferase activity. J Biol Chem 274, 31531-31542 (1999).
29. F. Liu, et al., Arginine methyltransferase PRMT5 is essential for sustaining normal adult hematopoiesis. J Clin Invest 125, 3532-3544 (2015).
30. Q. Zhao, et al., PRMT5-mediated methylation of histone H4R3 recruits DNMT3A, coupling histone and DNA methylation in gene silencing. Nat Struct Mol Biol 16, 304-311 (2009).
31. K. Y. Horiuchi, et al., Assay development for histone methyltransferases. Assay Drug Dev Technol 11, 227-236 (2013).
32. E. Chan-Penebre, et al., A selective inhibitor of PRMT5 with in vivo and in vitro potency in MCL models. Nat Chem Biol, (2015).
33. K. J. Mavrakis, et al., Disordered methionine metabolism in MTAP/CDKN2A deleted cancers leads to dependence on PRMT5. Science.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of treating a tumor in a subject, which comprises determining whether the level of methylthioadenosine phosphorylase (MTAP) activity is reduced in the tumor compared to a normal cell, and
if the level of MTAP activity is reduced, administering a protein arginine methyltransferase 5 (PRMT5) inhibitor in an amount effective to reduce inhibit proliferation of cells of the tumor.

2. The method of claim 1, wherein the PRMT5 inhibitor is administered in an amount that does not inhibit viability of a normal cell.

3. The method of claim 1, wherein the tumor comprises a mutation that prevents expression of MTAP.

4. The method of claim 1, wherein 5′-deoxy-5′-methylthioadenosine (MTA) or an MTA analog or MTAP inhibitor is administered with the PRMT5 inhibitor.

5. The method of claim 4, wherein the 5′-deoxy-5′-methylthioadenosine (MTA) or MTA analog or MTAP inhibitor is administered concurrently with the PRMT5 inhibitor.

6. The method of claim 4, wherein the MTA or MTA analog or MTAP inhibitor is administered separately from the PRMT5 inhibitor.

7. The method of claim 5, wherein the MTA or MTA analog or MTAP inhibitor is joined to the PRMT5 inhibitor by a linker.

8. The method of claim 7, wherein the linker is cleavable.

9. A method for identifying a tumor with increased sensitivity to an inhibitor of PRMT5, which comprises determining the level of MTAP function in the tumor.

10. The method of claim 9, which comprises determining whether the MTAP gene is deleted in tumor.

11. The method of claim 9, which comprises determining whether both copies of the MTAP gene are deleted.

12. A method of inhibiting PRMT5 in a cell comprising contacting the cell with an effective amount of an MTAP inhibitor.

13. The method of claim 12, further comprising administering an effective amount of inhibitor of PRMT5.

14. The method of claim 13, wherein the PRMT5 inhibitor is PRMT5i.

15. A method of inhibiting PRMT5 in a cell comprising contacting the cell with an effective amount of MTA or an MTA analog.

16. The method of claim 15, further comprising contacting the cell with an effective amount of an inhibitor of PRMT5.

17. The method of claim 16, wherein the PRMT5 inhibitor is PRMT5i.

18. A method of treating or preventing a PRMT5-mediated disorder in a subject, which comprises administering to subject and effective amount of 5′-deoxy-5′-methylthioadenosine (MTA) or MTA analog or an MTAP inhibitor.

19. The method of claim 18, further comprising administering an effective amount of an inhibitor of PRMT5.

20. The method of claim 19, wherein the PRMT5 inhibitor is PRMT5i.

21. The method of claim 18, wherein the disorder is a proliferative disorder, a metabolic disorder, or a blood disorder.

22. The method of claim 21, wherein the proliferative disorder is cancer.

23. The method of claim 22, wherein the cancer is hematopoietic cancer, lung cancer, prostate cancer, melanoma, or pancreatic cancer.

24. The method of claim 21, wherein the metabolic disorder is diabetes or obesity.

25. The method of claim 21, wherein the blood disorder is a hemoglobinopathy.

26. The method of claim 21, wherein the blood disorder is sickle cell anemia or β-thalassemia.

27. The method of claim 1, wherein the MTA or MTA analog bind to a site on PRMT5 that is separate from the PRMT5 inhibitor binding site.

28. The method of claim 1, wherein the MTA or MTA analog bind to a site on PRMT5 that coincides with the PRMT5 inhibitor binding site.

29. A method of identifying a suitable therapy for treatment of a tumor in a subject, which comprises measuring the level of methylthioadenosine phosphorylase (MTAP) activity in the tumor, and if the level of MTAP activity is reduced compared to normal cells, administering a protein arginine methyltransferase 5 (PRMT5) inhibitor and/or MTA to the subject, wherein the PRMT5 inhibitor and MTA inhibitor are in amounts effective to inhibit proliferation of cells of the tumor.

30. A method of identifying a suitable therapy for treatment of a tumor in a subject, which comprises measuring the level of MTA in the tumor, and if the level of MTA is increased compared to normal cells, administering a protein arginine methyltransferase 5 (PRMT5) inhibitor to the subject, wherein the PRMT5 inhibitor is in amounts effective to inhibit proliferation of cells of the tumor.

31. A method of claim 1, which comprises:
administering to the subject an effective amount of a compound, or a pharmaceutically acceptable salt or product thereof or a stereoisomer thereof according to Formula I; 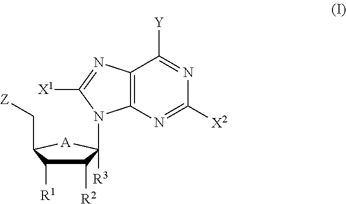
wherein:
A is —O—, —S—, or —NH—;
R¹ is H, OH, or L*;
R² and R³ are independently H, —OH, —Cl, —Br, —F, —I, —C₁-C₆ alkyl (e.g., methyl, ethyl, propyl), —(C₁-C₆)alkoxy (e.g., methoxy, ethoxy, propoxy), —(C₁-C₆)haloalkyl (e.g., CH₂F, CHF₂, CF₃), —(C₁-C₆)haloalkoxy, —CN, —NO₂, —NC, —NH₂, —N₃, or -L*;
X¹ and X² are independently —H, —Cl, —F or -L*;
Y is —NH₂, —OH, or -L*;
Z is -L*, —H, —OH, optionally substituted alkyl, -Q, or -D-Q,
wherein Q is phenyl or naphthyl wherein said phenyl and said naphthyl are optionally substituted once or twice by alkyl (e.g., methyl, ethyl, propyl), alkoxy (e.g., methoxy, ethoxy, propoxy), halo (e.g., —F, —Cl), haloalkyl (e.g., —CH₂F, —CHF₂, —CF₃), haloalkoxy (e.g., —OCH₂F, —OCHF₂, —OCF₃), cyano, nitro, nitrile; and,
wherein the optional substituents on the alkyl group are OH, halo (e.g., —F, —Cl), haloalkoxy (e.g., —OCH₂F, —OCHF₂, —OCF₃), alkoxy (e.g., methoxy, ethoxy, propoxy), cyano, nitro, nitrile;
D is —S—, —O— and —(CH₂)ₓ—
x is 1 to 6;
L* is a linker,
wherein for any given compound according to Formula I there can only be at most one L*.

32. A method of claim 1, which further comprises administering to the subject an effective amount of a compound, or a pharmaceutically acceptable salt or product selected from the group consisting of a CDK inhibitor and a HSP90 inhibitor.


SEQUENCE LISTING (36 sequences)

SEQ ID NO: 1 - DNA, 21 bp, Artificial Sequence
  source: /note=“Description of Artificial Sequence Synthetic oligonucleotide”
gcccagtttgagatgccttat

SEQ ID NO: 2 - DNA, 21 bp, Artificial Sequence
  source: /note=“Description of Artificial Sequence Synthetic oligonucleotide”
gcaaagtgaagtctttgtctt

SEQ ID NO: 3 - DNA, 137 bp, Artificial Sequence
  source: /note=“Description of Artificial Sequence Synthetic polynucleotide”
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 4 - DNA, 123 bp, Artificial Sequence
  source: /note=“Description of Artificial Sequence Synthetic polynucleotide”
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 5 - DNA, 110 bp, Artificial Sequence
  source: /note=“Description of Artificial Sequence Synthetic polynucleotide”
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 6 - DNA, 102 bp, Artificial Sequence
  source: /note=“Description of Artificial Sequence Synthetic polynucleotide”
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 7 - DNA, 88 bp, Artificial Sequence
  source: /note=“Description of Artificial Sequence Synthetic oligonucleotide”
  modified_base (1)..(20): a, c, t, g, unknown or other
nnnnnnnnnnnnnnnnnnnngttttagagctagaaatagcaagttaaaataaggctagtccgttatcaacttgaaaaagtgttttttt

SEQ ID NO: 8 - DNA, 76 bp, Artificial Sequence
  source: /note=“Description of Artificial Sequence Synthetic oligonucleotide”
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 9 - DNA, 20 bp, Homo sapiens
gagtccgagcagaagaagaa

SEQ ID NO: 10 - DNA, 20 bp, Homo sapiens
gagtcctagcaggagaagaa

SEQ ID NO: 11 - DNA, 20 bp, Homo sapiens
gagtctaagcagaagaagaa

SEQ ID NO: 12 - protein, 7 aa, Simian virus 40
PKKKRKV

SEQ ID NO: 13 - protein, 16 aa, Unknown
  source: /note=“Description of Unknown Nucleoplasmin bipartite NLS sequence”
KRPAATKKAGQAKKKK

SEQ ID NO: 14 - protein, 9 aa, Unknown
  source: /note=“Description of Unknown C-myc NLS sequence”
PAAKRVKLD

SEQ ID NO: 15 - protein, 11 aa, Unknown
  source: /note=“Description of Unknown C-myc NLS sequence”
RQRRNELKRSP

SEQ ID NO: 16 - protein, 38 aa, Homo sapiens
NQSSNFGPMKGGNFGGRSSGPYGGGGQYFAKPRNQGGY

SEQ ID NO: 17 - protein, 42 aa, Unknown
  source: /note=“Description of Unknown IBB domain from importin-alpha sequence”
RMRIZFKNKGKDTAELRRRRVEVSVELRKAKKDEQILKRRNV

SEQ ID NO: 18 - protein, 8 aa, Unknown
  source: /note=“Description of Unknown Myoma T protein sequence”
VSRKRPRP

SEQ ID NO: 19 - protein, 8 aa, Unknown
  source: /note=“Description of Unknown Myoma T protein sequence”
PPKKARED

SEQ ID NO: 20 - protein, 8 aa, Homo sapiens
PQPKKKPL

SEQ ID NO: 21 - protein, 12 aa, Mus musculus
SALIKKKKKMAP

SEQ ID NO: 22 - protein, 5 aa, Influenza virus
DRLRR

SEQ ID NO: 23 - protein, 7 aa, Influenza virus
PKQKKRK

SEQ ID NO: 24 - protein, 10 aa, Hepatitis delta virus
RKLKKKIKKL

SEQ ID NO: 25 - protein, 10 aa, Mus musculus
REKKKFLKRR

SEQ ID NO: 26 - protein, 20 aa, Homo sapiens
KRKGDEVDGVDEVAKKKSKK

SEQ ID NO: 27 - protein, 17 aa, Homo sapiens
RKCLQAGMNLEARKTKK

SEQ ID NO: 28 - DNA, 20 bp, Artificial Sequence
  source: /note=“Description of Artificial Sequence Synthetic oligonucleotide”
tctgcccgggagctaaaacg

SEQ ID NO: 29 - DNA, 20 bp, Artificial Sequence
  source: /note=“Description of Artificial Sequence Synthetic oligonucleotide”
gcagtcataatctgtcgcca

SEQ ID NO: 30 - DNA, 21 bp, Artificial Sequence
  source: /note=“Description of Artificial Sequence Synthetic oligonucleotide”
cctcaagaactccctggaata

SEQ ID NO: 31 - DNA, 21 bp, Artificial Sequence
  source: /note=“Description of Artificial Sequence Synthetic oligonucleotide”
gcgtttcaagagggagttcat

SEQ ID NO: 32 - DNA, 21 bp, Artificial Sequence
  source: /note=“Description of Artificial Sequence Synthetic oligonucleotide”
caagcctttctgagttgttta

SEQ ID NO: 33 - DNA, 21 bp, Artificial Sequence
  source: /note=“Description of Artificial Sequence Synthetic oligonucleotide”
tcgtattacaacgtcgtgact

SEQ ID NO: 34 - DNA, 21 bp, Artificial Sequence
  source: /note=“Description of Artificial Sequence Synthetic oligonucleotide”
acagttaaccactttttgaat

SEQ ID NO: 35 - DNA, 21 bp, Artificial Sequence
  source: /note=“Description of Artificial Sequence Synthetic oligonucleotide”
cctggaaggaagatgccttat

SEQ ID NO: 36 - DNA, 21 bp, Artificial Sequence
  source: /note=“Description of Artificial Sequence Synthetic oligonucleotide”
gcgtcttcatagatgcctcat